(19) United States
(12) Patent Application Publication
(10) Pub. No.: US 2017/0216125 A1
(43) Pub. Date: Aug. 3, 2017

(54) Walking Aid and Monitor

(71) Applicant: Elwha LLC, Bellevue, WA (US)

(72) Inventors: Roderick A. Hyde, Redmond, WA (US); Jordin T. Kare, San Jose, CA (US); William David Duncan, Sammamish, WA (US)

(73) Assignee: Elwha LLC

(21) Appl. No.: 15/433,595

(22) Filed: Feb. 15, 2017

Related U.S. Application Data
(63) Continuation-in-part of application No. 15/249,971, filed on Aug. 29, 2016, now Pat. No. 9,591,902.
Application No. 15/249,971 is a continuation of application No. 14/835,908, filed on Aug. 26, 2015, now Pat. No. 9,468,272.
Application No. 14/835,908 is a continuation-in-part of application No. 14/684,853, filed on Apr. 13, 2015.

(51) Int. Cl. A61H 3/00; A61B 5/0404; A45B 9/04; A61B 5/024; A61B 5/11; A61B 5/00; A61B 5/1455; A61B 5/01

(52) U.S. Cl. CPC A61H 3/00; A61B 5/14551; A61B 5/0404; A61B 5/01; A61B 5/024; A61B 5/112; A61B 5/6887; A45B 9/04; A61H 2201/5084; A61H 2201/501; A61H 2201/5048; A61H 2201/5092; A61H 2230/208; A61H 2230/045; A61H 2230/505; A61H 2230/065; A61H 2201/5043

(57) ABSTRACT

Devices and systems are described including a walking aid including a linear rod, a hand grip disposed in proximity to a first end of the linear rod, two or more extendable and retractable load-bearing feet at a second end of the linear rod, at least one first sensor configured to detect a parameter of a walking surface, at least one second sensor configured to detect a parameter of the walking aid, a controller including input circuitry configured to receive information regarding the detected parameter of the walking surface from the at least one first sensor and actuation circuitry configured to actuate at least one of the two or more extendable and retractable load-bearing feet in response to the information regarding the detected parameter of the walking surface, and a transmission unit including circuitry configured to transmit the second sensor output to a computing device.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

The present application constitutes a continuation-in-part of U.S. patent application Ser. No. 14/684,853, entitled STRUCTURAL FRAME TO ASSIST PATIENTS AND METHODS OF USE THEREOF, naming Mahalaxmi Gita Bangera, Jesse R. Cheatham, III, Hon Wah Chin, Roderick A. Hyde, Muriel Y. Ishikawa, Jordin T. Kare, Eric C. Leuthardt, Robert W. Lord, Richard T. Lord, Robert C. Petroski, Clarence T. Tegreene, Lowell L. Wood, Jr. as inventors, filed 13 Apr. 2015 with attorney docket no. 0313-006-001-000000, which is currently co-pending or is an application of which a currently co-pending application is entitled to the benefit of the filing date.
The present application constitutes a continuation-in-part of U.S. patent application Ser. No. 14/835,908, entitled SMART CANE WITH EXTENSIONS FOR NAVIGATING STAIRS, naming Roderick A. Hyde, Jordin T. Kare, William David Duncan as inventors, filed 26 Aug. 2015 with attorney docket no. 1213-002-017-000000, which is currently co-pending or is an application of which a currently co-pending application is entitled to the benefit of the filing date.
The present application constitutes a continuation-in-part of U.S. patent application Ser. No. 15/249,971 entitled SMART CANE WITH EXTENSIONS FOR NAVIGATING STAIRS, naming Roderick A. Hyde, Jordin T. Kare, William David Duncan as inventors, filed 29 Aug. 2016 with attorney docket no. 1213-002-017-000001, which is currently co-pending or is an application of which a currently co-pending application is entitled to the benefit of the filing date.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In an aspect, a walking aid includes, but is not limited to, a linear rod having a first end and a second end; a hand grip disposed in proximity to the first end of the linear rod; two or more extendable and retractable load-bearing feet at the second end of the linear rod, at least one of the two or more extendable and retractable load-bearing feet including two or more telescoping segments; at least one first sensor including circuitry configured to detect a parameter of a walking surface in proximity to the second end of the linear rod and to transform the detected parameter of the walking surface into a first sensor output; a controller including a microprocessor and circuitry, the controller operably coupled to the two or more extendable and retractable load-bearing feet and the at least one first sensor and including input circuitry configured to receive the first sensor output including information regarding the detected parameter of the walking surface; and actuation circuitry configured to actuate at least one of the two or more extendable and retractable load-bearing feet in response to the received information regarding the detected parameter of the walking surface; at least one second sensor including circuitry configured to detect a parameter of the walking aid and to transform the detected parameter of the walking aid into a second sensor output; and a transmission unit operably coupled to the at least one second sensor, the transmission unit including an antenna and circuitry configured to transmit the second sensor output to a computing device. In addition to the foregoing, other walking aid aspects are described in the claims, drawings, and text forming a part of the present disclosure.

In an aspect, a system includes, but is not limited to, a walking aid including a linear rod having a first end and a second end; a hand grip disposed in proximity to the first end of the linear rod; two or more extendable and retractable load-bearing feet at the second end of the linear rod, at least one of the two or more extendable and retractable load-bearing feet including two or more telescoping segments; at least one first sensor including circuitry configured to detect a parameter of a walking surface in proximity to the second end of the linear rod and to transform the detected parameter of the walking surface into a first sensor output; a controller including a microprocessor and circuitry, the controller operably coupled to the two or more extendable and retractable load-bearing feet and the at least one first sensor and including input circuitry configured to receive the first sensor output including information regarding the detected parameter of the walking surface, and actuation circuitry configured to actuate at least one of the two or more extendable and retractable load-bearing feet in response to the received information regarding the detected parameter of the walking surface; at least one second sensor including circuitry configured to detect a parameter of the walking aid and to transform the detected parameter of the walking aid into a second sensor output; and a transmission unit operably coupled to the at least one second sensor, the transmission unit including an antenna and circuitry configured to transmit the second sensor output to a computing device; and the computing device including a processor and circuitry including input circuitry configured to receive the second sensor output including information regarding the detected parameter of the walking aid; and calculation circuitry configured to calculate a gait value based on the received information regarding the detected parameter of the walking aid. In addition to the foregoing, other system aspects are described in the claims, drawings, and text forming a part of the present disclosure.

In an aspect, a walking aid includes, but is not limited to, a linear rod having a first end and a second end; a hand grip disposed in proximity to the first end of the linear rod; two or more extendable and retractable load-bearing feet at the second end of the linear rod, at least one of the two or more extendable and retractable load-bearing feet including two or more telescoping segments; at least one first sensor including circuitry configured to detect a parameter of a walking surface in proximity to the second end of the linear rod and to transform the detected parameter of the walking surface into a first sensor output; at least one second sensor including circuitry configured to detect a parameter of the walking aid and to transform the detected parameter of the walking aid into a second sensor output; a reporting device; and a computing component including a microprocessor and circuitry, the computing component operably coupled to the two or more extendable and retractable load-bearing feet, the at least one first sensor, the at least one second sensor, and the reporting device, and including input circuitry configured to receive the first sensor output including information regarding the detected parameter of the walking surface and to receive the second sensor output including information regarding the detected parameter of the walking aide; actuation circuitry configured to actuate at least one of the two or more extendable and retractable load-bearing feet in response to the received information regarding the detected parameter of the walking surface; calculation circuitry configured to calculate a gait value for a user based on the received information regarding the detected parameter of the walking aide; and reporting circuitry configured to transmit a signal to the reporting device in response to the calculated gait value. In addition to the foregoing, other walking aid aspects are described in the claims, drawings, and text forming a part of the present disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1A is a schematic of a walking aid and a user on a flat walking surface.

FIG. 1B is a schematic of a walking aid and a user on a stepped walking surface.

FIG. 2A is a schematic of a walking aid on a flat walking surface.

FIG. 2B is a schematic of a walking aid on a stepped walking surface.

FIG. 3A is a schematic of an extendable and retractable load-bearing foot with two or more telescoping segments in a retracted state.

FIG. 3B is a schematic of an extendable and retractable load-bearing foot with two or more telescoping segments in an extended state.

FIG. 3C is a schematic of an extendable and retractable load-bearing foot with a pivot connection and two or more telescoping segments in a retracted state.

FIG. 3D is a schematic of an extendable and retractable load-bearing foot with a pivot connection and two or more telescoping segments in an extended state.

FIG. 3E is a schematic of a walking aid with extendable and retractable load-bearing feet with a pivot connection and two or more telescoping segments on a flat walking surface.

FIG. 3F is a schematic of a walking aid with extendable and retractable load-bearing feet with a pivot connection and two or more telescoping segments on an uneven walking surface.

FIG. 3G is a schematic of a walking aid with extendable and retractable load-bearing feet with a pivot connection and two or more telescoping segments on a stepped walking surface.

FIG. 4A is a schematic of an extendable and retractable load-bearing foot with a pivot.

FIG. 4B is a schematic of an extendable and retractable load-bearing foot with two pivots.

FIG. 4C is a schematic of an extendable and retractable load-bearing foot with three pivots.

FIG. 4D is a schematic of an extendable and retractable load-bearing foot with a pivoting foot pad.

FIG. 4E is a schematic of a walking aid with extendable and retractable load-bearing feet with pivots on a flat walking surface.

FIG. 4F is a schematic of a walking aid with extendable and retractable load-bearing feet with pivots on an uneven walking surface.

FIG. 4G is a schematic of a walking aid with extendable and retractable load-bearing feet with pivots on a stepped walking surface.

FIG. 5A is a schematic of a walking aid with extendable and retractable load-bearing feet retracted for storage within an interior portion of the linear rod.

FIG. 5B is a schematic of a walking aid with extendable and retractable load-bearing feet pushed out of an interior portion of the linear rod.

FIG. 5C is a schematic of a walking aid with extendable and retractable load-bearing feet extended for use.

FIG. 6A is a schematic of a walking aid with extendable and retractable load-bearing feet retracted for storage along a side of the linear rod.

FIG. 6B is a schematic of a walking aid with extendable and retractable load-bearing feet extended for use.

FIG. 7 shows aspects of a walking aid such as depicted in FIG. 2A.

FIG. 8 illustrates aspects of a walking aid such as shown in FIG. 2A.

FIG. 9 depicts aspects of a walking aid such as illustrated in FIG. 2A.

FIG. 10 is a schematic of a system including a walking aid and a computing device.

FIG. 11 illustrates aspects of a system such as shown in FIG. 10.

FIG. 12 shows an embodiment of a system including a walking aid, a computing device, and remote signaling device.

FIG. 13 is a schematic of a walking aid.

FIG. 14 shows aspects of a walking aid such as shown in FIG. 13.

FIG. 15 illustrates aspects of a walking aid such as depicted in FIG. 13.

FIG. 16 depicts aspects of a walking aid such as illustrated in FIG. 13.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Described herein are walking aids configured to help a user walk on a walking surface as well as monitor the walking characteristics of the user. More specifically, described herein are walking aids including extendable and retractable load-bearing feet for stabilizing a walking aid and a user while walking on an uneven walking surface and sensors to monitor how the walking aid is used by the user to determine gait information. In some embodiments, the walking aid further includes physiological sensors for monitoring a physiological parameter of the user.

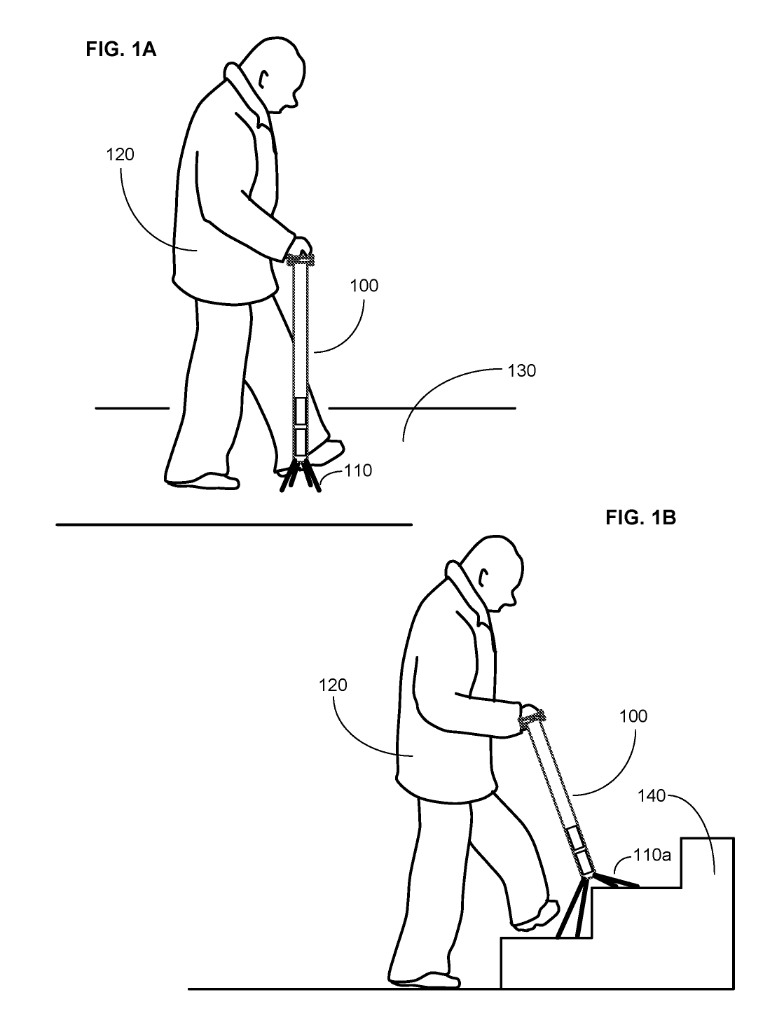

With reference to FIGS. 1A and 1B, shown is an example of a walking aid which can serve as a context for one or more devices, systems, and/or methods described herein. FIG. 1A shows a walking aid 100 including two or more extendable and retractable load-bearing feet 110. Walking aid 100 is shown in use by user 120 on a flat walking surface 130. FIG. 1B shows user 120 using walking aid 100 on a stepped walking surface 140. In this non-limiting example, the two or more extendable retractable load-bearing feet 110a are individually extended or retracted as needed to aid and stabilize the user 120 while climbing the stepped walking surface 140.

A walking aid such as described herein is configured for use on a variety of walking surfaces. In an aspect, the walking surface includes a sidewalk, a street, a dedicated walking path, a cobble stone or brick path, a dirt path, a grassy path, or a rocky path. In an aspect, the walking surface is uneven. For example, the walking surface can include an asphalt street including “pot holes”. For example, the walking surface can include a concrete sidewalk or dedicated walking path with buckled, broken, or uneven segments of concrete. For example, the walking surface can include a hiking trail with inclines and declines, rocks or boulders, tree roots, or other obstacles creating an uneven walking surface. In an aspect, the walking surface includes a slope, e.g., an incline or a decline. In an aspect, the walking surface includes a walking surface in a home or other building. In an aspect, the walking surface in a home or other building includes a carpeted walking surface, a hardwood floor walking surface, a laminate or linoleum walking surface, a tiled walking surface, or a stepped walking surface (e.g., a staircase). In an aspect, the walking surface includes a ramp with an inclined or declined slope. In an aspect, the walking surface includes a moving walking surface, e.g., a moving sidewalk or an escalator.

Described herein are aspects of a walking aid including a linear rod having a first end and a second end; a hand grip disposed in proximity to the first end of the linear rod; two or more extendable and retractable load-bearing feet at the second end of the linear rod, at least one of the two or more extendable and retractable load-bearing feet including two or more telescoping segments; at least one first sensor including circuitry configured to detect a parameter of a walking surface in proximity to the second end of the linear rod and to transform the detected parameter of the walking surface into a first sensor output; a controller including a microprocessor and circuitry, the controller operably coupled to the two or more extendable and retractable load-bearing feet and the at least one first sensor and including input circuitry configured to receive the first sensor output including information regarding the detected parameter of the walking surface; and actuation circuitry configured to actuate at least one of the two or more extendable and retractable load-bearing feet in response to the received information regarding the detected parameter of the walking surface; at least one second sensor including circuitry configured to detect a parameter of the walking aid and to transform the detected parameter of the walking aid into a second sensor output; and a transmission unit operably coupled to the at least one second sensor, the transmission unit including an antenna and circuitry configured to transmit the second sensor output to a computing device.

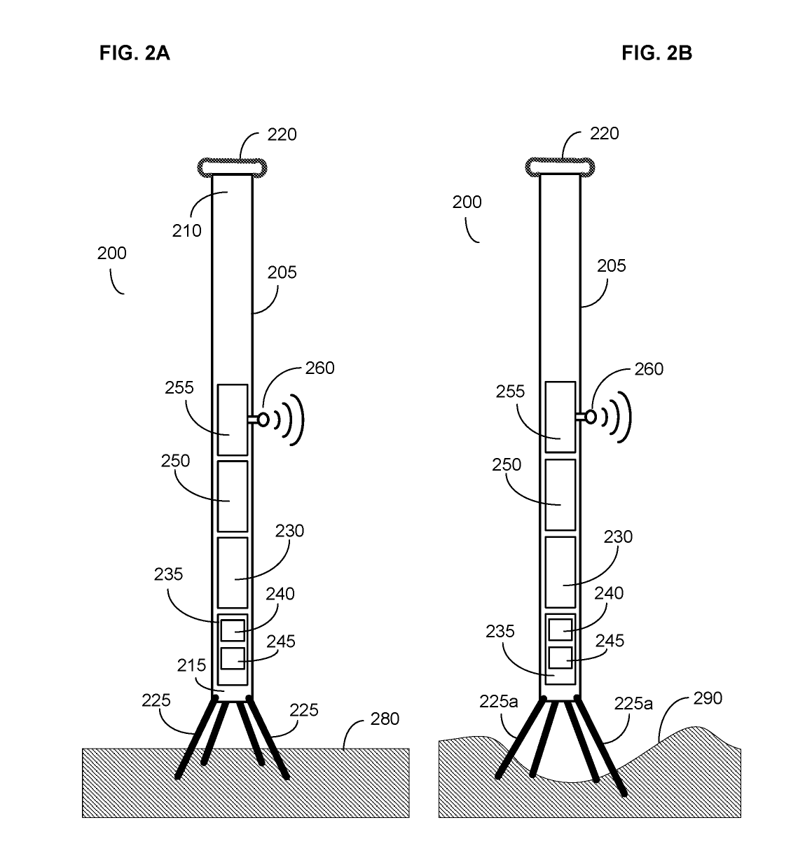

FIGS. 2A and 2B illustrate further aspects of a walking aid. FIG. 2A shows a schematic of walking aid 200 on walking surface 280, e.g., a relatively flat walking surface. Walking aid 200 includes a linear rod 205 having a first end 210 and a second end 215 and a hand grip 220 disposed in proximity to the first end 210 of the linear rod 205. Walking aid 200 further includes two or more extendable and retractable load-bearing feet 225 at the second end 215 of linear rod 205, at least one of the two or more extendable and retractable load-bearing feet 225 including two or more telescoping segments. Walking aid 200 includes at least one first sensor 230 including circuitry configured to detect a parameter of a walking surface 280 in proximity to the second end 215 of linear rod 205 and to transform the detected parameter of the walking surface 280 into a first sensor output. Walking aid 200 includes a controller 235 including a microprocessor 240 and circuitry 245. Controller 235 is operably coupled to the two or more extendable and retractable load-bearing feet 225 and the at least one first sensor 230. Circuitry 245 of controller 235 includes input circuitry configured to receive the first sensor output including information regarding the detected parameter of the walking surface 280 and actuation circuitry configured to actuate at least one of the two or more extendable and retractable load-bearing feet 225 in response to the information regarding the detected parameter of the walking surface 280. Walking aid 200 further includes at least one second sensor 250 including circuitry configured to detect a parameter of the walking aid 200 and to transform the detected parameter of the walking aid 200 into a second sensor output. Walking aid 200 further includes transmission unit 255 operably coupled to the at least one second sensor 250, the transmission unit 255 including an antenna 260 and circuitry configured to transmit the second sensor output to a computing device.

FIG. 2B shows a schematic of walking aid 200 on walking surface 290, e.g., an uneven walking surface. Walking aid 200 includes linear rod 205 and hand grip 220. Walking aid 200 further includes two or more extendable and retractable load-bearing feet 225a which have been individually extended or retracted to accommodate the uneven surface represented by walking surface 290. Walking aid 200 includes at least one first sensor 230 configured to detect a parameter, e.g., an unevenness, of walking surface 290 and controller 235 includes input circuitry configured to receive information regarding the detected parameter of walking surface 290 from the at least one first sensor 230 and actuation circuitry configured to actuate at least one of the two or more extendable and retractable load-bearing feet 225a in response to the information regarding the detected parameter of walking surface 290. The at least one second sensor 250 includes circuitry configured to detect a parameter of the walking aid 200 and to transform the detected parameter of the walking aid 200 into a second sensor output and the transmission unit 255 including an antenna 260 includes circuitry configured to transmit the second sensor output to a computing device.

Walking aid 200 such as described herein includes a linear rod 205. The linear rod provides the main structural support or shaft for the walking aid. The first end of the linear rod serves as a connection point for a hand grip. The second end of the linear rod serves as a connection point for the two or more extendable and retractable load-bearing feet. In an aspect, the linear rod provides an attachment point for the at least one first sensor, the at least one second sensor, the controller, and the transmission unit. In some embodiments, the linear rod provides an attachment point for one or more additional components of the walking aid. For example, the linear rod can provide an attachment point for a user-activated control mechanism, a user interface, at least one third sensor, an actuator, a mechanical energy harvester, a power source, and/or a warning system.

In an aspect, the linear rod comprises a cylindrical rod that is circular in transverse cross-section. Alternatively, the linear rod may have a non-circular transverse cross-section. For example, the linear rod can be oval, square, triangular, or polygonal in transverse cross-section. In an aspect, the diameter or width of the linear rod is about 0.5 inches to about 3.0 inches. For example, the diameter or width of the linear rod can be 0.5 inches, 0.6 inches, 0.7 inches, 0.8 inches, 0.9 inches, 1.0 inches, 1.1 inches, 1.2 inches, 1.3 inches, 1.4 inches, 1.5 inches, 1.6 inches, 1.7 inches, 1.8 inches, 1.9 inches, 2.0 inches, 2.1 inches, 2.2 inches, 2.3 inches, 2.4 inches, 2.5 inches, 2.6 inches, 2.7 inches, 2.8 inches, 2.9 inches, or 3.0 inches. A linear rod with a greater or lesser diameter or width is contemplated and may depend upon the composition and/or configuration of the linear rod.

In an aspect, the linear rod is about 12 inches to about 50 inches in length. For example, the linear rod can be 12 inches, 12.5 inches, 13 inches, 13.5 inches, 14 inches, 14.5 inches, 15 inches, 15.5 inches, 16 inches, 16.5 inches, 17 inches, 17.5 inches, 18 inches, 18.5 inches, 19 inches, 19.5 inches, 20 inches, 20.5 inches, 21 inches, 21.5 inches, 22 inches, 22.5 inches, 23 inches, 23.5 inches, 24 inches, 24.5 inches, 25 inches, 25.5 inches, 26 inches, 26.5 inches, 27 inches, 27.5 inches, 28 inches, 28.5 inches, 29 inches, 29.5 inches, 30 inches, 30.5 inches, 31 inches, 31.5 inches, 32 inches, 32.5 inches, 33 inches, 33.5 inches, 34 inches, 34.5 inches, 35 inches, 35.5 inches, 36 inches, 36.5 inches, 37 inches, 37.5 inches, 38 inches, 38.5 inches, 39 inches, 39.5 inches, 40 inches, 40.5 inches, 41 inches, 41.5 inches, 42 inches, 42.5 inches, 43 inches, 43.5 inches, 44 inches, 44.5 inches, 45 inches, 45.5 inches, 46 inches, 46.5 inches, 47 inches, 47.5 inches, 48 inches, 48.5 inches, 49 inches, 49.5 inches, or 50 inches in length. The length of the linear rod can be longer or shorter depending upon the overall desired length of the walking aid, e.g., the combined length of the hand grip, the linear rod, and the two or more extendable and retractable load-bearing feet. In an aspect, the length of the linear rod is adjustable. For example, the linear rod can include two or more telescoping segments with twistable locks to adjust and fix the length of the linear rod.

In an aspect, the linear rod is a solid linear rod. In an aspect, the hand grip, the two or more extendable and retractable load-bearing feet, the at least one first sensor, the at least one second sensor, the controller, and the transmission unit are attached to an outer surface of a solid linear rod. In an aspect, at least a portion of the linear rod is hollow. For example, at least a portion of the linear rod forms a hollow tube. In an aspect, at least a portion of the two or more extendable and retractable load-bearing feet, the at least one first sensor, the at least one second sensor, the controller, and the transmission unit are disposed within an interior portion of the linear rod.

Preferably the linear rod is formed from a lightweight but sturdy, weight-bearing material, non-limiting examples of which include metal or metal alloys (e.g., aluminum or an aluminum alloy; stainless steel; or titanium or a titanium alloy); a plastic or polymer material; a carbon fiber or a carbon fiber composite (e.g., carbon fiber-reinforced polymer or plastic; or carbon fiber-epoxy resin composite or a carbon fiber-polyester, -vinyl ester, or -nylon composite); a composite material (e.g., aramid, aluminum, ultra-high-molecular-weight polyethylene, or fiber glass); and/or wood (e.g., ash or beech wood).

Walking aid 200 such as described herein includes hand grip 220 disposed in proximity to the first end 210 of the linear rod 205. The hand grip is configured for gripping by a hand or hands of a user. In an aspect, the hand grip is disposed at the extreme end of the linear rod (e.g., attached to the top of the linear rod). In an aspect, the hand grip is incorporated into the first end of the linear rod (e.g., a portion of the first end of the linear rod at least partially covered by a rubberized material). In an aspect, the hand grip is a curved continuation of the linear rod (e.g., a curved portion at the first end of the linear rod that forms a hand grip). In an aspect, the hand grip is disposed in a region proximal to the first end of the linear rod (e.g., attached to a side of the linear rod in a region proximal to the first end of the linear rod, extended out from the linear rod like a handle). In an aspect, the hand grip is smooth. In an aspect, the hand grip is contoured to accommodate the fingers of a gripping hand (e.g., having an ergonomically compatible grip or a contoured gel grip). In an aspect, the hand grip is formed from the same material used to form the linear rod. In an aspect, the hand grip is a contiguous part of the linear rod. In an aspect, the hand grip is formed separately and subsequently attached to the linear rod. In an aspect, the hand grip is formed from a different material from that used to form the linear rod. In an aspect, the hand grip is formed from a metal or metal alloy (e.g., aluminum, stainless steel, titanium, brass, or silver); plastic (e.g., polyvinylchloride); foam (e.g., polyurethane foam rubber or latex foam rubber); rubber; wood; or cork.

In an aspect, walking aid 200 includes two or more extendable and retractable load-bearing feet 225. In an aspect, a walking aid includes two to ten extendable and retractable load-bearing feet. For example, a walking aid can include two, three, four, five, six, seven, eight, nine, or ten extendable and retractable load-bearing feet. In an aspect, each of the two or more extendable and retractable load-bearing feet is about 2 inches to about 24 inches in length. For example, at least one of the two or more extendable and retractable load-bearing feet is 2 inches, 2.5 inches, 3 inches, 3.5 inches, 4 inches, 4.5 inches, 5 inches, 5.5 inches, 6 inches, 6.5 inches, 7 inches, 7.5 inches, 8 inches, 8.5 inches, 9 inches, 9.5 inches, 10 inches, 10.5 inches, 11 inches, 11.5 inches, 12 inches, 12.5 inches, 13 inches, 13.5 inches, 14 inches, 14.5 inches, 15 inches, 15.5 inches, 16 inches, 16.5 inches, 17 inches, 17.5 inches, 18 inches, 18.5 inches, 19 inches, 19.5 inches, 20 inches, 20.5 inches, 21 inches, 21.5 inches, 22 inches, 22.5 inches, 23 inches, 23.5 inches, or 24 inches in length. The length of each of the extendable and retractable load-bearing feet can be longer or shorter depending upon the overall desired length of the walking aid, e.g., the combined length of the hand grip, the linear rod, and the two or more extendable and retractable load-bearing feet.

In an aspect, at least one of the two or more extendable and retractable load-bearing feet is circular in transverse cross-section. Alternatively, at least one of the two or more extendable and retractable load-bearing feet may include a non-circular transverse cross-section. For example, at least one of the two or more extendable and retractable load-bearing feet can be oval, square, triangular, or polygonal in transverse cross-section. In an aspect, the diameter or width of at least one of the two or more extendable and retractable load-bearing feet is about 0.2 inches to about 3.0 inches. For example, the diameter or width of at least one of the two or more extendable and retractable load-bearing feet can be 0.2 inches, 0.3 inches, 0.4 inches, 0.5 inches, 0.6 inches, 0.7 inches, 0.8 inches, 0.9 inches, 1.0 inches, 1.1 inches, 1.2 inches, 1.3 inches, 1.4 inches, 1.5 inches, 1.6 inches, 1.7 inches, 1.8 inches, 1.9 inches, 2.0 inches, 2.1 inches, 2.2 inches, 2.3 inches, 2.4 inches, 2.5 inches, 2.6 inches, 2.7 inches, 2.8 inches, 2.9 inches, or 3.0 inches. An extendable and retractable load-bearing foot with a larger or smaller diameter or width is contemplated and may depend upon the composition and/or configuration of the extendable and retractable load-bearing foot.

Preferably, the two or more extendable and retractable load-bearing feet are formed from a lightweight but sturdy material, e.g., a material capable of load-bearing, non-limiting examples of which include metal or metal alloys (e.g., aluminum or an aluminum alloy; stainless steel; or titanium or a titanium alloy); a plastic or polymer material; a carbon fiber or a carbon fiber composite (e.g., carbon fiber-reinforced polymer or plastic; or carbon fiber-epoxy resin composite or a carbon fiber-polyester, -vinyl ester, or -nylon composite); a composite material (e.g., aramid, aluminum, ultra-high-molecular-weight polyethylene, or fiber glass); and/or wood (e.g., ash or beech wood).

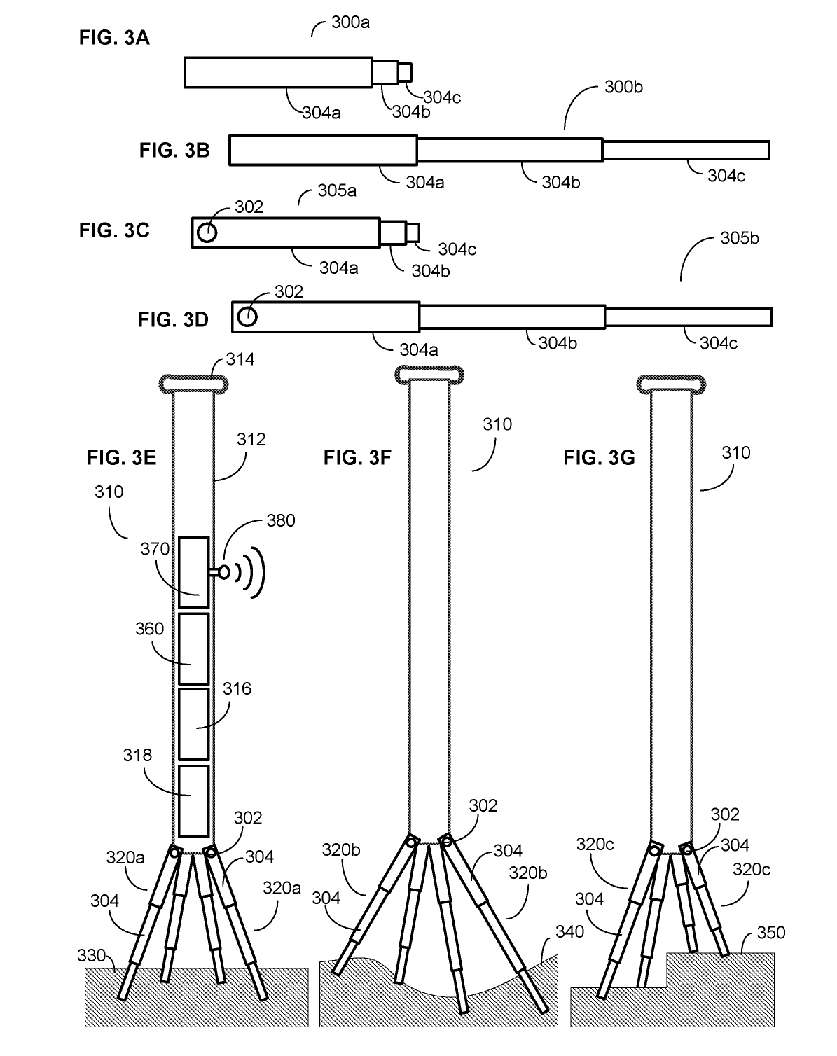

At least one of the two or more extendable and retractable load-bearing feet 225 of walking aid 200 includes two or more telescoping segments. FIGS. 3A and 3B illustrate aspects of extendable and retractable load-bearing feet with two or more telescoping segments. FIG. 3A shows an example of a single extendable and retractable load-bearing foot 300a in a retracted configuration. Extendable and retractable foot 300a includes telescoping segments 304a, 304b, and 304c. Telescoping segment 304c is shown at least partially disposed within telescoping segment 304b, and telescoping segment 304b is shown at least partially disposed within telescoping segment 304a. FIG. 3B shows extendable and retractable foot 300b in an extended configuration. In the extended configuration, telescoping segments 304a, 304b, and 304c have been extended relative to one another.

In an aspect, an extendable and retractable load-bearing foot includes two telescoping segments. For example, an extendable and retractable load-bearing foot can include a first segment configured to slide into or out of an interior portion of an adjacent second segment to adjust the overall length of the foot. In an aspect, an extendable and retractable load-bearing foot can include three or more telescoping segments. In an aspect, at least one of the telescoping segments is a hollow tube of a load-bearing metal or metal alloy. For example, at least one of the telescoping segments can be a hollow tube of aluminum, titanium, or alloys thereof. In an aspect, at least one of the telescoping segments is a hollow tube of load-bearing plastic or polymer. For example, at least one of the telescoping segments can be a hollow tube of carbon fiber-epoxy resin composite.

In an aspect, at least one of the two or more extendable and retractable load-bearing feet has a pivot at a connection to the linear rod. For example, the at least one of the two or more extendable and retractable load-bearing feet having the two or more telescoping segments can further include a pivot at a connection to the linear rod. In some embodiments, at least one first of the two or more extendable and retractable load-bearing feet includes the two or more telescoping segments and at least one second of the two or more extendable and retractable load-bearing feet includes the pivot at a connection to the linear rod. In an aspect, at least one of the two or more extendable and retractable load-bearing feet is rotatable. For example at least one of the two or more extendable and retractable feet can be rotatable around a pivot at a connection to the linear rod. FIGS. 3C and 3D illustrate aspects of extendable and retractable load-bearing feet including a pivot at a connection to the linear rod and two or more telescoping segments. FIG. 3C shows a single extendable and retractable load-bearing foot 305a in a retracted configuration and pivot 302 for connecting the extendable and retractable load-bearing foot to a portion of a linear rod. Extendable and retractable load-bearing foot 305a is able to rotate relative to the linear rod around pivot 302. Extendable and retractable foot 305a includes telescoping segments 304a, 304b, and 304c. Telescoping segment 304c is shown at least partially disposed within telescoping segment 304b, and telescoping segment 304b is shown at least partially disposed within telescoping segment 304a. FIG. 3D shows extendable and retractable foot 305b in an extended configuration. In the extended configuration, telescoping segments 304a, 304b, and 304c have been extended relative to one another.

FIGS. 3E-3G illustrate aspects of a walking aid including extendable and retractable load-bearing feet having a pivot at a connection to the linear rod and two or more telescoping segments. FIG. 3E shows walking aid 310 on walking surface 330. Walking aid 310 includes linear rod 312 having a first end and a second end, hand grip 314 disposed in proximity to the first end of linear rod 312, two or more extendable and retractable load-bearing feet 320a at the second end of linear rod 312, the two or more extendable and retractable load-bearing feet 320a having a pivot 302 at a connection to the linear rod 312 and two or more telescoping segments 304, at least one first sensor 316 configured to detect a parameter of walking surface 330 in proximity to the second end of linear rod 312, and a controller 318 including a microprocessor and circuitry and operably coupled to the two or more extendable and retractable load-bearing feet 320a and the at least one first sensor 316, controller 318 including input circuitry configured to receive the first sensor output including information regarding the detected parameter of walking surface 330 and actuation circuitry configured to actuate at least one of the two or more extendable and retractable load-bearing feet 320a in response to the information regarding the detected parameter of walking surface 330. Walking aid 310 further includes at least one second sensor 360 including circuitry configured to detect a parameter of the walking aid 310 and to transform the detected parameter of the walking aid into a second sensor output and a transmission unit 370 operably coupled to the at least one second sensor 360 and including an antenna 380 and circuitry configured to transmit the second sensor output to a computing device.

FIG. 3F shows walking aid 310 of FIG. 3E on walking surface 340. Walking surface 340 represents an uneven walking surface. In this non-limiting example, the two or more extendable and retractable load-bearing feet 320a of FIG. 3E have been extended or retracted by pivoting around pivot 302 and/or telescoping segments 304 represented by two or more extendable and retractable load-bearing feet 320b. The extension and retraction around pivot points 302 or telescoping of segments 304 is based on the information regarding a detected parameter, e.g., unevenness, of walking surface 340. FIG. 3G shows walking aid 310 of FIG. 3E on walking surface 350. Walking surface 350 represents a stepped walking surface. In this non-limiting example, the two or more extendable and retractable load-bearing feet 320a of FIG. 3E have been extended or retracted by pivoting around pivot 302 and/or telescoping segments 304 represented by two or more extendable and retractable load-bearing feet 320c. The extension and retraction around pivot points 302 or telescoping of segments 304 is based on the information regarding a detected parameter, e.g., stepped, of walking surface 350.

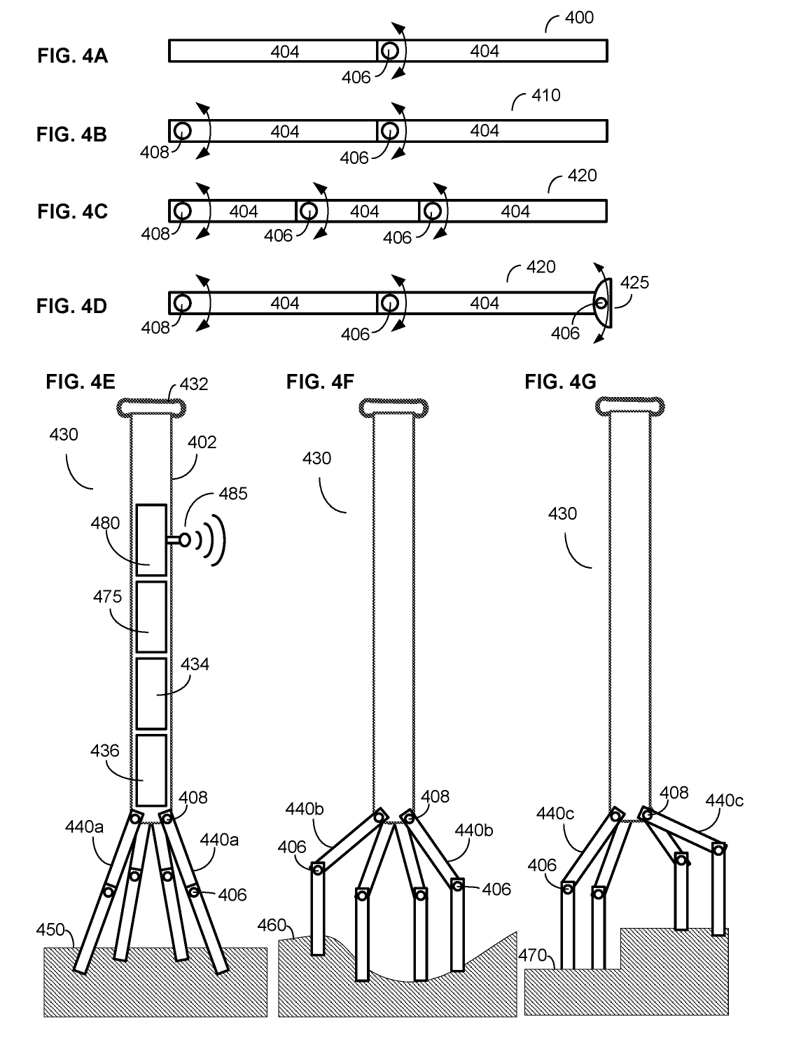

In some embodiments, at least one of the two or more extendable and retractable load-bearing feet has a pivot at a connection to the linear rod and one or more additional pivots along a length of the at least one of the two or more extendable and retractable load-bearing feet. FIGS. 4A-4C illustrate non-limiting examples of an extendable and retractable load-bearing foot with one or more pivot points. FIG. 4A shows extendable and retractable load-bearing foot 400 including two segments 404 connected by a pivot 406. In this non-limiting example, the extendable and retractable load-bearing foot has a non-pivoting connection to a portion of the linear rod and a single pivot point between the two segments. FIG. 4B shows extendable and retractable load-bearing foot 410 including two segments 404 connected by a pivot 406. An additional pivot 408 can be used to connect extendable and retractable load-bearing foot 410 to a portion of the linear rod. In this non-limiting example, the extendable and retractable load-bearing foot has two pivot points. FIG. 4C shows extendable and retractable load-bearing foot 420 including three segments 404 attached to one another in a series through pivots 406. An additional pivot 408 can be used to connect extendable and retractable load-bearing foot 400 to a portion of the linear rod. In this non-limiting example, the extendable and retractable load-bearing foot 420 includes three pivot points.

In some embodiments, at least one of the two or more extendable and retractable load-bearing feet includes an additional foot pad at the distal end of the at least one of the two or more extendable and retractable load-bearing feet. For example, the foot pad can include a rubber pad or cup attached to the distal end of the extendable and retractable load-bearing foot. For example, a rubber pad or cup can be added to prevent slippage of the extendable and retractable load-bearing feet on a slick walking surface. In an aspect, the foot pad includes a pivot connection to the extendable and retractable load-bearing foot. For example, the foot pad can include a pivot connection that allows it to pivot relative to movement of the extendable and retractable load-bearing foot to which it is attached. FIG. 4D shows extendable and retractable load-bearing foot 430 including foot pad 425. Extendable and retractable load-bearing foot 430 includes two segments 404 connected by pivot 406. Extendable and retractable load-bearing foot 430 is configured for attached to a portion of a linear rod through another pivot 408. Similarly, foot pad 425 is connected to segment 404 at the distal end of extendable and retractable load-bearing foot 430 through a third pivot 406.

FIGS. 4E-4G illustrate aspects of a walking aid including extendable and retractable load-bearing feet having a pivot at a connection to the linear rod and one or more additional pivots along the length of the at least one of the two or more extendable and retractable load-bearing feet. FIG. 4E shows walking aid 430 on walking surface 450. Walking aid 430 includes linear rod 402 having a first end and a second end, hand grip 432 disposed in proximity to the first end of linear rod 402, two or more extendable and retractable load-bearing feet 440a at the second end of linear rod 402, the two or more extendable and retractable load-bearing feet 440a having a pivot 408 at a connection to the linear rod 402 and an additional pivot 406 along the length of the two or more extendable and retractable load-bearing feet 440a, at least one first sensor 434 configured to detect a parameter of walking surface 450 in proximity to the second end of linear rod 402, and a controller 436 including a microprocessor and circuitry and operably coupled to the two or more extendable and retractable load-bearing feet 440a and the at least one first sensor 434, controller 436 including input circuitry configured to receive the first sensor output including information regarding the detected parameter of walking surface 450 and actuation circuitry configured to actuate at least one of the two or more extendable and retractable load-bearing feet 440a in response to the information regarding the detected parameter of walking surface 450. Walking aid 430 further includes at least one second sensor 475 including circuitry configured to detect a parameter of the walking aid 430 and to transform the detected parameter of the walking aid into a second sensor output and a transmission unit 480 operably coupled to the at least one second sensor 475 and including an antenna 485 and circuitry configured to transmit the second sensor output to a computing device.

FIG. 4F shows walking aid 430 of FIG. 4E on walking surface 460.

Walking surface 460 represents an uneven walking surface. In this non-limiting example, the two or more extendable and retractable load-bearing feet 440a of FIG. 4E have been extended or retracted around pivot points 406 and 408 represented by two or more extendable and retractable load-bearing feet 440b. The extension and retraction around pivot points 406 and 408 is based on the information regarding a detected parameter, e.g., unevenness, of walking surface 460. FIG. 4G shows walking aid 430 of FIG. 4E on walking surface 470. Walking surface 470 represents a stepped walking surface. In this non-limiting example, the two or more extendable and retractable load-bearing feet 440a of FIG. 4E have been extended or retracted around pivot points 406 and 408 represented by two or more extendable and retractable load-bearing feet 440c. The extension and retraction around pivot points 406 and 408 is based on the information regarding a detected parameter, e.g., stepped, of walking surface 470.

In some embodiments, the two or more extendable and retractable load-bearing feet are operable to make the walking aid free-standing. For example, the two or more extendable and retractable load-bearing feet are operable to be moved into a position relative to one another that provides a stable platform for holding the walking aid in a near vertical position. In an aspect, a walking aid includes two or more extendable and retractable load-bearing feet including two or more telescoping segments operable to make the walking aid free-standing. In an aspect, a walking aid includes two or more extendable and retractable load-bearing feet including a pivot connection to a linear rod and two or more telescoping segments operable to make the walking aid free-standing. In an aspect, a walking aid includes two or more extendable and retractable load-bearing feet including a pivot connection to a linear rod and one or more additional pivots along the length of the two or more extendable and retractable load-bearing feet and operably to make the walking aid free-standing.

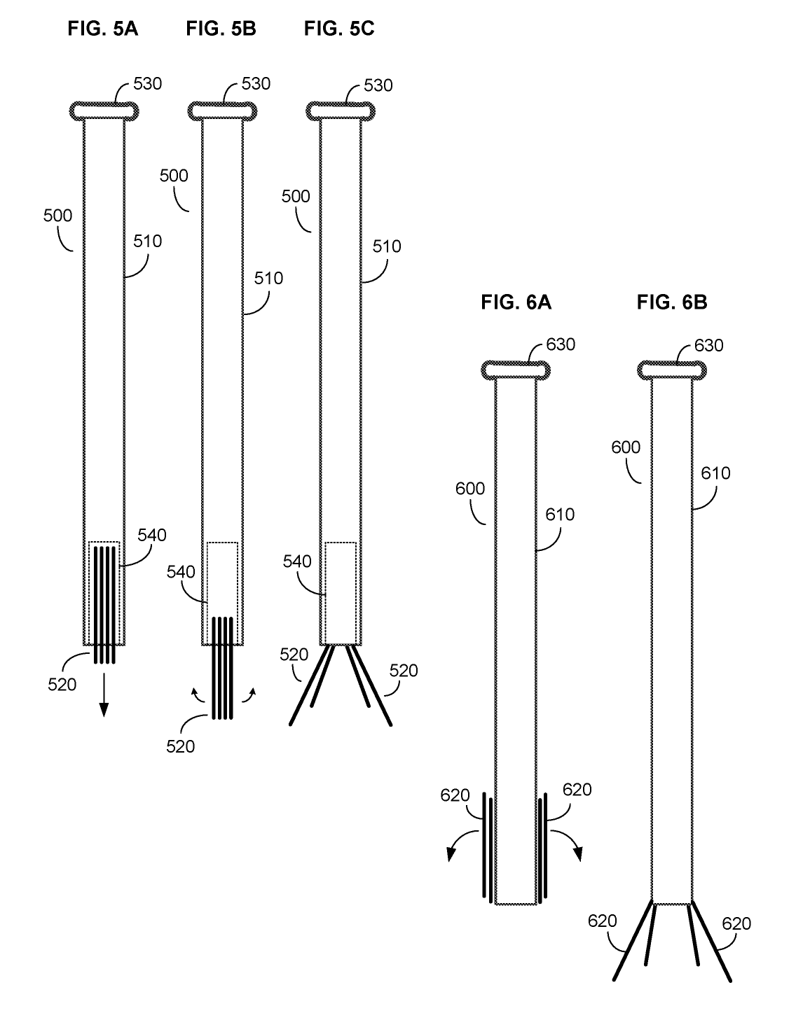

In some embodiments, at least one of the two or more extendable and retractable load-bearing feet is configured to retract for storage into an interior portion of the linear rod. FIGS. 5A-5C illustrate a non-limiting example of a walking aid including extendable and retractable load-bearing feet retracted for storage into an interior portion of the linear rod of the walking aid. In FIG. 5A, walking aid 500 includes linear rod 510, two or more extendable and retractable load-bearing feet 520, and hand grip 530. The two or more extendable and retractable load-bearing feet 520 are shown retracted for storage within an interior portion 540 (dashed outline) constituting a compartment or hollow space inside a portion of the linear rod. In FIG. 5B, shows walking aid 500 including hand grip 530 with the two or more extendable and retractable load-bearing feet 520 pushed out of the interior portion 540 of the linear rod 510. For example, the two or more extendable and retractable load-bearing feet can be attached at one end to a motor including pistons and/or pulleys that are capable of pushing and pulling the two or more extendable and retractable load-bearing feet out of and into the interior portion of the linear rod. In FIG. 5C, shows walking aid 500 with the two or more extendable and retractable load-bearing feet 520 fully extended from the interior portion 540 of the linear rod 510 and pivoted to provide support on a walking surface. In some embodiments, a user-activated control mechanism associated with hand grip 530, e.g., a button or switch, is used to initiate pushing or pulling of the two or more extendable and retractable load-bearing feet 520 into or out of the interior portion 540 of linear rod 510.

In some embodiments, at least one of the two or more extendable and retractable load-bearing feet is configured to retract for storage on a side of the linear rod. FIGS. 6A and 6B illustrate a non-limiting example of a walking aid including extendable and retractable load-bearing feet retracted for storage on a side of the walking aid. In FIG. 6A, walking aid 600 includes linear rod 610, two or more extendable and retractable load-bearing feet 620, and hand grip 630. The two or more extendable and retractable load-bearing feet 620 are shown retracted or folded up for storage along the outer surface of the linear rod 610. In FIG. 6B, walking aid 600 is shown with the two or more extendable and retractable load-bearing feet 620 pivoted or unfolded outward from the outer surface of the linear rod 610 and ready for contact with a walking surface. In some embodiments, a user-activated control mechanism associated with hand grip 630, e.g., a button, is used to initiate pivoting or unfolding of the two or more extendable and retractable load-bearing feet 620.

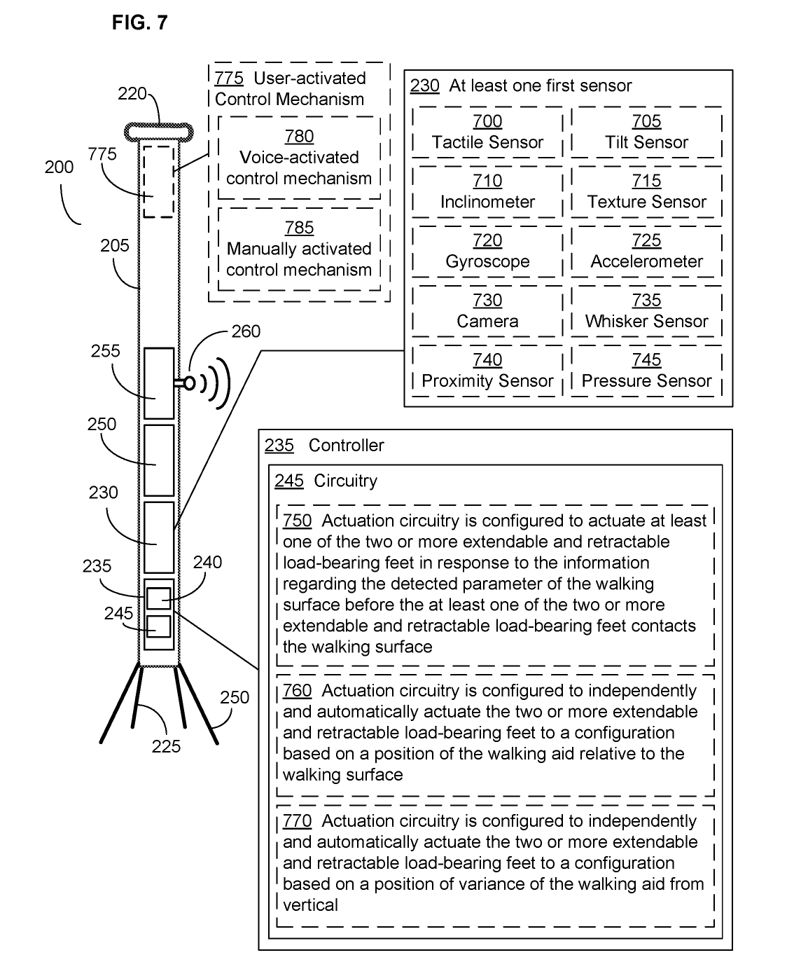

FIG. 7 illustrates further aspects of a walking aid such as shown in FIG. 2A. Walking aid 200 includes at least one first sensor 230 configured to detect a parameter of a walking surface in proximity to the second end of the linear rod 205. In an aspect, the parameter of the walking surface includes at least one of a surface type, a friction coefficient, a roughness, a slope, an edge, a height, a distance, or a bump.

In an aspect, the at least one first sensor 230 is incorporated into an interior portion of linear rod 205. For example, an accelerometer, tilt sensor, or inclinometer can be incorporated into an interior portion of the linear rod. In an aspect, the at least one first sensor 230 is incorporated onto an outer surface of linear rod 205. For example, a camera or proximity sensor can be attached to an outer surface of the linear rod. In an aspect, the at least one first sensor 230 is incorporated into hand grip 220. In some embodiments, the at least one first sensor 230 is associated with at least one of the two or more extendable and retractable load-bearing feet 225. For example, that portion of an extendable and retractable load-bearing foot that makes contact with the walking surface can include one or more sensors, e.g., one or more pressure sensors.

In an aspect, the at least one first sensor 230 comprises at least one of a tactile sensor 700, a tilt sensor 705, an inclinometer 710, or a texture sensor 715. In an aspect, the at least one first sensor 230 includes a tactile sensor 700 configured to detect a parameter of a walking surface in proximity to the second end of the linear rod. In an aspect, the tactile sensor is configured to assess the hardness (e.g., concrete or asphalt) or softness (deep sand or grass) of the walking surface. In an aspect, the tactile sensor 700 includes a pressure sensor. For example, the tactile sensor can include a pressure sensor array, for determining weight, texture, stiffness, center of mass, and/or coefficient of friction. In an aspect, the tactile sensor can include a force gauge or force sensor. In an aspect, the tactile sensor includes an array of strain gauges. In an aspect, the tactile sensor can include a piezoresistive, a piezoelectric, capacitive, an optical sensor, an ultrasonics-based sensor, a magnetism-based sensor, or elastoresistive tactile sensor. See, e.g., Dahiya et al. (2009) “Tactile Sensing—From Humans to Humanoids” IEEE Transactions on Robotics, 26:1-20, which is incorporated herein by reference. In an aspect, the tactile sensor is configured to detect slippage and friction coefficient. See, e.g., Chuang et al. (2012) “Detection system of incident slippage and friction coefficient based on a flexible tactile sensor with structural electrodes” Sensors and Actuators A 188:48-55; and Nakamura & Shinoda (2001) “A tactile sensor instantaneously evaluating friction coefficients” The 11^{th} International Conference on Sold-State Sensors and Actuators, Munich, Germany, Jun. 10-14, 2001, pp. 1430-1433, which are incorporated herein by reference.

In an aspect, the at least one first sensor 230 includes at least one of a tilt sensor 705 or an inclinometer 710 configured to detect a parameter of a walking surface in proximity to the second end of the linear rod. In an aspect, the tilt sensor or the inclinometer includes at least one of a tilt meter, a tilt indicator, a slope alert, a slope gauge, a gradient meter, a gradiometer, a level gauge, a declinometer, and a pitch and roll indicator. In an aspect, the inclinometer measures incline (positive slope as observed by user) or decline (negative slope as observed by user). For example, a tilt sensor or inclinometer can be used to indicate a slope, either positive or negative, in proximity to the walking aid. In some embodiments, the tilt sensor or inclinometer monitors changes in a parameter of the walking aid, e.g., tilt or inclination, relative to the walking surface. In an aspect, the tilt sensor or inclinometer measures pitch and roll of the walking aid. In an aspect, a tilt sensor or an inclinometer includes a three-axis accelerometer. Tilt sensors and inclinometers are available from commercial sources (from, e.g., Trossen Robotics, Downers Grove, Ill.; Dimension Engineering, Akron, Ohio).

In an aspect, the at least one first sensor 230 includes a texture sensor 715 configured to detect a parameter of a walking surface in proximity to the second end of the linear rod. For example, the texture sensor can be configured to detect a texture, e.g., rough or smooth, of the walking surface. In an aspect, the texture sensor is configured to detect light reflected off the surface of the walking surface. For example, a shiny surface should reflect light at an angle equal to the angle of incidence of the surface relative to a plane tangent to the point of incidence. In contrast, a rough or matte surface scatters incident light. In an aspect, the texture sensor includes a light source, e.g, a laser, and photoreceptors configured to detect the reflected light. In an aspect, the texture sensor is incorporated into a distal end of the extendable and retractable load-bearing feet and in physical contact with the walking surface. See, e.g., Ye et al. (2007) “Pen-type Sensor for Surface Texture Perception” 16^{th} IEEE International Conference on Robot & Human Interactive Communication, Aug. 26-29, 2007, Jeju, Korea, which is incorporated herein by reference. In an aspect, the texture sensor includes a tactile sensor array. See, e.g., Jamali et al. (2009) “Texture Recognition by Tactile Sensing” Australasian Conference on Robotics and Automation (ACRA), Dec. 2-4, 2009, Sydney, Australia, which is incorporated herein by reference.

In an aspect, the at least one first sensor 230 comprises at least one of an accelerometer 725 or a gyroscope 720. In an aspect, at least one of accelerometer 725 or gyroscope 720 are configured to detect a parameter of a walking surface in proximity to the second end of the linear rod. In an aspect, the accelerometer senses acceleration of the walking aid in one, two, or three axes. For example, the accelerometer can include a three-axis sensor for measuring accelerations on three accelerometer axes. The signal from an accelerometer sensor can be separated into two signals: the acceleration from gravity, and external acceleration. The acceleration from gravity allows measurement of the tilt of the sensor by identifying which direction is “down.” In an aspect, the accelerometer includes a proof mass-spring system. In an aspect, the accelerometer is a piezoelectric accelerometer, a piezoresistive accelerometer, or a capacitive accelerometer. In an aspect, the gyroscope can include a MEMS gyroscope, solid state ring laser gyroscope, fiber optic gyroscope, or a quantum gyroscope. Accelerometers and/or gyroscopes are available from commercial sources (from, e.g., Meggit Sensing Systems, Irvine, Calif.; Trossen Robotics, Downers Grove, Ill.; Dimension Engineering, Akron, Ohio; Freescale Semiconductor Inc., Austin, Tex.).

In an aspect, accelerometer 725 is configured to detect height differences between successive contacts of a portion of the walking aid with the walking surface. For example, one or more accelerometers can be used to indicate successive increases in the height of the walking aid between successive contacts with a stepped walking surface. In an aspect, accelerometer 725 is configured to be located on a user. For example, an accelerometer on the user can provide information regarding the progress of the user in a forward, backward, or lateral direction.

In an aspect, the at least one first sensor 230 comprises at least one camera 730. In an aspect, at least one camera 730 is configured to detect a parameter of a walking surface in proximity to the second end of the linear rod. For example, the at least one camera can include a digital camera configured to capture one or more images of the walking surface in proximity to the second end of the linear rod. In an aspect, the at least one camera includes a camera module including a CMOS or CCD image sensor. Digital camera modules are available from commercial sources (from, e.g., e-con Systems, Inc., St. Louis, Mo.; STMicroelectronics, Geneva, Switzerland).

In an aspect, the at least one first sensor 230 comprises at least one whisker sensor 735. In an aspect, the at least one whisker sensor 735 is configured to detect a parameter of the walking surface in proximity to the second end of the linear rod. For example, the whisker sensor can include one or more flexible wires attached to the second end of the linear rod of the walking aid, wherein movement of the one or more flexible wires in response to touching an object on the walking surface, e.g., an edge of an uneven walkway, is detected. In an aspect, the at least one whisker sensor is attached to one of the two or more extendable and retractable load-bearing feet. See, e.g., Wijaya & Russell (2002) “Object Exploration using Whisker Sensors” Proc. 2002 Australasian Conference on Robotics and Automation, Auckland, 27-29 Nov. 2002, pp. 180-185, which is incorporated herein by reference.

In an aspect, the at least one first sensor 230 comprises at least one proximity sensor 740. In an aspect, the at least one proximity sensor 740 is configured to detect a parameter of the walking surface in proximity to the second end of the linear rod. For example, the walking aid can include at least one proximity sensor to sense or detect how close a portion of the walking surface is to a portion of the walking aid. In an aspect, the at least one proximity sensor comprises at least one of a photoelectric proximity sensor, an acoustic proximity sensor, or a capacitive proximity sensor. In an aspect, the at least one proximity sensor includes at least one of an inductive proximity sensor, long range proximity sensor, ultrasonic proximity sensor, infrared (IR) proximity sensor, 3gs proximity sensor, RFID proximity sensor, laser proximity sensor, wireless proximity sensor, proximity sensor alarm, micro proximity sensor, or microwave proximity sensor. In an aspect, the proximity sensor includes at least one camera configured to capture images in proximity to the walking aid. Proximity sensors are available from a variety of commercial sources (from, e.g., ZF Electronics Corp., Pleasant Prairie, Wis.; Freescale Semiconductor Inc., Austin, Tex.; Semtech, Camarillo, Calif.; STMicroelectronics, Geneva, Switzerland). In an aspect, the at least one first sensor 230 comprises at least one pressure sensor 745. In an aspect, the at least one pressure sensor 745 is configured to detect a parameter of a walking surface in proximity to the second end of the linear rod. In an aspect, the at least one pressure sensor includes a capacitive pressure sensor. For example, the pressure sensor can include a two metal plates separated by a nonconductive foam to form a capacitive transducer that when connected to an inductor results in an inductance-capacitance combination with a specific frequency of oscillation. Applying pressure to the sensor alters the distance between the metal places, lowers the oscillator frequency, and increases the capacitance. In an aspect, the at least one pressure sensor includes an elastomer pressure sensor. For example, the pressure sensor can include a compressible foam pad with electrodes on one side receiving a positive voltage and mated pairs of electrodes on the other side receiving a negative voltage. Compression of the pad reduces the resistance between the electrode pairs and increase in current. Pressure sensors are commercially available from a variety of sources (from, e.g., Trossen Robotics, Downers Grove, Ill.; Freescale Semiconductor Inc., Austin, Tex.).

Returning to FIG. 7, walking aid 200 includes a controller 235 including a microprocessor 240 and circuitry 245. Controller 235 is operably coupled to the two or more extendable and retractable load-bearing feet 225 and the at least one first sensor 230 and includes circuitry 245 including input circuitry configured to receive information regarding the detected parameter of the walking surface from the at least one first sensor 230 and actuation circuitry configured to actuate at least one of the two or more extendable and retractable load-bearing feet 225 in response to the information regarding the detected parameter of the walking surface.

In an aspect, controller 235 includes a microprocessor 240. In an aspect, microprocessor 240 includes a central processing unit for controlling one or more functions of the walking aid. The controller further includes a system memory and a system bus that couples various system components including the system memory to the microprocessor. The microprocessor can include a processing unit, a central processing unit (CPU), a digital signal processor (DSP), an application-specific integrated circuit (ASIC), a field programmable gate entry (FPGA), or the like, or any combinations thereof, and can include discrete digital or analog circuit elements or electronics, or combinations thereof. In an aspect, the controller includes one or more ASICs having a plurality of pre-defined logic components. In an aspect, the controller includes one or more FPGA having a plurality of programmable logic commands.

Controller 235 further includes circuitry 245. In an aspect, circuitry 245 includes circuitry configured to execute one or more instructions for operating components of the walking aid, e.g., the at least one first sensor, the at least one second sensor, the transmission unit, and the two or more extendable and retractable load-bearing feet. In an aspect, circuitry 245 includes circuitry configured to execute one or more instructions for operating any or all other components incorporated into the walking aid, e.g., a user-activated control mechanism, a user interface, a receiver, at least one third sensor, an actuator, a power source, a mechanical energy harvester, and/or a warning system. In an aspect, the controller includes circuitry to execute one or more instructions for receiving information regarding the detected parameter of the walking surface from the at least one first sensor and circuitry to execute one or more instructions for actuating at least one of the two or more extendable and retractable load-bearing feet in response to the information regarding the detected parameter of the walking surface.

Controller 235 includes circuitry 245 including actuation circuitry configured to actuate at least one of the two or more extendable and retractable load-bearing feet in response to the information regarding the detected parameter of the walking surface. In some embodiments, as shown in block 750, the actuation circuitry is configured to actuate at least one of the two or more extendable and retractable load-bearing feet in response to the information regarding the detected parameter of the walking surface before the at least one of the two or more extendable and retractable load-bearing feet contacts the walking surface. For example, the controller can receive information from the at least one first sensor, e.g., a camera or proximity sensor, regarding a parameter of the walking surface, e.g., a curb edge or break in the sidewalk, and the actuation circuitry can actuate at least one of the two or more extendable and retractable load-bearing feet before the at least one of the two or more extendable and retractable load-bearing feet contacts the walking surface. In an aspect, the actuation circuitry of block 750 is configured to execute one or more instructions for actuating at least one of the two or more extendable and retractable load-bearing feet in response to the information regarding the detected parameter of the walking surface before the at least one of the two or more extendable and retractable load-bearing feet contacts the walking surface.

In an aspect, circuitry 245 of controller 235 includes actuation circuitry configured to actuate extension or retraction of at least one of the two or more extendable and retractable load-bearing feet in response to the information regarding the detected parameter of the walking surface before the at least one of the two or more extendable and retractable load-bearing feet contacts the walking surface. For example, the controller can receive information from the at least one first sensor, e.g., a camera or proximity sensor, regarding a parameter of the walking surface, e.g., a curb edge or break in the sidewalk, and the actuation circuitry can actuate extension or retraction of at least one of the two or more extendable and retractable load-bearing feet before the at least one of the two or more extendable and retractable load-bearing feet contacts the walking surface. In an aspect, the actuation circuitry is configured to execute one or more instructions for actuating extension or retraction of at least one of the two or more extendable and retractable load-bearing feet in response to the information regarding the detected parameter of the walking surface before the at least one of the two or more extendable and retractable load-bearing feet contacts the walking surface.

In an aspect, circuitry 245 of controller 235 includes actuation circuitry configured to independently and automatically actuate the two or more extendable and retractable load-bearing feet. For example, the controller can receive information from the at least one first sensor, e.g., a proximity sensor, regarding a parameter of the walking surface, e.g., a stepped walking surface, and the actuation circuitry can independently and automatically actuate the two or more extendable and retractable load-bearing feet to create a stable support for the user. For example, the controller can include actuation circuitry configured to independently and automatically actuate each of the two of more extendable and retractable load-bearing feet. For example, the controller can include actuation circuitry configured to actuate one of the two or more extendable and retractable load-bearing feet independently of each of the other two or more extendable and retractable load-bearing feet. In an aspect, the actuation circuitry is configured to execute one or more instructions for independently and automatically actuating the two or more extendable and retractable load-bearing feet. In some embodiments, as shown in block 760, the actuation circuitry is configured to independently and automatically actuate the two or more extendable and retractable load-bearing feet to a configuration based on a position of the walking aid relative to the walking surface. For example, the controller can receive information from the at least one first sensor, e.g., a proximity sensor, regarding the position of the walking aid relative to the walking surface, e.g., height and distance away from a step associated with a stepped walking surface, and the actuation circuitry can independently and automatically actuate the two or more extendable and retractable load-bearing feet to create a stable support for the user. In an aspect, the actuation circuitry of block 760 is configured to execute one or more instructions for independently and automatically actuating the two or more extendable and retractable load-bearing feet to a configuration based on a position of the walking aid relative to the walking surface.

In some embodiments, as shown in block 770, the actuation circuitry is configured to independently and automatically actuate the two or more extendable and retractable load-bearing feet to a configuration based on a position of variance of the walking aid from vertical. For example, the controller can receive information from at least one accelerometer regarding a tilt or inclination of the walking aid relative to the walking surface and the actuation circuitry can independently and automatically actuate the two or more extendable and retractable load-bearing feet to create stable support for the user. In an aspect, the actuation circuitry of block 770 is configured to execute one or more instructions for independently and automatically actuating the two or more extendable and retractable load-bearing feet to a configuration based on a position of variance of the walking aid from vertical.

Returning to FIG. 7, in some embodiments, walking aid 200 includes user-activated control mechanism 775. In an aspect, a walking aid includes a user-activated control mechanism to control extension and retraction of the two or more extendable and retractable load-bearing feet. In an aspect, the user-activated control mechanism 775 is a voice-activated control mechanism 780. For example, the walking aid can include a microphone and circuitry configured to receive, process, and transmit a voice command to the controller. For example, the voice commands can include “on,” “off,” “extend,” “retract,” and the like. In an aspect, the user-activated control mechanism 775 is a manually activated control mechanism 785. For example, the walking aid can include one or more buttons or other pressure sensitive pads and circuitry configured to receive and process a manual activation command, e.g., pushing of a button. In an aspect, at least a portion of the user-activated control mechanism is incorporated into the hand grip disposed in proximity to the first end of the linear rod. For example, the hand grip can include a microphone for receiving a voice command. For example, the hand grip can include one or more pressure-sensitive buttons or pads for sending a manual activation command.

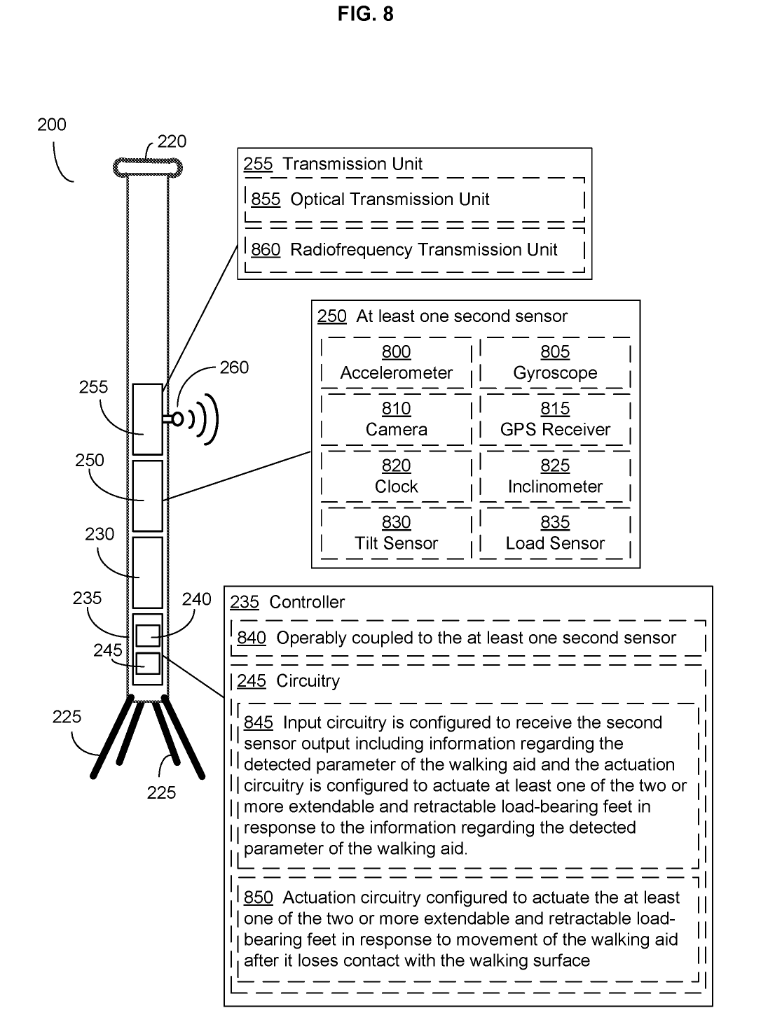

FIG. 8 illustrates further aspects of a walking aid. In some embodiments, walking aid 200 includes at least one second sensor 250 configured to detect a parameter of the walking aid 200 and to transform the detected parameter of the walking aid 200 into a second sensor output. In an aspect, the parameter of the walking aid comprises at least one of movement, speed, location, tip motion of the walking aid, position of walking aid relative to the walking surface, or tilt angle of the walking aid relative to the walking surface. In an aspect, the at least one second sensor 250 measures the movement of the walking aid in three-dimensional space. In an aspect, the at least one second sensor 250 measured the movement of the walking aid relative to a walking surface. In an aspect, the at least one second sensor 250 measures the movement of the walking aid relative to the user.

In an aspect, the at least one second sensor 250 comprises at least one accelerometer 800. For example, the at least one second sensor can include at least one accelerometer configured to measure the force of acceleration of the walking aid and associated sense movement, speed and direction. In an aspect, the at least one second sensor 250 comprises at least one gyroscope 805. For example, the at least one second sensor can include at least one gyroscope configured to measure orientation and rotation of the walking aid. In combination with an accelerometer, the gyroscope can provide information regarding the movement of the walking aid in three-dimensional space. In an aspect, the at least one second sensor 250 comprises at least one camera 810. For example, the at least one second sensor can include at least one digital camera aimed to capture images of the user, the walking surface, or a combination of both to assess the movement of the user and/or the movement of the walking aid relative to the walking surface. In an aspect, the at least one second sensor 250 comprises a GPS receiver 815. For example, the at least one second sensor can include a GPS receiver for determining a location and velocity of the walking aid at a specific point in time that is an L-band radio processor capable of processing signals broadcasted from GPS satellites. In an aspect, the at least one second sensor 250 comprises a local positioning systems. For example, the at least one second sensor can include at least one receiver configured to receive a signal from one or more beacons in a localized indoor area (e.g., a residence, assisted living facility, healthcare facility, or commercial facility) or an outdoor area. In an aspect, the at least one second sensor 250 comprises at least one clock 820. In an aspect, the clock provides absolute time, e.g., the current time. For example a clock function can be provided by signals from GPS satellites processed by a GPS receiver. In an aspect, the clock provides running or relative time. For example, the clock may include a start/stop function linked to activation of another sensor, e.g., an accelerometer or gyroscope. In an aspect, the at least one second sensor 250 includes at least one of an inclinometer 825, a tilt sensor 830, or a load sensor 835, non-limiting examples of which have been described above herein. In an aspect, the at least one second sensor includes at least one of a tactile sensor, a whisker sensor, a proximity sensor, and/or a pressure sensor.

In an aspect, the at least one second sensor provides information regarding a parameter of the walking aid, e.g., movement, relative to a walking surface and/or a user. In an aspect, the at least one second sensor provides an indication of frequency and duration of use of the walking aid, as indicated by accelerometers, motion detectors, and clocks. In an aspect, the at least one second sensor provides an indication of where a user is going with the walking aid, as indicated by a GPS receiver associated with the walking aid or access to a local positioning system with localized beacons. In an aspect, the at least one second sensor provides an indication as to a path (e.g., a straight versus weaving path) a user is taking with the walking aid. In an aspect, the at least one second sensor provides an indication as to how fast a user is traveling with the walking aid, as indicated by a speedometer sensor. In an aspect, the at least one second sensor provides an indication of the quality of the walking, e.g., is the user weaving, shuffling, leaning to one side, or other.

In an aspect, the at least one second sensor 250 is configured to determine movement of the walking aid before, during, and after it loses contact with the walking surface. For example, an accelerometer in combination with a pressure sensor can be used to detect when the walking aid loses contact with the walking surface and what the movement of the walking aid looks like after losing contact. In an aspect, a tilt sensor or inclinometer in combination with accelerometers can measure the pitch and roll of the walking aid relative to the walking surface before, during, and after the walking aid loses contact with the walking surface. In an aspect, a combination of two or more sensors is used to measure the height of the walking aid. For example, a combination of 3D gyroscopes, 3D accelerometers, 3D magnetometers, and a barometric altimeter can be used as a height sensor. See, e.g., Tanigawa et al. (2008) “Drift-free dynamic height sensor using MEMS IMU aided by MEMS pressure sensor” Proceedings of the 5^{th} Workshop on Positioning, Navigation, and Communication 2008, pp. 191-196, which is incorporated herein by reference.

In an aspect, at least one second sensor 250 is configured to detect tip motion at the first end or the second end of the linear rod after initial contact with the walking surface. For example, a 2-axis inclinometer with accelerometers can be used to measure a tipping motion, tilt, or variance from vertical of the walking aid relative to a walking surface. Non-limiting examples of inclinometers and accelerometers have been described above herein.

Returning to FIG. 8, walking aid 200 further includes transmission unit 255 operably coupled to the at least one second sensor 250, transmission unit 255 including an antenna 260 and circuitry configured to transmit the second sensor output to a computing device. A “transmission unit,” as used herein, can be one or more of a variety of units that are configured to send and/or receive signals, such as signals carried as electromagnetic, radio, acoustic, sonic, or optical waves. A transmission unit generally includes at least one antenna and associated circuitry. A transmission unit can be operably connected to controller 235 and/or can include its own processor and/or memory component. A transmission unit can be operably connected to an energy source, such as a battery. A transmission unit can include an energy harvesting unit, such as a unit configured to obtain energy from electromagnetic waves. A transmission unit can include a transponder utilizing electromagnetic waves, for example as described in “Fundamental Operating Principles,” in Chapter 3 of the RFID Handbook: Fundamentals and Applications in Contactless Smart Cards and Identification, Klaus Finkenzeller, John Wiley & Sons, (2003), which is incorporated herein by reference. In an aspect, the transmission unit communicates within a wireless personal area network, e.g., Bluetooth, wireless USB, WiFi, or ZigBee.

In an aspect, the transmission unit 255 comprises a radiofrequency transmission unit 860. A transmission unit can include an oscillator and encoder configured to generate a programmable pulse position-modulated signal in the radio frequency range (see, e.g., U.S. Pat. No. 4,384,288, which is incorporated herein by reference). A transmission unit can include a radio frequency identification device (RFID), which can be a passive RFID device, a semi-passive RFID device, or an active RFID device, depending on the embodiment (see, e.g., Chawla & Ha, “An Overview of Passive RFID,” IEEE Applications and Practice, 11-17 (September 2007), which is incorporated herein by reference). A transmission unit including an RFID device can be configured to transmit signals in the UHF standard range. A transmission unit can include a battery-assisted passive RFID device, such as sold by Alien Technology®, Morgan Hill, Calif. In an aspect, the transmission unit 255 comprises an optical transmission unit 855. For example, the optical transmission unit can include an infrared transmitting diode. A transmission unit can include a hybrid backscatter system configured to function in an RFID, IEEE 802.11x standard and Bluetooth system. A transmission unit can include a near field communication (NFC) device. A transmission unit can include a Wireless Identification and Sensing Platform (WISP) device. A transmission unit can be operably coupled to a data storage unit.

In an aspect, the transmission unit 255 includes circuitry configured to transmit the second sensor output to a computing device. For example, the transmission unit of the walking aid can be configured to sync with an external device, e.g., a smart phone, to transfer information, e.g., the second sensor output including the detected parameter of the walking aid. In an aspect, the transmission unit is configured to transmit the second sensor output including the detected parameter of the walking aid to a dedicated handheld device. For example, the transmission unit includes circuitry configured to transmit the second sensor output including the detected parameter of the walking aid to a dedicated handheld device specifically designed for use with the walking aid. For example, a dedicated handheld device can include a transmission unit and antenna for communicating with the walking aid, a user interface, e.g., a display, microphone, or haptic interface, for displaying and/or notifying a user during a walking event, and a computing component to display and save information received from the walking aid. In an aspect, the transmission unit includes circuitry configured to transmit the second sensor output to a mobile communication device. For example, the transmission unit can include circuitry configured to transmit the second sensor output including the detected parameter of the walking aid to a mobile communication device, e.g., a cellular or smart phone. For example, the transmission unit including the antenna can be connected (e.g., “synced”) through a wireless radiofrequency communication link, e.g., Bluetooth or WiFi, to a smart phone. In an aspect, the mobile communication device includes a program, set of instructions, and/or an application configured to receive information from the walking aid, process the received information to determine features of a user's gait, and display the received information and/or the determined features of a user's gait. In an aspect, the transmission unit includes circuitry configured to transmit the second sensor output including the detected parameter of the walking aid to a computing device. For example, the transmission unit can transmit the second sensor output including the detected parameter of the walking aid to a computing device, e.g., a tablet, a laptop, or tabletop computing device. In an aspect, the transmission unit includes circuitry configured to transmit the second sensor output including the detected parameter of the walking aid to a cloud computing device.

In an aspect, the transmission unit includes circuitry configured to communicate with a communication device, such as one or more of a mobile communication device and a computer system including, but not limited to, mobile computing devices (e.g., hand-held portable computers, Personal Digital Assistants (PDAs), laptop computers, netbook computers, tablet computers, and so forth), mobile telephone devices (e.g., cellular telephones and smartphones), devices that include functionalities associated with smartphones and tablet computers (e.g., phablets), portable game devices, portable media layers, multimedia devices, satellite navigation devices (e.g., Global Positioning System (GPS) navigation devices), e-book reader devices (eReaders), Smart Television (TV) devices, surface computing devices (e.g., table top computers), Personal Computer (PC) devices, and other devices that employ touch-based human interfaces. In an aspect, the computing device is associated with another piece of equipment associated with a patient care room in a hospital or other medical facility or lactation clinic.

In an aspect, the transmission unit includes circuitry configured to transmit the second sensor output including the detected parameter of the walking aid to a network. In an aspect, the transmission unit is configured to communicate with a health provider network. For example, the reporting device can be configured to communicate directly with a network associated with a subject's healthcare provider, e.g., a hospital, a clinic, independent and/or assisted living facility, medical facility, or physician's office. For example, the reporting device can be configured to communicate directly with the subject's electronic medical file or health record.

In an aspect, controller 235 is operably coupled to the at least one second sensor 250, as shown in block 840 and the input circuitry is configured to receive the second sensor output including information regarding the detected parameter of the walking aid 200 and the actuation circuitry is configured to actuate at least one of the two or more extendable and retractable load-bearing feet 225 in response to the information regarding the detected parameter of the walking aid 200, as shown in block 845. In some embodiments, the movement of the walking aid relative to the user and the walking surface can inform actuation of the extendable and retractable feet. For example, the controller can include circuitry to actuate at least one of the two or more extendable and retractable load-bearing feet in response to detecting a substantial tilt in the walking aid relative to the walking surface. In an aspect, the input circuitry is configured to execute one or more instructions for receiving the second sensor output including the information regarding the detected parameter of the walking aid 200 from the at least one second sensor 250 and the actuation circuitry is configured to execute one or more instructions for actuating at least one of the two or more extendable and retractable load-bearing feet 225 in response to the information regarding the detected parameter of the walking aid 200.

In some embodiments, circuitry 245 of controller 235 includes actuation circuitry configured to actuate the at least one of the two or more extendable and retractable load-bearing feet 225 in response to movement of the walking aid 200 after it loses contact with the walking surface, as shown in block 850. For example, the actuation circuitry can be configured to actuate at least one of the two or more extendable and retractable load-bearing feet based on information from one or more accelerometers regarding movement of the walking aid after it loses contact with the walking surface. In an aspect, the actuation circuitry is configured to execute one or more instructions for actuating the at least one of the two or more extendable and retractable load-bearing feet 225 in response to movement of the walking aid 200 after it loses contact with the walking surface.

In an aspect, controller 235 includes circuitry 245 configured to actuate extension or retraction of the at least one of the two or more extendable and retractable load-bearing feet 225 in response to movement of the walking aid 200 after it loses contact with the walking surface. For example, the circuitry can be configured to extend or retract at least one of the two or more extendable and retractable load-bearing feet based on information from one or more accelerometers regarding movement of the walking aid after it loses contact with the walking surface. In an aspect, the actuation circuitry is configured to execute one or more instructions for actuating extension or retraction of the at least one of the two or more extendable and retractable load-bearing feet 225 in response to movement of the walking aid 200 after it loses contact with the walking surface.

In an aspect, controller 235 includes circuitry 245 including actuation circuitry configured to actuate the at least one of the two or more extendable and retractable load-bearing feet 225 to orient said at least one of the two or more extendable and retractable load-bearing feet 225 relative to an orientation of the walking aid 200. For example, the actuation circuitry can be configured to orient the two or more extendable and retractable load-bearing feet at an angle relative to the orientation of the walk aid. For example, the actuation circuitry can be configured to orient the two or more extendable and retractable load-bearing feet relative to the walking surface when the walking aid is oriented at an angle (e.g., tilted) relative to the walking surface to create a stable support for the user. In an aspect, the actuation circuitry is configured to execute one or more instructions for actuating the at least one of the two or more extendable and retractable load-bearing feet 225 to orient said at least one of the two or more extendable and retractable load-bearing feet 225 relative to an orientation of the walking aid 200.

In some embodiments, walking aid 200 includes an actuator operably coupled to the controller and the actuation circuitry and configured to receive instructions from the actuation circuitry to actuate the two or more extendable and retractable load-bearing feet. In an aspect, the actuator produces a rotational motion (e.g., rotating at least a portion of an extendable and retractable load-bearing foot around a pivot point). In an aspect, the actuator produces a linear motion (e.g., extending and retracting the two or more telescoping segments of the extendable and retractable load-bearing foot). In an aspect, the actuator includes at least one of a spring, a motor, a pneumatic actuator, or a hydraulic actuator. In an aspect, the actuator includes at least one of a linear actuator, a solenoid, a muscle wire, or a mechanical actuator. In an aspect, the actuator comprises a combination of a spring, a motor, a pneumatic actuator, a hydraulic actuator, a linear actuator, a solenoid, and/or a muscle wire. For example, a pneumatic actuator can be combined with a spring to extend and retract one or more of the extendable and retractable load-bearing feet.

In an aspect, the actuator includes at least one motor including circuitry configured to extend and retract the extendable and retractable load-bearing feet. In an aspect, the motor includes at least one of a DC motor, a geared DC motor, a servo motor, a stepper motor, or a geared stepper motor. In an aspect, the at least one motor is an electric motor. In an aspect, the motor is a rotary shaft motor, such as a conventional DC, pulse, or AC motor. In an aspect, the motor can include a brush DC motor. In an aspect, the motor can include a DC servo. In an aspect, the motor can include a rotary piezoelectric motor. Other non-limiting examples of motors for use in actuating the extendable and retractable load-bearing feet include a stepper control motor, a brushless DC commutated control motor, or a universal motor. For example, a stepper motor or servo can be used to extend or retract the extendable and retractable load-bearing feet that include two or more telescoping segments. For example, a motorized threaded rod can be used to extend or retract the extendable and retractable load-bearing feet. In general, motors for use in small electronics or hand-held devices are known in the art and available from commercial sources.

In an aspect, the actuator includes a pneumatic actuator. See, e.g., Granosik & Borenstein (2005) “Pneumatic actuators for serpentine robot” 8^{th} International Conference on Walking and Climbing Robots (CLAWAR), London, U.K., 12-15 Sep. 2005, pp. 719-726, which are incorporated herein by reference. Pneumatic actuators are available from commercial sources (from, e.g., Bimba Manufacturing, University Park, Ill.; ASCO Numatics, Florham Park, N.J.).

In an aspect, the actuator includes a thermal- or magnetic-responsive actuator. For example, the actuator can include a shape memory material, e.g., shape memory alloys responsive to heat and/or magnetic shape-memory alloys responsive to a magnetic field. For example, the actuator can include a spring formed from a shape memory material. See, e.g., U.S. Pat. No. 7,104,056 to Taya et al. titled “Design of ferromagnetic shape memory allow composites and actuators incorporating such materials;” and U.S. Pat. No. 8,586,176 to Taya & Liang titled “Shape memory alloy fibers and shape memory polymer fibers and films and their composites for reversible shape changes,” which are incorporated herein by reference.

In an aspect, actuator includes a mechanical actuator configured to convert rotary motion into linear motion. For example, the mechanical actuator can use one or more of gears, rails, pulleys, chains, or other devices to operate. For example, extension and retraction of the two or more extendable and retractable load-bearing feet can be actuated using a series of gears and pulleys operably linked to one or more motors.

In an aspect, the walking aid includes a power source for powering any or all components of the walking aid. In an aspect, the walking aid includes one or more disposable batteries, e.g., cells, buttons, thin-film batteries, or microbatteries. For example, the components of the walking aid can be powered by a conventional battery, e.g., a disposable 9 volt battery. Non-limiting examples of disposable batteries include zinc-carbon, alkaline, lithium, zinc-chloride, zinc-air, or silver-oxide batteries. In an aspect, the one or more batteries include one or more rechargeable batteries. For example, the components of the walking aid can be powered by one or more rechargeable lithium-ion batteries. Non-limiting examples of rechargeable batteries include nickel-cadmium, nickel-zinc, nickel metal hydride, silver-zinc, or lithium ion.

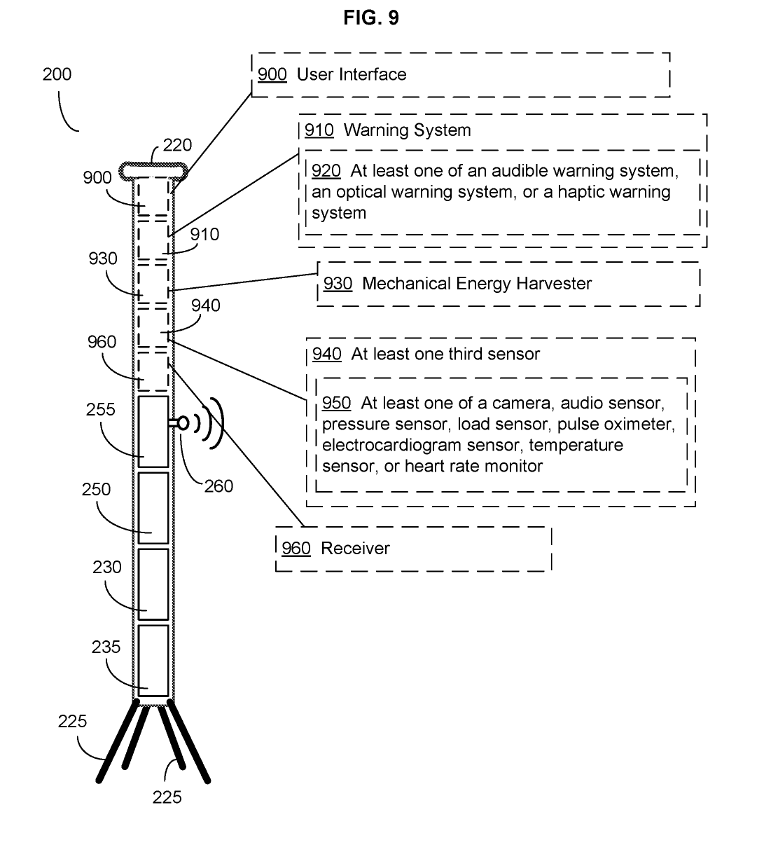

FIG. 9 illustrates further aspects of a walking aid. In some embodiments, walking aid 200 further includes a user interface 900 operably coupled to the controller 235. The user interface can include one or more input components and/or output components for use by a user to interface with the walking aid. In an aspect, the user interface can include an input component (e.g., a microphone, button, or switch) for controlling one or more functions of the walking aid. For example, the user interface can include an input component that is part of a user-activated control mechanism to control extension and retraction of the two or more extendable and retractable load-bearing feet. Non-limiting examples of input components include buttons, switches, triggers, keypads, touch-screens, microphones, dials, or the like. In an aspect, the user interface can include an output component for providing information to a user. For example, an output component, e.g., a flashing light emitting diode (LED), can be used as part of a warning system configured to alert a user. Non-limiting examples of output components include audio speakers, light emitting diodes or other light generating devices, or a haptic device. In some embodiments, user interface 900 is part of a user-activated control mechanism to control extension and retraction of the two or more extendable and retractable load-bearing feet 225. For example, one or more buttons on the hand grip can be used to manually control extension and retraction of the two or more extendable and retractable load-bearing feet. In some embodiments, the user interface 900 is part of a warning system 910 operably coupled to the controller 235 and configured to alert a user. For example, a light emitting diode on the hand grip can be used to alert a user to a hazard associated with the walking surface, e.g., a crack in a sidewalk or the edge of a curb. In some embodiments, user interface 900 includes a display. For example, the user interface can include an OLED, LCD, or touch screen display with a screen intended to display information. The information can include, but is not limited to, information regarding a property of the walking surface, information regarding a property of the walking aid, a physiological parameter of the user, an alert or warning, a map, information from another device (e.g., a wearable device worn by the user). In an aspect, the user interface 900 is associated with the hand grip 220 and/or the linear rod 205 of walking aid 200.

In some embodiments, walking aid 200 includes a warning system 910 operably coupled to the controller 235 and configured to alert a user, wherein the warning system 910 comprises at least one of an audible warning system, an optical warning system, or a haptic warning system 920. For example, the walking aid can include a warning system that alerts a user to a dangerous walking surface or situation. For example, the walking aid can include a warning system that alerts a user to the dangers of a slippery surface, e.g., an oily, icy, or water-covered surface). For example, the walking aid can include a warning system that alerts a user to dangers associated with a specific friction coefficient, e.g., too little or too much, that might create a dangerous walking situation. For example, the walking aid can include a warning system that alerts a user to the dangers of a slope in the walking surface, e.g., an incline or a decline of a percent grade that might be of difficulty for the user. For example, the walking aid can include a warning system that alerts a user to the proximity of an edge, e.g., an edge associated with a curb or a step. For example, the walking aid can include a warning system that alerts a user to poor load-bearing capability of a surface, e.g., the poor load-bearing capability of deep sand or thick grass. For example, the walking aid can include a warning system that provides feedback in response to a calculated gait value.

In an aspect, warning system 910 is at least one of an audible warning system, an optical warning system, or a haptic warning system 920. In an aspect, the warning system includes a speaker, a soundcard, and circuitry configured to broadcast an audible alert in response to receiving a warning signal from the controller. For example, the warning system can include an audible alert, e.g., a beeping sound or a warning voice. In an aspect, the warning system includes one or more light sources, and circuitry configured to emit an optical alert in response to receiving a warning signal from the controller. For example, the warning system can include an optical alert, e.g., one or more flashing red LEDs. In an aspect, the warning system includes a vibrator and circuitry configured to vibrate at least a portion of the walking aid in response to receiving a warning signal from the controller. For example, the hand grip of the walking aid can include a haptic device, e.g., a vibrator, which vibrates the hand grip in response to a warning signal from the controller.

In an aspect, warning system 910 is incorporated into the hand grip 220 of walking aid 200. In an aspect, at least one of an audible warning system, an optical warning system, or a haptic warning system 920 is incorporated into hand grip 220. For example, the warning system can include one or more lights, e.g., LEDs, incorporated into the hand grip that flash on/off to alert the user. For example, the warning system can include a speaker incorporated into the hand grip that emits an audible sound, e.g., a beeping sound, to alert the user. For example, the warning system can include a haptic vibration device that vibrates the hand grip of the walking device to alert the user. Alternatively, the warning system can be incorporated into one or more other parts of the walking aid, e.g., into the linear rod or into at least one of the two or more extendable and retractable load bearing feet.

In some embodiments, walking aid 200 further includes a mechanical energy harvester 930 configured to store electrical or mechanical energy to power actuation of the two or more extendable and retractable load-bearing feet 225. In an aspect, the mechanical energy harvester stores mechanical energy generated when the walking aid periodically contacts the walking surface. For example, the intermittent mechanical energy generated when the walking aid contacts the walking surface can be converted into electrical energy through at least one of piezoelectric conversion, electro-active polymers (EAP) conversion, or electromagnetic conversion. For example, the mechanical energy harvester can include one or more piezoelectric materials, dielectric elastomers, and/or rotator generators. See, e.g., Yusuf et al. (2013) “Mechanical energy harvesting devices for low frequency applications: Revisited,” ARPN J Eng. App. Sci. 8:504-512, which is incorporated herein by reference. In an aspect, the mechanical energy harvester stores electrical or mechanical energy for powering one or more components of the walking aid. In an aspect, the mechanical energy harvester stores electrical or mechanical energy for powering an actuator, e.g., spring, a motor, a pneumatic actuator, a hydraulic actuator, or solenoid, to power actuation of the two or more extendable and retractable load-bearing feet 225. In an aspect, power from a mechanical energy harvester is combined with another power source, e.g., a standard battery, to power the components of the walking aid.

In some embodiments, a walking aid such as described herein may further include additional sensors for monitoring a physiological condition of a user. In an aspect, walking aid 200 includes at least one third sensor 940 operably coupled to the transmission unit 255 and including circuitry configured to detect a physiological parameter of a user and to transform the detected physiological parameter of the user into a third sensor output, wherein the transmission unit 255 includes circuitry configured to transmit the third sensor output to the computing device. In some embodiments, the at least one third sensor 940 is incorporated into the hand grip 220 of walking aid 200. For example, the walking aid can include one or more physiological sensor incorporated into the hand grip. In an aspect, the at least one third sensor 940 includes at least one camera, audio sensor, pressure sensor, load sensor, pulse oximeter, electrocardiogram sensor, temperature sensor, or heart rate monitor, as shown in block 950 of FIG. 9. For example, the at least one third sensor can include a camera (e.g., a digital camera) positioned to capture images of a portion of the user. The camera can be positioned to image the user's legs to monitor gait. The camera can be positioned to image the user's face or other area of exposed skin to assess coloring (e.g., flushed, ashen, purple, jaundiced, etc.). For example, the at least one third sensor can include an audio sensor, e.g., a microphone, to pick up sounds associated with the user and his/her use of the walking aid. In an aspect, the audio sensor is configured to detect speech. For example, the audio sensor can include a microphone configured to capture speech, both speech quality (e.g., speech patterns, frequency, fluency, loudness/softness, slurring) and speech content (e.g., social communication, information processing, attention/concentration, memory, reasoning, problem-solving, literal interpretation, altered turn-taking skills, interrupting, reduced range of topics, fixating on certain subjects or topics, talking in sexually explicit manner, swearing at inappropriate times, or inability to convey information in organized manner) as a means of monitoring a user's mental or medical state. In an aspect, the audio sensor is configured to detect non-speech sounds. For example, the audio sensor can be configured to detect breathing patterns (e.g., shortness of breath or wheezing with limited exertion). For example, the audio sensor can include a microphone configured to capture sounds of the user's feet (e.g., foot or shoe-strike including shuffling) while walking with the walking aid. For example, the at least one third sensor can include a pressure sensor, e.g., in the hand grip, configured to measure the grip strength of a user. For example, the at least one third sensor can include a load sensor configured to measure how much of the user's body weight is being supported by the walking aid. For example, the at least one third sensor can include a pulse oximeter configured to measure the blood oxygenation or oxygen saturation of the user's blood. For example, the at least one third sensor can include a thermometer for monitoring the body temperature of the user. For example, the at least one third sensor can include a heart rate monitor attached to the hand grip of the walking aid and configured to monitor the heart rate of a user while using the walking aid.

In some embodiments, the at least one third sensor 940 of walking aid 200 is configured to transmit a signal including information regarding the detected physiological parameter of the user to at least one of the user interface 900 or the warning system 910 to alert the user as to the detected physiological parameter. In some embodiments, the transmitted signal merely provides an alert to the user (e.g., a blinking light or an audible beeping) indicative of a detected physiological parameter. This might be of particular use if the detected physiological parameter (e.g., temperature, heart rate, or oxygenation) is outside a normal range. In some embodiments, the transmitted signal carries a number or text (e.g., a specific value associated with the detected physiological parameter) for display or audio reporting by a user interface or warning system.

Returning to FIG. 9, in some embodiments, walking aid 200 includes at least one receiver 960. In some embodiments, the walking aid includes at least one receiver including circuitry configured to receive a signal from the computing device. For example, the walking aid can include a receiver for receiving signals from at least one of a mobile communication device, a dedicated hand-held device, a computer (e.g., a laptop, tablet, or desktop computer), a network, a server, or other device or system. In some embodiments, the walking aid includes at least one receiver including circuitry configured to receive a signal from a wearable device. For example, the walking aid can include a receiver configured to receive signals transmitted from a fitness monitor, heart rate monitor, pedometer, or similar type of device. In some embodiments, the receiver includes circuitry to receive a signal transmitted from one or more wearable inertial sensors including accelerometers and/or gyroscopes placed on the feet, knees, and/or thighs. In some embodiments, the receiver includes circuitry configured to receive a signal transmitted from a wearable electromyography sensor worn on the leg of the user to provide muscle use information during ambulation. The information received by the receiver from a wearable device can be added to or correlated with information incorporated into the first and second sensor outputs. The information received can be added to or correlated with information incorporated in a third sensor output including a detected physiological parameter of the user.

In an aspect, receiver 960 is an optical receiver including circuitry configured to receive an optical signal from a remote optical signal generator. In an aspect, the optical receiver includes a photodetector. Non-limiting examples of photodetectors include photodiodes, metal-semiconductor-metal (MSM) photodetectors, phototransistors, photoresistors, photomultipliers, pyroelectric photodetectors, and thermal detectors. In an aspect, the at least one receiver comprises an array of photodetectors. Photodetectors are available from commercial sources (from, e.g., AC Photonics, Santa Clara, Calif.; Electro-Optics Technology, Inc., Traverse City, Mich.; and OSI Laser Diode, Inc., Edison, N.J.).

In an aspect, receiver 960 can include a radiofrequency receiver including circuitry configured to receive a radiofrequency signal from an external radiofrequency signal generator. In an aspect, the radiofrequency signal receiver is tuned to receive a specific frequency. In an aspect, the radiofrequency signal receiver includes a radiofrequency module, e.g., a superheterodyne receiver or a super-regenerative receiver. In an aspect, the radiofrequency module is part of an electronic circuit and can include a WLAN module, a Zigbee module, a Bluetooth module, a GPS module, an RFID module, or a UHF module (from, e.g., Future Electronics, Pointe Claire, Quebec, Canada).

In an aspect, receiver 960 includes circuitry configured to receive at least one of a radio signal, a magnetic signal, an acoustic signal, an electromagnetic signal, or an optical signal. The receiver can include at least one of an electromagnetic signal receiver, a magnetic signal receiver, a microwave signal receiver, an acoustic signal receiver, an optical signal receiver, or other receiver types. In an aspect, the receiver includes at least one antenna configured to intercept signals emitted from an external device (e.g., a computing device, a wearable device, and/or a remote signaling device). In an aspect, the receiver includes at least one filter, e.g., an electronic filter, a frequency filter, or a bandwidth filter, to separate specific signals from the external device from other signals in the environment. In an aspect, the receiver includes a digital signal processor. In an aspect, receiver 960 and transmitter 255 are part of a transceiver or similar type of component capable of both transmitting and receiving signals.

In some embodiments, the walking aid includes at least one receiver including circuitry configured to receive a signal from a remote signaling device and to transform the received signal from the remote signaling device into information regarding a location of the walking aid in an environment. In an aspect, receiver 960 is configured to receive a signal from a remote signaling device as a user approaches an environment. In an aspect, the receiver 960 and/or an operably coupled controller 235 is configured to transform the received signal into information regarding a location of walking aid 200 in the environment, e.g., approaching a stepped surface. In some embodiments, the actuation circuitry of controller 235 is configured to actuate at least one of the two or more extendable and retractable load-bearing feet 225 based on the information regarding the location of the walking aid 200 in the environment.

Described herein is a system including a walking aid and a computing device. In some embodiments, a system includes a walking aid including a linear rod having a first end and a second end; a hand grip disposed in proximity to the first end of the linear rod; two or more extendable and retractable load-bearing feet at the second end of the linear rod, at least one of the two or more extendable and retractable load-bearing feet including two or more telescoping segments; at least one first sensor including circuitry configured to detect a parameter of a walking surface in proximity to the second end of the linear rod and to transform the detected parameter of the walking surface into a first sensor output; a controller including a microprocessor and circuitry, the controller operably coupled to the two or more extendable and retractable load-bearing feet and the at least one first sensor and including input circuitry configured to receive the first sensor output including information regarding the detected parameter of the walking surface, and actuation circuitry configured to actuate at least one of the two or more extendable and retractable load-bearing feet in response to the received information regarding the detected parameter of the walking surface; at least one second sensor including circuitry configured to detect a parameter of the walking aid and to transform the detected parameter of the walking aid into a second sensor output; and a transmission unit operably coupled to the at least one second sensor, the transmission unit including an antenna and circuitry configured to transmit the second sensor output to a computing device; and the computing device including a processor and circuitry including input circuitry configured to receive the second sensor output including information regarding the detected parameter of the walking aid; and calculation circuitry configured to calculate a gait value based on the received information regarding the detected parameter of the walking aid.

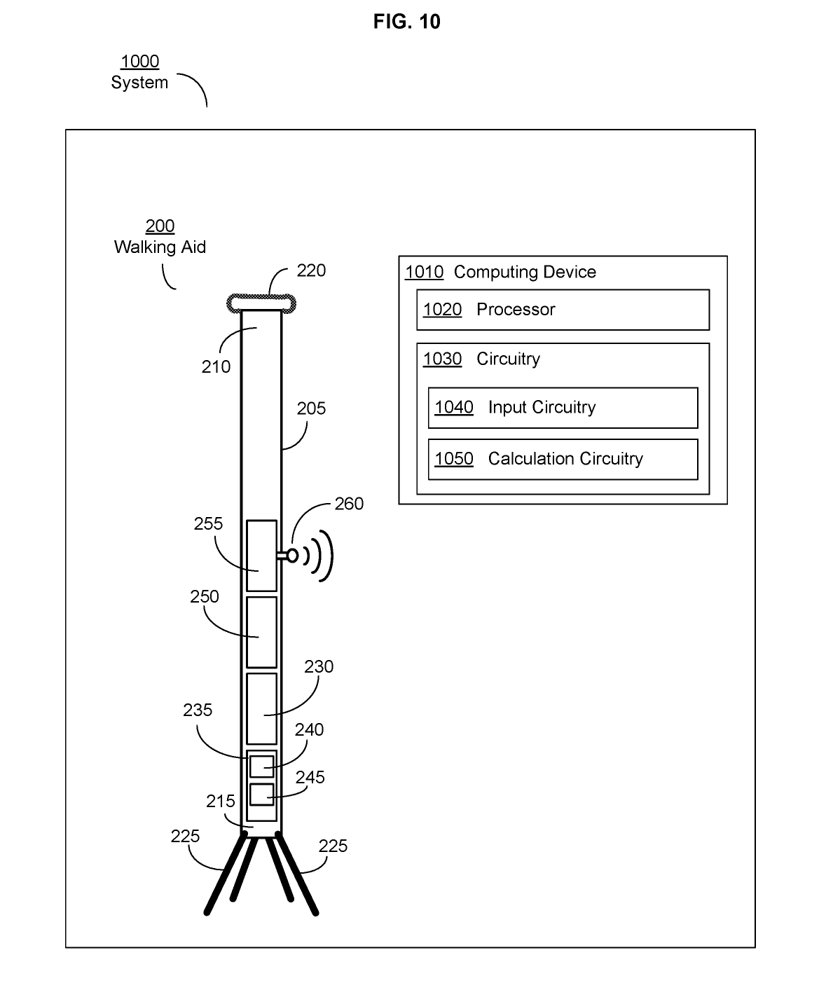

FIG. 10 illustrates non-limiting aspects of a system including a walking aid and a computing device. System 1000 includes walking aid 200 and computing device 1010. Walking aid 200 of system 1000 includes a linear rod 205 having a first end 210 and a second end 215 and hand grip 220 disposed in proximity to the first end 210 of linear rod 205. Walking aid 200 further includes two or more extendable and retractable load-bearing feet 225 at the second end 215 of linear rod 205, at least one of the two or more extendable and retractable load-bearing feet 225 including two or more telescoping segments. In an aspect, at least one of the two or more extendable and retractable load-bearing feet 225 of walking aid 200 has a pivot at a connection to the linear rod 205. In some embodiments, at least one of the two or more extendable and retractable load-bearing feet 225 of the walking aid 200 has a pivot at a connection to the linear rod 205 and one or more additional pivots along a length of the at least one of the two or more extendable and retractable load-bearing feet 225. In an aspect, at least one of the two or more extendable and retractable load-bearing feet 225 of walking aid 200 is rotatable. In some embodiments, at least one of the two or more extendable and retractable load-bearing feet 225 of the walking aid 200 is configured to retract for storage on a side of the linear rod 205. In some embodiments, at least one of the two or more extendable and retractable load-bearing feet 225 of the walking aid 200 is configured to retract for storage into an interior portion of the linear rod 205.

Walking aid 200 includes at least one first sensor 230 including circuitry configured to detect a parameter of a walking surface in proximity to the second end 215 of linear rod 205. The parameter of the walking surface can include at least one of a surface type, a friction coefficient, a roughness, a slope, an edge, a height, a distance, or a bump. In an aspect, the at least one first sensor 230 of walking aid 200 comprises at least one of a camera, a proximity sensor, a pressure sensor, an accelerometer, a gyroscope, a tactile sensor, a tilt sensor, an inclinometer, or a texture sensor.

Walking aid 200 of system 1000 further includes a controller 235 including a microprocessor 240 and circuitry 245. Controller 235 is operably coupled to the two or more extendable and retractable load-bearing feet 225 of walking aid 200 and the at least one first sensor 230. Circuitry 245 of controller 235 includes input circuitry configured to receive the first sensor output including information regarding the detected parameter of the walking surface and actuation circuitry configured to actuate at least one of the two or more extendable and retractable load-bearing feet 225 in response to the information regarding the detected parameter of the walking surface. In some embodiments, the actuation circuitry of circuitry 245 is configured to actuate at least one of the two or more extendable and retractable load-bearing feet 225 in response to the information regarding the detected parameter of the walking surface before the at least one of the two or more extendable and retractable load-bearing feet 225 contacts the walking surface. In some embodiments, the actuation circuitry of circuitry 245 is configured to independently and automatically actuate the two or more extendable and retractable load-bearing feet 225 to a configuration based on a position of the walking aid 200 relative to the walking surface. In some embodiments, the actuation circuitry of circuitry 245 is configured to independently and automatically actuate the two or more extendable and retractable load-bearing feet 225 to a configuration based on a position variance of the walking aid 200 from vertical.

Walking aid 200 further includes at least one second sensor 250 including circuitry configured to detect a parameter of the walking aid 200 and to transform the detected parameter of the walking aid 200 into a second sensor output. The parameter of the walking aid can include at least one of movement, speed, location, tip motion of the walking aid, position of the walking aid relative to the walking surface, or tilt angle of the walking aid relative to the walking surface. In an aspect, the at least one second sensor 250 of walking aid 200 includes at least one of a camera, an accelerometer, a gyroscope, a GPS receiver, or a clock. In some embodiments, the at least one second sensor 250 includes at least one of a tilt sensor, an inclinometer, a pressure sensor, or a proximity sensor. In some embodiments, the controller 235 is operably coupled to the at least one second sensor 250, the input circuitry configured to receive the second sensor output including information regarding the detect parameter of the walking aid and the actuation circuitry configured to actuate at least one of the two or more extendable and retractable load-bearing feet 225 in response to the information regarding the detected parameter of the walking aid 200. In some embodiments, the actuation circuitry of controller 235 is configured to actuate the at least one of the two or more extendable and retractable load-bearing feet 225 in response to movement of the walking aid 200 after it loses contact with the walking surface.

Walking aid 200 further includes transmission unit 255 operably coupled to the at least one second sensor 250, the transmission unit 255 including an antenna 260 and circuitry configured to transmit the second sensor output to computing device 1010. In an aspect, the transmission unit 255 of walking aid 200 comprises an optical transmission unit. In an aspect, the transmission unit 225 of walking aid 200 comprises a radiofrequency transmission unit.

Additional non-limiting aspects of a walking aid including a linear rod, a hand grip, two or more extendable and retractable load-bearing feet, at least one first sensor, a controller, at least one second sensor, and a transmission unit have been described above herein.

System 1000 further includes computing device 1010 including processor 1020 and circuitry 1030. Circuitry 1030 includes input circuitry 1040 configured to receive the second sensor output including information regarding the detected parameter of the walking aid 200 and calculation circuitry 1050 configured to calculate a gait value based on the received information regarding the detected parameter of the walking aid 200.

In an aspect, computing device 1010 comprises a dedicated handheld device. For example, the computing device can include a dedicated handheld device manufactured specifically for use with the walking aid. Said dedicated handheld device can further include a receiver, a microprocessor, and circuitry including input and calculation circuitry. In an aspect, computing device 1010 comprises a mobile communication device. For example, the computing device can include a smart phone, cell phone, or similar device that includes a receiver capable of communicating with the walking aid and circuitry configured to receive and process information from the walking aid to calculate a gait value for a user. In an aspect, the computing device 1010 comprises a tablet computer, a laptop computer, or a desktop computer. In some embodiments, the computing device 1010 is connected to a network. For example, the computing device can be connected to or in communication with a network associated with a caregiver and/or healthcare provider. In an aspect, the computing device 1010 is part of a cloud computing system.

In an aspect, the computing device 1010 includes, but is not limited to, mobile computing devices (e.g., hand-held portable computers, Personal Digital Assistants (PDAs), laptop computers, netbook computers, tablet computers, and so forth), mobile telephone devices (e.g., cellular telephones and smartphones), devices that include functionalities associated with smartphones and tablet computers (e.g., phablets), portable game devices, portable media layers, multimedia devices, satellite navigation devices (e.g., Global Positioning System (GPS) navigation devices), e-book reader devices (eReaders), Smart Television (TV) devices, surface computing devices (e.g., table top computers), Personal Computer (PC) devices, and other devices that employ touch-based human interfaces.

Computing device 1010 includes a processor 1020. The computing device further includes a system memory and a system bus that couples various system components including the system memory to the processor. The processor 1020 can include a microprocessor, a processing unit, a central processing unit (CPU), a digital signal processor (DSP), an application-specific integrated circuit (ASIC), a field programmable gate entry (FPGA), or the like, or any combinations thereof, and can include discrete digital or analog circuit elements or electronics, or combinations thereof. In an aspect, the computing device includes one or more ASICs having a plurality of pre-defined logic components. In an aspect, the computing device includes one or more FPGA having a plurality of programmable logic commands.

In an aspect, the computing device 1010 is operably coupled to one or more input/output components, e.g., one or more user interface components. In an aspect, the one or more input/output components are connected to the processor of the computing device through one or more user input interfaces that are coupled to the system bus, but may be connected by other interfaces and bus structures, such as a parallel port, game port, or a universal serial bus (USB). For example, external input components or output components may be connected to the processor through a USB port. The computing device may further include or be capable of connecting to a flash card memory. The computing device may further include or be capable of connecting with a network through a network port and network interface, and through wireless port and corresponding wireless interface may be provided to facilitate communication with other peripheral devices, for example, the walking aide, a smart phone, a computer, a display monitor, and/or a printer.

In an aspect, image-based applications such as viewers and/or toolkits (e.g., Insight Segmentation and Registration Toolkit (ITK)), are incorporated for further intake of information. In an aspect, CAD implementations, image segmentation, or other image analysis algorithms may allow processing of images received from one or more sensors (e.g., cameras) associated with the walking aid.

Computing device 1010 includes a memory component. The memory component can include memory chips, e.g., ROM or flash memory chips, for providing storage of operating systems, look-up tables, references datasets, and algorithms for comparing input data or information with reference data or information. The memory component of the computing device may include read-only memory (ROM) and random access memory (RAM). A number of program modules may be stored in the ROM or RAM, including an operating system, one or more application programs, other program modules and program data.

Computing device 1010 includes computer-readable media products and may include any media that can be accessed by the computing device including both volatile and nonvolatile media, removable and non-removable media. By way of example, and not of limitation, computer-readable media may include non-transitory signal-bearing media. Non-limiting examples of non-transitory signal-bearing media include a recordable type medium such as magnetic tape, a hard disk drive, digital tape, computer memory, or the like, as well as transmission type medium such as a digital and/or analog communication medium (e.g., fiber optic cable, waveguide, wired communications link, wireless communication link). Further non-limiting examples of signal-bearing media include, but are not limited to, flash memory, magnetic tape, MINIDISC, non-volatile memory card, EEPROM, optical disk, optical storage, RAM, ROM, system memory, web server, cloud, or the like. By way of example, and not of limitation, computer-readable media may include computer storage media, e.g., magnetic tape, magnetic disk storage, optical disk storage, memory cards, flash memory cards, electrically erasable programmable read-only memory (EEPROM), solid state RAM, and solid state ROM or any other medium which can be used to store the desired information and which can be accessed by the computing device. By way of further example, and not of limitation, computer-readable media may include a communication media, e.g., wired media, such as a wired network and a direct-wired connection, and wireless media such as acoustic, RF, optical, and infrared media.

Computing device 1010 includes input circuitry 1040 configured to receive the second sensor output including information regarding the detected parameter of the walking aid. The second sensor output received by the computing device from one or more accelerometers, gyroscopes, cameras, a GPS receiver, tilt sensor, inclinometers, and clocks associated with the walking aid includes information regarding a detected parameter of the walking aid. The detected parameter of the walking aid can include at least one of movement, speed, location, tip motion of the walking aid, position of the walking aid relative to the walking surface, or tilt angle of the walking aid relative to the walking surface. In an aspect, the second sensor output received by the input circuitry 1040 provides an indication of how often a user is using the walking aid and for how long, as indicated by data from accelerometers, motion detectors, and clocks associated with the walking aid. In an aspect, the second sensor output received by the input circuitry 1040 provides an indication of where a user is going and how far with the walking aid, as indicated by data from a GPS receiver associated with the walking aid or through access to a local positioning system. In an aspect, the second sensor output received by the input circuitry 1040 provides an indication as to a path (e.g., a straight versus weaving path) a user is taking with the walking aid. In an aspect, the second sensor output received by the input circuitry 1040 provides an indication as to how fast a user is traveling with the walking aid, as indicated by data from a speedometer sensor. In an aspect, the second sensor output received by the input circuitry 1040 provides an indication of the quality of the walking, e.g., is the user weaving, shuffling, leaning to one side, or other, as indicated from data from accelerometers, gyroscopes, tilt sensors, and/or inclinometers associated with the walking aid. In an aspect, the second sensor output received by the input circuitry 1040 includes one or more images captured with a digital camera associated with the walking aid, the one or more images providing information regarding a gait of a user.

Computing device 1010 includes calculation circuitry 1050 configured to calculate a gait value based on the received information regarding the detected parameter of the walking aid 200. In an aspect, the calculation circuitry includes one or more algorithms configured to calculate the gait value based on the received information regarding the detected parameter of the walking aid. The one or more algorithms can include machine learning algorithms, non-limiting examples of which include linear regression, logistic regression, decision tree, SVM (Support Vector Machine), Naïve Bayes, KNN (K-nearest neighbors), Dimensionality Reduction Algorithms, artificial neural networks, and the like. In an aspect, the calculation circuitry includes non-transitory computer readable medium including computer readable instructions for calculating a gait value based on the received information regarding the detected parameter of the walking aid. In an aspect, the calculation circuitry includes modeling capabilities configured to generate a motion model of the walking aid relative to the user and the walking surface over time and to calculate the gait value based on the motion model. In an aspect, the calculation circuitry is configured to access look-up tables, feature maps, training sets, and/or databases including reference data points and/or motion models for comparison with the received information regarding the detected parameter of the walking aid.

Walking requires coordination of various nerve and muscle functions to develop and/or maintain a normal gait. A normal gait requires an intact and functioning peripheral nervous system to send messages from the muscle and joints to the brain and receive messages back. Good balance is generally a central nervous system function, but functioning bone and joint systems are also required for a normal gait—any problems in these systems will cause ambulation difficulties. For example, if a muscle is weak or a joint is painful, the gait slows down. For example, if one leg is weak or painful, the gait or stride may be uneven or off balance. Normal walking gait includes three specific phases: the stance phase (single leg support or one foot on the ground—40% of the total cycle); the double stance phase (both feet on the ground—20% of the total cycle); and the swing phase (leg is suspended in mid-air—40% of the total cycle). Stride length is the distance from the big toe leaving the ground (toe off) and heel hitting the ground (heel plant).

The calculated gait value can be an overall assessment of the user's gait based on the information regarding the detected parameter of the walking aid, i.e., the movement or lack thereof of the walking aid relative to the user and/or the walking surface. The calculation of the gait value can take into consideration the pace, rhythm, intra-step variability, and asymmetry of the user's gait. In an aspect, the gait value can take into consideration one or more spatiotemporal subcomponents of gait including at least one of velocity (distance traversed divided by time), cadence (steps per minute; stepping frequency), double support time (double stance phase; period within a stride during which both feet are simultaneously on the ground), single limb support time (stance phase; time elapsed with only one foot in contact with the ground), stride length (distance in anterior-posterior direction between successive point of contact of the same foot (i.e., two steps)), step length (distance in anterior-posterior direction between successive points of contact of opposite foot (i.e., one step)), stride time (time elapsed between successive points of contact of the same foot (i.e., two steps)), step time (time elapsed between successive points of contact of opposite foot (i.e., one step)), step width (distance in medio-lateral direction between two feet (i.e., base of support)), swing time (swing phase; time elapsed with one foot in the air and one on the ground), variability (calculated coefficient of variation or standard deviation of one of the above, e.g., step length, stride length, step time, stride time, and/or step width; and asymmetry (differences between symmetrical spatiotemporal characteristics of left and right leg during gait)).

In an aspect, the calculated gait value provides an indication of the posture, balance, and/or mobility of the user. For example, if the user is only periodically using the walking aid, this may indicate a change in mobility. In some instances, the change in mobility might be improved mobility of the user such that the walking aid is no longer needed, thus accounting for the reduced frequency of use. In some instances, the change in mobility might indicate reduced mobility, either through a physical condition (e.g., increased joint, muscle, and/or nerve pain, or worsening heart and/or lung disease) or a mental condition (e.g., worsening depression or dementia and decreased desire to move about). For example, if the user is dropping the walking aid more frequently, this may indicate a change in grip strength or balance and a possible increased risk of falling. For example, if the at least one second sensors indicate that the walking aid is being held at an awkward angle, this may indicate a change in posture.

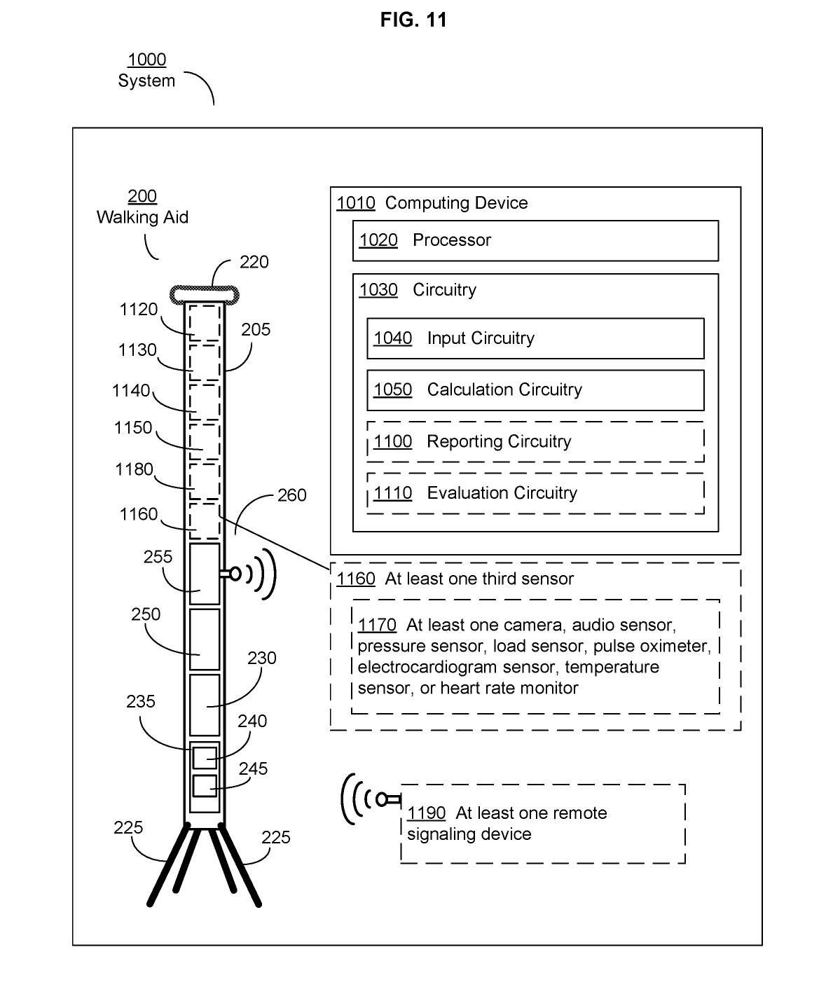

FIG. 11 shows additional aspects of system 1000. In an aspect, computing device 1010 of system 1000 further includes reporting circuitry 1100. In some embodiments, the computing device includes reporting circuitry configured to report the calculated gait value to at least one of a digital display, a reporting device, or a second computing device. In some embodiments reporting circuitry 1100 is configured to report the calculated gait value to a digital display. For example, the reporting circuitry can be configured to report the calculated gait value to a digital display associated with the computing device, the calculated gait value represented as text, numbers, images, graphs, plots, and/or tables.

In some embodiments, reporting circuitry 1100 is configured to report the calculated gait value to a reporting device. The reporting device can include at least one of a digital display, an optical reporting device, an audio reporting device, or a haptic reporting device. In some embodiments, the reporting device is associated with the computing device. For example, the reporting device can include an audio function associated with a smart phone that is able to provide an audible report to a user with information regarding the calculated gait. In some embodiments, the reporting device is associated with the walking aid. For example, the reporting device can include a digital display or optical, audio, or haptic reporting device associated with the walking aid and be directly or indirectly in communication with the computing device. For example, the reporting device can be part of a warning system associated with the walking aid. For example, the reporting device can be part of a user interface associated with the walking aid. In an aspect, the reporting device is configured to provide feedback to the user regarding the calculated gait value. For example, the reporting device can provide feedback indicating that the user's calculated gait value falls within a range of acceptable gait values. For example, the reporting device can provide feedback indicating whether the user's calculated gait value fails to fall within a range of acceptable gait values. The acceptable gait value can include a normalized gait value matched to the user's age, gender, height, weight, and medical/mental condition. The acceptable gait value can include a calibrated gait value generated by performing a specific set of walking movements. (See, e.g., U.S. Patent Publication 2016/0262661 from Sarkar et al., which is incorporated herein by reference.)

In some embodiments, reporting circuitry 1100 is configured to report the calculated gait value to a second reporting device. For example, the reporting circuitry associated with a mobile communication device (e.g., a smart phone) can be configured to report the calculated gait value to a second reporting device associated with a caregiver or medical provider. For example, the reporting circuitry can be configured to provide feedback regarding a user's calculated gait value to a cell phone, computing device, or network associated with a caregiver and/or medical provider.

In some embodiments, the computing device further includes reporting circuitry configured to report the calculated gait value to a network. For example, the reporting circuitry can be configured to report the calculated gait value to a network associated with a caregiver and/or health provider. For example, the reporting circuitry can be configured to report the calculated gait value to an electronic medical record. For example, the reporting circuitry can be configured to report the calculated gait value to a cloud computing system.

In some embodiments, reporting circuitry 1100 is configured to provide feedback directly to the user regarding the calculated gait value. For example, the reporting circuitry can be configured to transmit a signal to an audio, haptic, or optical reporting device associated with the walking aid to provide audio, haptic, or optical feedback regarding the calculated gait value. In an aspect, the reporting circuitry is configured to provide feedback to a user once a threshold gait value has been achieved. In some embodiments, the threshold gait value is a positive value, e.g., a normalized or calibrated gait value to which a user is trying to maintain or achieve. In some embodiments, the threshold gait value is a negative value, e.g., a worsening of a gait value. In an aspect, the reporting circuitry is configured to alert a user if he/she varies from a normalized or calibrated gate value. For example, the hand grip of the walking aid can vibrate or provide an audible beep if the user varies from a normalized or calibrated gate value. In some embodiments, the reporting circuitry provides feedback to a remote device, e.g., a second computing device or a network.

In some embodiments, circuitry 1030 of computing device 1010 further includes evaluation circuitry 1110 configured to evaluate a condition of a user based on a comparison of the calculated gait value with at least one reference gait value; and reporting circuitry 1100 configured to report the evaluated condition to the user. In an aspect, the computing device 1010 can include a database of stored reference gait values correlated with medical or mental conditions. In an aspect, the at least one reference gait value is one or more previously calculated gait values for the user. In an aspect, the at least one reference gait value is derived from one or more matched individuals. For example, the reference gait value may be matched to a user based on age, weight, gender, height. For example, the reference gate value may be matched to a user based on a medical or mental condition. In an aspect, the calculated gait value when compared with the at least one reference gait value indicates a new or worsening medical and/or mental condition, e.g., heart disease, spinal cord compression associated myelopathy (loss of balance, grip, and increased clumsiness), chronic obstructive pulmonary disease, arthritis, osteoporosis, dementia, stroke, cognitive decline, depression, multiple sclerosis, and the like. Changes in gait may be indicative of arthritic joints or peripheral neuropathies. A spastic gait may be indicative of vascular injuries to the brain (strokes) or tumors. A vestibular gait may be indicative of a dysfunction in the inner ear. In an aspect, the calculated gait value as it relates to gait velocity is indicative of a neuropathology within the higher cortical regions and may be linked to at least one of vasodilation reserve, age-related white matter disease, brain ventricle volume, loss of white matter, dysfunction of the cholinergic neurotransmitter system, atrophies in sensorimotor and frontoparietal regions, and inflammatory damage and reduced volume of the primary motor cortex. The calculated gait value may be further associated with a decline in cognitive function, attention, onset to mild cognitive impairment, dementia, and predicted time to morbidity. See, e.g., Kraan et al. (2017) “The development dynamics of gait maturation with a focus on spatiotemporal measures,” Gait & Posture 51:208-217, which is incorporated herein by reference.

In an aspect, the calculated gait value when compared with the at least one reference gait value indicates improvement or lack thereof as a result of an exercise and/or physical therapy/occupational therapy program. For example, the system including the walking aid and computing device may be used to monitor improvements and/or progression of gait in response to medication, psychotherapy, physical therapy, and/or exercise. For example, the system including the walking aid and computing device may be used to monitor improvements and/or progression of gait in response to alternative medicine therapies, e.g., massage, acupuncture, naturopathic medicines, chiropractic therapy, and the like.

In an aspect, the reporting circuitry 1100 of computing device 1010 is configured to report the evaluated condition of the user to at least one of a haptic reporting device, an audio reporting device, an optical reporting device, or a digital display. In an aspect, the at least one haptic reporting device, audio reporting device, optical reporting device, or digital display is associated with the walking aid 200. In an aspect, the at least one haptic reporting device, audio reporting device, optical reporting device, or digital display is associated with the computing device 1010. In an aspect, the reporting circuitry 1100 of the computing device 1010 is configured to report the evaluated condition of the subject to a second computing device. For example, the system can include a smart phone in communication with the walking aid and including reporting circuitry configured to report the evaluated condition of the subject to a second computing device (e.g., a tablet, laptop, or desktop computer) associated with a care provider or medical practitioner. In an aspect, the reporting circuitry 1100 of the computing device 1010 is configured to report the evaluated condition of the subject to a cloud computing system. In an aspect, the reporting circuitry 1100 of the computing device 1010 is configured to report the evaluated condition of the user to a network. For example, the system can include reporting circuitry configured to report the evaluated condition of the user to a hospital or clinic network. For example, the system can include reporting circuitry configured to report the evaluated condition of the user to the user's electronic medical record.

With reference to FIG. 11, in some embodiments, the walking aid 200 includes a user-activated control mechanism 1120 including circuitry configured to control extension and retraction of the two or more extendable and retractable load-bearing feet 225. In some embodiments, the walking aid 200 includes a mechanical energy harvester 1130 configured to store electrical or mechanical energy to power actuation of the two or more extendable and retractable load-bearing feet 225. In some embodiments, the walking aid 200 includes a warning system 1140 operably coupled to the controller 235 and configured to alert a user, wherein the warning system comprises at least one of an optical warning system, an audible warning system, or a haptic warning system. In some embodiments, the walking aid 200 of system 1000 includes a user interface 1150. Additional non-limiting aspects of a walking aid including a user-activated control mechanism, a mechanical energy harvester, a warning system, and/or a user interface have been described above herein.

In some embodiments, the walking aid 200 includes at least one third sensor 1160 operably coupled to the transmission unit 255 and including circuitry configured to detect a physiological parameter of a user and to transform the detected physiological parameter of the user into a third sensor output, wherein the transmission unit 255 includes circuitry configured to transmit the third sensor output to the computing device 1010, wherein the computing device 1010 includes input circuitry 1040 configured to receive the third sensor output including the information regarding the detected physiological parameter of the user and reporting circuitry 1100 configured to report the detected physiological parameter of the user. In an aspect, the at least one third sensor 1160 includes at least one camera, audio sensor, pressure sensor, load sensor, pulse oximeter, electrocardiogram sensor, temperature sensor, or heart rate monitor, as illustrated in block 1170. Non-limiting aspects of sensors for detecting a physiological parameter of the user have been described herein.

In some embodiments, the calculation circuitry includes the third sensor output in the calculation of the gait value. For example, the calculation circuitry can take into account weight shifts as measured by changes in pressure or load applied to the walking aid in calculating the gait value. For example, the calculation circuitry can take into account shuffling or foot-strike sounds as measured by an audio sensor in calculating the gait value. For example, the calculation circuitry can take account visual images of the user walking with the walking aid as measured using a camera or other image capture device to calculate the gait value. In some embodiments, the calculation circuitry includes the first sensor output, the second sensor output, and the third sensor output in the calculation of the gait value. For example, the calculation circuitry can take into account the information regarding the detected parameter of the walking surface, the detected parameter of the walking aid, and the detected physiological parameter of the user to calculate the gait value.

In some embodiments, the evaluation circuitry includes the third sensor output in evaluating a medical or mental condition of a user. For example, speech patterns measured with an audio sensor might be combined with a calculated gait value to assess a mental condition, e.g., a cognitive condition, of a user. For example, heart rate and breathing sounds as measured with an audio sensor might be combined with a calculated gait value to assess a medical condition, e.g., progression of heart failure, of a user.

In some embodiments, the walking aid 200 further includes at least one receiver 1180 including circuitry configured to receive a signal from the computing device 1010. For example, the walking aid can include a receiver for receiving transmitted signals from at least one of a mobile communication device, a dedicated hand-held device, a computer (e.g., a laptop, tablet, or desktop computer), a network, a server, or other device or system. In some embodiments, the at least one receiver 1180 includes circuitry configured to receive a transmitted signal from a wearable device, e.g., a fitness monitor, heart rate monitor, pedometer, inertial sensor, electromyography sensor, or similar type of device worn on the user. The information received by the receiver from the wearable device can be added to or correlated with information incorporated into the first and second sensor outputs. The information received can be added to or correlated with information incorporated in a third sensor output including a detected physiological parameter of the user. The at least one receiver 1180 can include an optical receiver for receiving optical signal transmission. The at least one receiver 1180 can include a radiofrequency receiver for receiving radiofrequency signal transmission. The receiver can include at least one of an electromagnetic signal receiver, a magnetic signal receiver, a microwave signal receiver, an acoustic signal receiver, an optical signal receiver, or other receiver types and configured to receive at least one of a radio signal, a magnetic signal, an acoustic signal, an electromagnetic signal, or an optical signal. In an aspect, the at least one receiver 1180 includes an antenna configured to intercept signals from computing device 1010 or from one or more other external devices. In an aspect, at least one receiver 1180 includes at least one filter, e.g., an electronic filter, a frequency filter, or a bandwidth filter, to separate specific signals from the computing device from other signals in the environment. In an aspect, the at least one receiver 1180 includes a digital signal processor. In an aspect, the at least one receiver 1180 and transmitter 255 are part of a transceiver or similar type of component capable of both transmitting and receiving signals.

In some embodiments, the at least one receiver 1180 of walking aid 200 includes an antenna configured to intercept signals emitted from other devices. In some embodiments, the other devices include wearable devices worn by the user, e.g., fitness monitors, heart rate monitors, pulse oximeters, or other wearable devices. In some embodiments, the other devices include at least one remote signaling device. In an aspect, system 1000 further includes at least one remote signaling device 1190 in wireless communication with at least one receiver 1180 associated with the walking aid 200, the at least one receiver 1180 including circuitry configured to receive a signal from the at least one remote signaling device 1190 and to transform the received signal into information regarding a location of the walking aid 200 in an environment. In an aspect, the at least one remote signaling device 1190 includes one or more beacons configured to emit a radio signal, a magnetic signal, an acoustic signal, an electromagnetic signal, or an optical signal. In an aspect, the at least one remote signaling device 1190 includes a local positioning system.

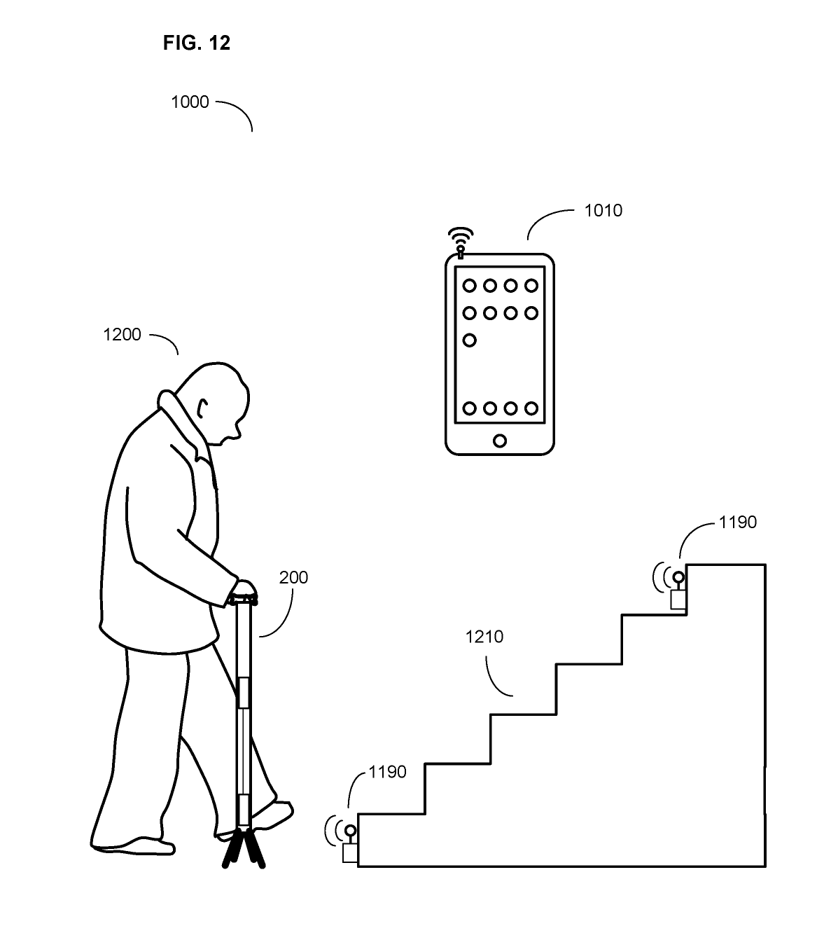

With reference to FIG. 12, shown is an embodiment of system 1000 including a walking aid, a computing device, and a remote signaling device. FIG. 12 shows system 1000 including walking aid 200, computing device 1010 and remote signaling device 1190. Walking aid 200 includes at least one receiver operably coupled to the controller. Remote signaling device 1190 is in wireless communication with the at least one receiver of walking aid 200 and is configured to transmit a signal. The receiver of walking aid 200 is configured to receive the signal from the remote signaling device 1190 as a user 1200 approaches an environment 1210 (in this non-limiting example, a stepped walking surface) and configured to transform the signal into information regarding a location of the walking aid 200 in the environment 1210. For example, the remote signaling system may be situated such that it sends one or more signals from the top and/or bottom of a staircase to inform the walking aid that the user is approaching or is in the process of engaging a staircase. The controller includes actuation circuitry configured to actuate at least one of the two or more extendable and retractable load-bearing feet based on the information regarding the location of the walking aid 200 in the environment 1210.

In an aspect, the at least one receiver of walking aid 200 comprises at least one electromagnetic signal receiver configured to receive an electromagnetic signal (e.g., ultraviolet, visible light, infrared, microwave, a radio wave signals) from a remote electromagnetic signal generator and configured to transform the electromagnetic signal into information regarding a location of the walking aid in an environment.

In an aspect, the at least one receiver of walking aid 200 comprises at least one magnetic signal receiver including circuitry configured to receive a magnetic signal from a remote magnetic field signal generator and configured to transform magnetic signal into information regarding a location of the walking aid in an environment. In an aspect, the at least one receiver includes a magnetometer configured to measure a strength and direction of magnetic field at a point in space. In an aspect, the at least one receiver includes a MEMS magnetometer. Other non-limiting examples of receivers of detecting a magnetic field include a magneto-diode, magneto-transistor, anisotropic magnetoresistance (AMR) magnetometer, giant magnetoresistance (GMR) magnetometer, magnetic tunnel junction magnetometer, magneto-optical sensor, Lorentz-force-based MEMS sensor, fluxgate magnetometer, or search coil magnetic field sensor.

In an aspect, the at least one receiver of walking aid 200 comprises at least one radiofrequency signal receiver including circuitry configured to receive a radiofrequency signal from a remote radiofrequency signal generator and configured to transform the radiofrequency signal into information regarding a location of the walking aid in an environment. In an aspect, the at least one radiofrequency signal receiver is tuned to receive a specific frequency. In an aspect, the at least one radiofrequency signal receiver includes a radiofrequency module, e.g., a superheterodyne receiver or a super-regenerative receiver. In an aspect, the at least one radiofrequency module is part of an electronic circuit and can include a WLAN module, a Zigbee module, a Bluetooth module, a GPS module, an RFID module, or a UHF module (from, e.g., Future Electronics, Pointe Claire, Quebec, Canada).

In an aspect, the at least one receiver of walking aid 200 comprises at least one photodetector configured to receive an optical signal from a remote optical signal generator and configured to transform the optical signal into information regarding a location of the walking aid in an environment. For example, the walking aid can include one or more photodetectors configured to detect a specific wavelength of light or other electromagnetic energy emitted by the remote signaling device. In an aspect, the at least one photodetector is incorporated into a camera. Non-limiting examples of photodetectors include photodiodes, metal-semiconductor-metal (MSM) photodetectors, phototransistors, photoresistors, photomultipliers, pyroelectric photodetectors, and thermal detectors. In an aspect, the at least one receiver comprises an array of photodetectors. Photodetectors are available from commercial sources (from, e.g., AC Photonics, Santa Clara, Calif.; Electro-Optics Technology, Inc., Traverse City, Mich.; and OSI Laser Diode, Inc., Edison, N.J.).

In an aspect, the at least one receiver of walking aid 200 comprises at least one infrared sensor including circuitry configured to receive an infrared signal from a remote infrared signal generator and configured to transform the infrared signal into information regarding a location of the walking aid in an environment. For example, the walking aid can include components of an infrared sensitive charge-coupled device (CCD) camera. For example, the walking aid can include at least one passive infrared sensor configured to detect infrared light emitted by the remote signaling device. A non-limiting example of a passive infrared sensor includes a solid state sensor made from one or more pyroelectric materials, e.g., lithium tantalite and/or strontium barium niobate. Infrared detectors are available from commercial sources (from, e.g., SparkFun Electronics, Niwot, Colo.).

In an aspect, the at least one receiver of walking aid 200 comprises at least one microphone configured to receive an audio/acoustic signal from a remote audio/acoustic signal generator and configured to transform the audio/acoustic signal into information regarding a location of the walking aid in an environment. For example, the walking aid can include at least one microphone configured to detect audible sound, e.g., audible beeping, emitted from the remote signaling device. In some embodiments, the at least one microphone is configured to detect infrasound or low-frequency sound that is at a frequency below the “normal” limit of human hearing or below about 20 hertz or cycles per second. In some embodiments, the at least one microphone includes a transducer configured to detect ultrasound. In an aspect, the at least one microphone includes a sound transducer, e.g., a dynamic moving coil, electret condenser, ribbon, or piezo-electric crystal sound transducer.

Returning to FIG. 12, in some embodiments, system 1000 includes at least one remote signaling device 1190 including circuitry configured to transmit a signal. For example, the remote signaling device can include a transmitter which transmits a continuous or periodic signal. In an aspect, system 1000 includes one remote signaling device 1190. For example, a single remote signaling device can be placed in association with a walking hazard, e.g., an uneven patch of walking surface, to communicate with a walking aid when it comes into range or proximity to the remote signaling device and the associated walking hazard. In an aspect, system 1000 includes two or more remote signaling devices 1230. For example, two or more remote signaling devices can be placed along a walking surface, e.g., a staircase or stairwell, to communicate with a walking aid when it comes into range or proximity of the two or more remote signaling devices.

In an aspect, the remote signaling device 1190 includes one or more beacons. In some embodiments, the one or more beacons include one or more radio beacons configured to emit a radio frequency, e.g., high frequency (HF), very high frequency (VHF), ultra-high frequency (UHF), or microwave frequencies. In an aspect, at least one of the one or more beacons emits a radio signal, a magnetic field, an acoustic signal, an electromagnetic signal, or an optical signal. In an aspect, at least one of the one or more beacons includes a camera. In an aspect, at least one of the one or more beacons includes Wi-Fi, inaudible sound waves, or LED lighting.

In an aspect, remote signaling device 1190 includes an electromagnetic signal generator configured to emit at least one of an ultraviolet signal, a visible light signal, an infrared signal, a microwave signal, or a radio wave signal. For example, the electromagnetic signal generator can include one or more radio or microwave beacons. In an aspect, the electromagnetic signal generator emits signals of a single or narrow bandwidth for selective detection by the at least one receiver on the walking aid. For example, the electromagnetic signal generator can include one or more infrared beacons configured to continuously or periodically transmit a modulated light beam in the infrared spectrum (e.g., 980 nanometers) and the walking aid can include a receiver with one or more filters to detect the specific wavelength of infrared energy. In an aspect, the electromagnetic signal generator includes an optical output. For example, the electromagnetic signal generator can include a laser or light emitting diode (LED) for emitting one or more beams of visible light. For example, the electromagnetic signal generator can include an infrared LED for emitting an infrared signal.

In an aspect, the remote signaling device 1190 includes a radiofrequency signal generator, e.g., one or more radio beacons configured to transmit radio signals detectable by a radiofrequency signal receiver associated with a walking aid. For example, the system can include one or more beacons configured to emit a radiofrequency, e.g., high frequency (HF), very high frequency (VHF), or ultra-high frequency (UHF). In an aspect, the radiofrequency signal generator generates a single or narrow radio frequency for detection by the at least one receiver on the walking aid. Non-limiting examples of systems making use of the radio spectrum include FM radios, radars, cellular networks, DECT phones, WLAN, ZigBee, RFID, ultra-wideband, high sensitive GNSS and pseudolite systems.

In an aspect, the remote signaling device 1190 includes a microwave signal generator, e.g., one or more microwave beacons configured to transmit microwave signals detectable by a microwave signal receiver associated with the walking aid. For example, the system can include one or more beacons configured to emit microwave frequencies (3-30 GHz). In an aspect, the microwave signal generator generates a single or narrow microwave frequency for detection by the at least one receiver on the walking aid.

In an aspect, remote signaling device 1190 includes a magnetic signal generator, e.g., a magnetic beacon including a current carrying coil, a capacitor, and a signal generator while the receiver associated with the walking aid includes a tri-axial search-coil magnetometer. See, e.g., Sheinker et al. (2013) “Localization in 3-D Using Beacons of Low Frequency Magnetic Field” IEEE Transactions on Instrumentation and Measurement, 62:3194-3201, which is incorporated herein by reference.

In an aspect, the remote signaling device 1190 includes an acoustic signal generator configured to emit at least one of sound, ultrasound, or infrasound. In an aspect, the acoustic signal generator emits an audible signal. In an aspect, the acoustic signal generator emits an acoustic signal just outside the range of human hearing. For example, the acoustic signal generator can emit infrasound or low-frequency sound that is at a frequency below the “normal” limit of human hearing or below about 20 hertz or cycles per second. In an aspect, the acoustic signal generator includes an audio sound transducer for generating acoustic waves.

In an aspect, the remote signaling device 1190 includes an RFID signal/receiver, e.g., a radiofrequency identification (RFID) tag readable by the at least one receiver on the walking aid. In an aspect, the RFID tag is powered and the at least one receiver includes an antenna of sufficient size and sensitivity to detect the signal from the tag. In an aspect, the remote signaling device includes an RFID tag capable of being read from a distance, e.g., 1-10 meters. For example, the walking aid can include an RFID reader capable of reading RFID tags at 5 meters. In an aspect, the read distance is dependent upon the RFID reader and antenna associated with the walking aid and the properties of the RFID tag. RFID tags and readers are available from commercial sources (from, e.g., SkyRFID, Inc., Toronto, Ontario, Canada). In an aspect, the RFID signal/receiver includes a passive RFID system. See, e.g., Sabesan et al. (2014) “Wide area passive UHF RFID system using antenna diversity combined with phase and frequency hopping” IEEE Transactions on Antennas and Propagation, 62:878-888, which is incorporated herein by reference.

In an aspect, the remote signaling device 1190 includes a local positioning system including three or more signaling beacons distributed throughout a user's residence and particularly associated with hazardous or potentially hazardous walking surfaces, e.g., stairs or surface transitions. The signaling beacons can emit one or more of radio signals, electromagnetic signals, magnetic signals, acoustic signals, or optical signals. In an aspect, local positioning system includes a real-time locating system. In an aspect, local positioning system includes an indoor positioning system. In an aspect, local positioning system includes a hybrid positioning system. Non-limiting examples of technologies for use in a local position system include cameras, infrared, acoustic, WLAN/WiFi, RFID, ultra-wideband, high sensitive GNSS, pseudolites, inertial navigation, and magnetic systems.

In some embodiments, controller 235 of walking aid 200 includes circuitry configured to correlate the information regarding the location of the walking aid in the environment with a map of the environment. In an aspect, the map includes a map derived from a global positioning system (GPS). In an aspect, the map includes a map derived from a local positioning system. In an aspect, the map includes a floor plan of a house or other building. For example, the map can include a map of the user's home. For example, the map can include a map of a location frequented by the user, e.g., a school, a health clinic or hospital, an airport, a shopping mall, or other place of business. In an aspect, the map includes information regarding the walking surfaces associated with the map. For example, the map can include information regarding the surface of the walking surface, e.g., smooth, rough, stepped, or uneven, or transition points in the walking surface, e.g., transition from one material to another, changes in slope, or steps.

In an aspect, system 1000 further includes non-transitory machine readable media including one or more instructions implemented on the computing device 1010, the one or more instructions including, one or more instructions for receiving the second sensor output including information regarding the detected parameter of the walking aid; one or more instructions for calculating the gait value based on the received information regarding the detected parameter of the walking aid; one or more instructions for comparing the calculated gait value with at least one reference gait value; one or more instructions for evaluating a condition of the user based on the comparison between the calculated gait value and the at least one reference gait value; and one or more instructions for reporting the evaluated condition of the user. The non-transitory machine readable media including the one or more instructions can be implemented on at least one of a dedicated hand-held device, a mobile communication device, or a laptop, tablet, or desktop computer. The non-transitory machine readable media including the one or more instructions can be implemented through cloud computing.

Described herein are aspects of a walking aid. In an embodiment, a walking aid includes a linear rod having a first end and a second end; a hand grip disposed in proximity to the first end of the linear rod; two or more extendable and retractable load-bearing feet at the second end of the linear rod, at least one of the two or more extendable and retractable load-bearing feet including two or more telescoping segments; at least one first sensor including circuitry configured to detect a parameter of a walking surface in proximity to the second end of the linear rod and to transform the detected parameter of the walking surface into a first sensor output; at least one second sensor including circuitry configured to detect a parameter of the walking aid and to transform the detected parameter of the walking aid into a second sensor output; a reporting device; and a computing component including a microprocessor and circuitry, the computing component operably coupled to the two or more extendable and retractable load-bearing feet, the at least one first sensor, the at least one second sensor, and the reporting device, and including input circuitry configured to receive the first sensor output including information regarding the detected parameter of the walking surface and to receive the second sensor output including information regarding the detected parameter of the walking aide; actuation circuitry configured to actuate at least one of the two or more extendable and retractable load-bearing feet in response to the received information regarding the detected parameter of the walking surface; calculation circuitry configured to calculate a gait value for a user based on the received information regarding the detected parameter of the walking aide; and reporting circuitry configured to transmit a signal to the reporting device in response to the calculated gait value.

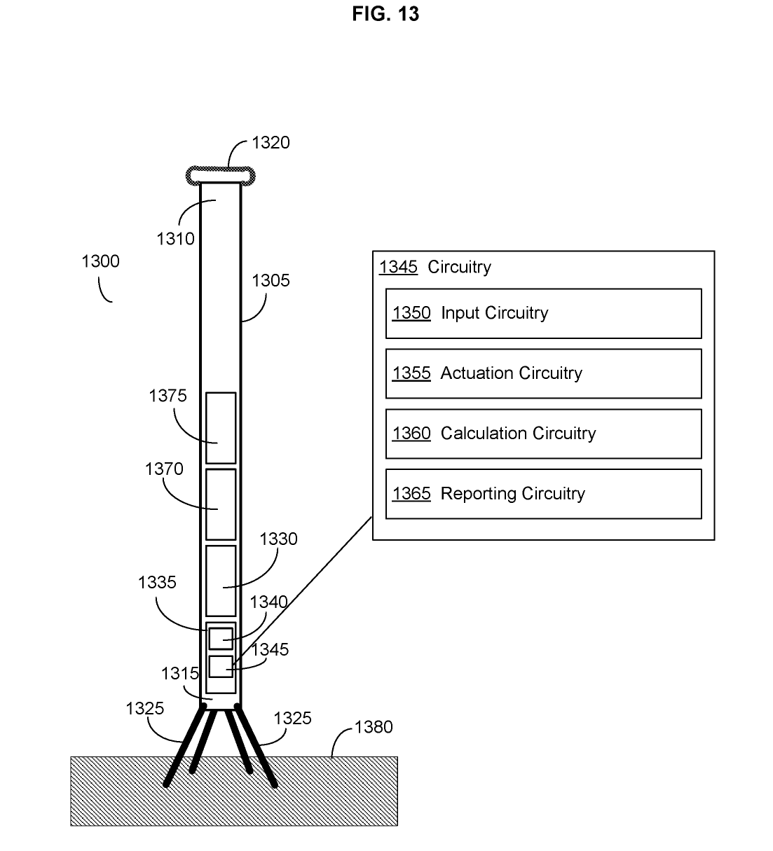

FIG. 13 illustrates further aspects of a walking aid. FIG. 13 shows a schematic of walking aid 1300 on walking surface 1380, e.g., a relatively flat walking surface. Walking aid 1300 includes a linear rod 1305 having a first end 1310 and a second end 1315 and hand grip 1320 disposed in proximity to the first end 1310 of linear rod 1305. Walking aid 1300 further includes two or more extendable and retractable load-bearing feet 1325 at the second end 1315 of linear rod 1305. Walking aid 1300 includes at least one first sensor 1330 including circuitry configured to detect a parameter of a walking surface 1380 in proximity to the second end 1315 of linear rod 1305 and to transform the detected parameter of the walking surface 1380 into a first sensor output. Walking aid 1300 further includes at least one second sensor 1370 including circuitry configured to detect a parameter of the walking aid 1300 and to transform the detected parameter of the walking aid 1300 into a second sensor output. Walking aid 1300 includes reporting device 1375. Walking aid 1300 includes a computing component 1335 including a microprocessor 1340 and circuitry 1345. Computing component 1335 is operably coupled to the two or more extendable and retractable load-bearing feet 1325, the at least one first sensor 1330, the at least one second sensor 1370, and the reporting device 1375. Circuitry 1345 of computing component 1335 includes input circuitry 1350 configured to receive the first sensor output including information regarding the detected parameter of the walking surface 1380 and to receive the second sensor output including information regarding the detected parameter of walking aid 1300. Circuitry 1345 of computing component 1335 includes actuation circuitry 1355 configured to actuate at least one of the two or more extendable and retractable load-bearing feet 1325 in response to the information regarding the detected parameter of the walking surface 1380. Circuitry 1345 of computing component 1335 includes calculation circuitry 1360 configured to calculate a gait value for a user based on the received information regarding the detected parameter of the walking aid 1300. Circuitry 1345 of computing component 1335 includes reporting circuitry 1365 configured to transmit a signal to the reporting device 1375 in response to the calculated gait value.

Walking aid 1300 includes linear rod 1305. The linear rod provides the main structural support or shaft for the walking aid. The first end of the linear rod serves as a connection point for a hand grip. The second end of the linear rod serves as a connection point for the two or more extendable and retractable load-bearing feet. In an aspect, the linear rod 1305 provides an attachment point for the at least one first sensor 1330, the at least one second sensor 1370, the computing component 1335, and the reporting device 1375. In some embodiments, the linear rod provides an attachment point for one or more additional components of the walking aid. For example, the linear rod can provide an attachment point for a user-activated control mechanism, a user interface, a transmission unit, a receiver, at least one third sensor, an actuator, a mechanical energy harvester, a power source, and/or a warning system. In an aspect, the linear rod comprises a cylindrical rod that is circular in transverse cross-section. Alternatively, the linear rod may have a non-circular transverse cross-section (e.g., oval, square, triangular, or polygonal in transverse cross-section). In an aspect, the diameter or width of the linear rod is about 0.5 inches to about 3.0 inches. A linear rod with a greater or lesser diameter or width is contemplated and may depend upon the composition and/or configuration of the linear rod. In an aspect, the linear rod is about 12 inches to about 50 inches in length. The length of the linear rod can be longer or shorter depending upon the overall desired length of the walking aid, e.g., the combined length of the hand grip, the linear rod, and the two or more extendable and retractable load-bearing feet. In an aspect, the length of the linear rod is adjustable. For example, the linear rod can include two or more telescoping segments with twistable locks to adjust and fix the length of the linear rod. Additional non-limiting aspects of the dimensions of a linear rod are described above herein.

In an aspect, linear rod 1305 is a solid linear rod. In an aspect, hand grip 1320, the two or more extendable and retractable load-bearing feet 1325, the at least one first sensor 1330, the at least one second sensor 1370, the computing component 1335, and the reporting device 1375 are attached to an outer surface of a solid linear rod. In an aspect, at least a portion of the linear rod is hollow. For example, at least a portion of the linear rod forms a hollow tube. In an aspect, at least a portion of the two or more extendable and retractable load-bearing feet 1325, the at least one first sensor 1330, the at least one second sensor 1370, the computing component 1335, and the reporting device 1375 are disposed within an interior portion of linear rod 1305.

Preferably linear rod 1305 of walking aid 1300 is formed from a lightweight but sturdy, weight-bearing material, non-limiting examples of which include metal or metal alloys (e.g., aluminum or aluminum alloy; stainless steel; titanium or titanium alloy); a plastic or polymer material; a carbon fiber or a carbon fiber composite (e.g., carbon fiber-reinforced polymer or plastic; carbon fiber-epoxy resin composite or a carbon fiber-polyester, -vinyl ester, or -nylon composite); a composite material (e.g., aramid, aluminum, ultra-high-molecular-weight polyethylene, or fiber glass); and/or wood (e.g., ash or beech wood).

Walking aid 1300 includes hand grip 1320 disposed in proximity to the first end 1310 of the linear rod 1305. The hand grip is configured for gripping by a hand or hands of a user. In an aspect, the hand grip is disposed at the extreme end of the linear rod (e.g., attached to the top of the linear rod). In an aspect, the hand grip is incorporated into the first end of the linear rod (e.g., a portion of the first end of the linear rod at least partially covered by a rubberized material). In an aspect, the hand grip is a curved continuation of the linear rod (e.g., a curved portion at the first end of the linear rod that forms a hand grip). In an aspect, the hand grip is disposed in a region proximal to the first end of the linear rod (e.g., attached to the side of the linear rod in a region proximal to the first end of the linear rod, extended out from the linear rod like a handle). In an aspect, the hand grip is smooth. In an aspect, the hand grip is contoured to accommodate the fingers of a gripping hand (e.g., having an ergonomically compatible grip or a contoured gel grip). In an aspect, the hand grip is formed from the same material used to form the linear rod. In an aspect, the hand grip is a contiguous part of the linear rod. In an aspect, the hand grip is formed separately and subsequently attached to the linear rod. In an aspect, the hand grip is formed from a different material from that used to form the linear rod. In an aspect, the hand grip is formed from a metal or metal alloy (e.g., aluminum, stainless steel, titanium, brass, or silver); plastic (e.g., polyvinylchloride); foam (e.g., polyurethane foam rubber or latex foam rubber); rubber; wood; or cork.

Walking aid 1300 includes two or more extendable and retractable load-bearing feet 1325. In an aspect, a walking aid includes two to ten extendable and retractable load-bearing feet. For example, a walking aid can include two, three, four, five, six, seven, eight, nine, or ten extendable and retractable load-bearing feet. In an aspect, each of the two or more extendable and retractable load-bearing feet is about 2 inches to about 24 inches in length. The length of each of the extendable and retractable load-bearing feet can be longer or shorter depending upon the overall desired length of the walking aid, e.g., the combined length of the hand grip, the linear rod, and the two or more extendable and retractable load-bearing feet. In an aspect, at least one of the two or more extendable and retractable load-bearing feet is circular in transverse cross-section. Alternatively, at least one of the two or more extendable and retractable load-bearing feet may include a non-circular transverse cross-section. For example, at least one of the two or more extendable and retractable load-bearing feet can be oval, square, triangular, or polygonal in transverse cross-section. In an aspect, the diameter or width of at least one of the two or more extendable and retractable load-bearing feet is about 0.2 inches to about 3.0 inches. An extendable and retractable load-bearing foot with a larger or smaller diameter or width is contemplated and may depend upon the composition and/or configuration of the extendable and retractable load-bearing foot.

Preferably, the two or more extendable and retractable load-bearing feet are formed from a lightweight but sturdy material, e.g., a material capable of load-bearing, non-limiting examples of which include metal or metal alloys (e.g., aluminum or an aluminum alloy; stainless steel; or titanium or a titanium alloy); a plastic or polymer material; a carbon fiber or a carbon fiber composite (e.g., carbon fiber-reinforced polymer or plastic; or carbon fiber-epoxy resin composite or a carbon fiber-polyester, -vinyl ester, or -nylon composite); a composite material (e.g., aramid, aluminum, ultra-high-molecular-weight polyethylene, or fiber glass); and/or wood (e.g., ash or beech wood).

Walking aid 1300 includes two or more extendable and retractable load-bearing feet 1325, at least one of which includes two or more telescoping segments. For example, an extendable and retractable load-bearing foot can include a first segment configured to slide into or out of an interior portion of an adjacent second segment to adjust the overall length of the foot. In an aspect, an extendable and retractable load-bearing foot can include three or more telescoping segments. In an aspect, at least one of the telescoping segments is a hollow tube of a load-bearing metal or metal alloy. For example, at least one of the telescoping segments can be a hollow tube of aluminum, titanium, or alloys thereof. In an aspect, at least one of the telescoping segments is a hollow tube of load-bearing plastic or polymer. For example, at least one of the telescoping segments can be a hollow tube of carbon fiber-epoxy resin composite.

In an aspect, at least one of the two or more extendable and retractable load-bearing feet 1325 has a pivot at a connection to the linear rod 1305. For example, the at least one of the two or more extendable and retractable load-bearing feet having the two or more telescoping segments can further include a pivot at a connection to the linear rod. In some embodiments, at least one first of the two or more extendable and retractable load-bearing feet includes the two or more telescoping segments and at least one second of the two or more extendable and retractable load-bearing feet includes the pivot at a connection to the linear rod. In an aspect, at least one of the two or more extendable and retractable load-bearing feet 1325 is rotatable. For example at least one of the two or more extendable and retractable feet can be rotatable around a pivot at a connection to the linear rod.

In some embodiments, at least one of the two or more extendable and retractable load-bearing feet 1325 has a pivot at a connection to the linear rod 1304 and one or more additional pivots along a length of the at least one of the two or more extendable and retractable load-bearing feet 1325. In some embodiments, at least one of the two or more extendable and retractable load-bearing feet 1325 includes an additional foot pad at the distal end of the at least one of the two or more extendable and retractable load-bearing feet 1325. For example, the foot pad can include a rubber pad or cup attached to the distal end of the extendable and retractable load-bearing foot. For example, a rubber pad or cup can be added to prevent slippage of the extendable and retractable load-bearing feet on a slick walking surface. In an aspect, the foot pad includes a pivot connection to the extendable and retractable load-bearing foot. For example, the foot pad can include a pivot connection that allows it to pivot relative to movement of the extendable and retractable load-bearing foot to which it is attached.

In some embodiments, the two or more extendable and retractable load-bearing feet 1325 are operable to make the walking aid 1300 free-standing. For example, the two or more extendable and retractable load-bearing feet are operable to be moved into a position relative to one another that provides a stable platform for holding the walking aid in a near vertical position. In an aspect, a walking aid includes two or more extendable and retractable load-bearing feet including two or more telescoping segments operable to make the walking aid free-standing. In an aspect, a walking aid includes two or more extendable and retractable load-bearing feet including a pivot connection to a linear rod and two or more telescoping segments operable to make the walking aid free-standing. In an aspect, a walking aid includes two or more extendable and retractable load-bearing feet including a pivot connection to a linear rod and one or more additional pivots along the length of the two or more extendable and retractable load-bearing feet and operably to make the walking aid free-standing.

In some embodiments, at least one of the two or more extendable and retractable load-bearing feet 1325 is configured to retract for storage into an interior portion of the linear rod 1305. For example, the linear rod can include an interior portion constituting a compartment or hollow space inside a portion of the linear rod into which the extendable and retractable load-bearing feet can be retracted for storage. For example, the two or more extendable and retractable load-bearing feet can be attached at one end to a motor including pistons and/or pulleys that are capable of pushing and pulling the two or more extendable and retractable load-bearing feet out of and into the interior portion of the linear rod. In some embodiments, a user-activated control mechanism associated with the hand grip e.g., a button or switch, is used to initiate pushing or pulling of the two or more extendable and retractable load-bearing feet into or out of the interior portion of the linear rod.

In some embodiments, at least one of the two or more extendable and retractable load-bearing feet 1325 is configured to retract for storage on a side of the linear rod 1305. For example, the two or more extendable and retractable load-bearing feet can be configured retract or folded up for storage along the outer surface of the linear rod. In some embodiments, a user-activated control mechanism associated with hand grip, e.g., a button, is used to initiate pivoting or unfolding of the two or more extendable and retractable load-bearing feet.

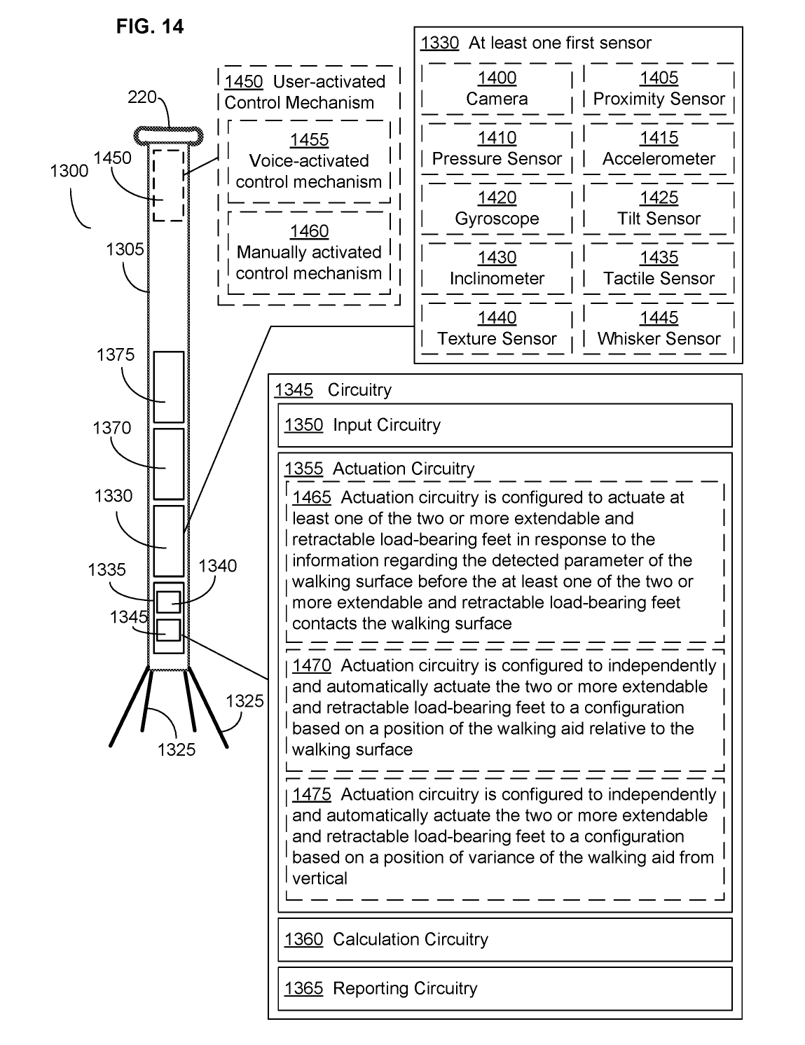

FIG. 14 illustrates further aspects of a walking aid such as shown in FIG. 13. Walking aid 1300 includes at least one first sensor 1330 configured to detect a parameter of a walking surface in proximity to the second end of the linear rod 1305. In an aspect, the parameter of the walking surface includes at least one of a surface type, a friction coefficient, a roughness, a slope, an edge, a height, a distance, or a bump. In an aspect, the at least one first sensor 1330 is incorporated into an interior portion of linear rod 1305. For example, an accelerometer, tilt sensor, or inclinometer can be incorporated into an interior portion of the linear rod. In an aspect, the at least one first sensor 1330 is incorporated onto an outer surface of linear rod 1305. For example, a camera or proximity sensor can be attached to an outer surface of the linear rod. In an aspect, the at least one first sensor 1330 is incorporated into hand grip 1320. In some embodiments, the at least one first sensor 1330 is associated with at least one of the two or more extendable and retractable load-bearing feet 1325. For example, that portion of an extendable and retractable load-bearing foot that makes contact with the walking surface can include one or more sensors, e.g., one or more pressure sensors.

In an aspect, the at least one first sensor 1330 includes at least one of a camera 1400, a proximity sensor 1405, or a pressure sensor 1410. The at least one camera, proximity sensor, and/or pressure sensor includes circuitry configured to detect a parameter of a walking surface in proximity to the second end of the linear rod and to transform the detect parameter of the walking surface into a first sensor output. In an aspect, the at least one first sensor 1330 includes at least one of an accelerometer 1415 or a gyroscope 1420. The at least one accelerometer or gyroscope includes circuitry configured to detect a parameter of a walking surface in proximity to the second end of the linear rod and to transform the detected parameter of the walking surface into a first sensor output. In an aspect, the at least one first sensor 1330 includes at least one of a tilt sensor 1425, an inclinometer 1430, a tactile sensor 1435, or a texture sensor 1440. The at least one tilt sensor, inclinometer, tactile sensor, and/or texture sensor includes circuitry configured to detect a parameter of the walking surface in proximity to the second end of the linear rod and to transform the detected parameter of the walking surface into a first sensor output. In an aspect, the at least one first sensor 1330 includes at least one whisker sensor 1445 including circuitry configured to detect a parameter of the walking surface in proximity to the second end of the linear rod and to transform the detected parameter of the walking surface into a first sensor output. Non-limiting aspects of cameras, proximity sensors, pressure sensors, accelerometers, gyroscopes, tilt sensor, inclinometers, tactile sensors, texture sensors, and whisker sensors has been described above herein. Returning to FIG. 14, walking aid 1300 includes a computing component 1335 including a microprocessor 1340 and circuitry 1345. In an aspect, microprocessor 1340 includes a central processing unit for controlling one or more functions of the walking aid. The controller further includes a system memory and a system bus that couples various system components including the system memory to the microprocessor. The microprocessor can include a processing unit, a central processing unit (CPU), a digital signal processor (DSP), an application-specific integrated circuit (ASIC), a field programmable gate entry (FPGA), or the like, or any combinations thereof, and can include discrete digital or analog circuit elements or electronics, or combinations thereof. In an aspect, the controller includes one or more ASICs having a plurality of pre-defined logic components. In an aspect, the controller includes one or more FPGA having a plurality of programmable logic commands.

Computing component 1335 further includes circuitry 1345. In an aspect, circuitry 1345 includes circuitry configured to execute one or more instructions for operating components of the walking aid, e.g., the at least one first sensor, the at least one second sensor, the reporting device, and the two or more extendable and retractable load-bearing feet. In an aspect, circuitry 1335 includes circuitry configured to execute one or more instructions for operating any or all other components incorporated into the walking aid, e.g., a user-activated control mechanism, a user interface, a receiver, a transmission unit, at least one third sensor, an actuator, a power source, a mechanical energy harvester, and/or a warning system. In an aspect, the controller includes circuitry to execute one or more instructions for receiving information regarding the detected parameter of the walking surface from the at least one first sensor and circuitry to execute one or more instructions for actuating at least one of the two or more extendable and retractable load-bearing feet in response to the information regarding the detected parameter of the walking surface.

Walking aid 1300 includes user-activated control mechanism 1450. In an aspect, a walking aid includes a user-activated control mechanism to control extension and retraction of the two or more extendable and retractable load-bearing feet. In an aspect, the user-activated control mechanism 1450 is a voice-activated control mechanism 1455. For example, the walking aid can include a microphone and circuitry configured to receive, process, and transmit a voice command to the controller. For example, the voice commands can include “on,” “off,” “extend,” “retract,” and the like. In an aspect, the user-activated control mechanism 1450 is a manually activated control mechanism 1460. For example, the walking aid can include one or more buttons or other pressure sensitive pads and circuitry configured to receive and process a manual activation command, e.g., pushing of a button. In an aspect, at least a portion of the user-activated control mechanism is incorporated into the hand grip disposed in proximity to the first end of the linear rod. For example, the hand grip can include a microphone for receiving a voice command. For example, the hand grip can include one or more pressure-sensitive buttons or pads for sending a manual activation command.

Computing component 1335 includes circuitry 1345 including actuation circuitry 1355 configured to actuate at least one of the two or more extendable and retractable load-bearing feet in response to the information regarding the detected parameter of the walking surface. In some embodiments, as shown in block 1465, the actuation circuitry is configured to actuate at least one of the two or more extendable and retractable load-bearing feet in response to the information regarding the detected parameter of the walking surface before the at least one of the two or more extendable and retractable load-bearing feet contacts the walking surface. For example, the controller can receive information from the at least one first sensor, e.g., a camera or proximity sensor, regarding a parameter of the walking surface, e.g., a curb edge or break in the sidewalk, and the actuation circuitry can actuate at least one of the two or more extendable and retractable load-bearing feet before the at least one of the two or more extendable and retractable load-bearing feet contacts the walking surface. In an aspect, the actuation circuitry of block 1465 is configured to execute one or more instructions for actuating at least one of the two or more extendable and retractable load-bearing feet in response to information regarding the detected parameter of the walking surface before the at least one of the two or more extendable and retractable load-bearing feet contacts the walking surface.

In an aspect, circuitry 1345 includes actuation circuitry configured to actuate extension or retraction of at least one of the two or more extendable and retractable load-bearing feet in response to the information regarding the detected parameter of the walking surface before the at least one of the two or more extendable and retractable load-bearing feet contacts the walking surface. In an aspect, the actuation circuitry is configured to execute one or more instructions for actuating extension or retraction of at least one of the two or more extendable and retractable load-bearing feet in response to the information regarding the detected parameter of the walking surface before the at least one of the two or more extendable and retractable load-bearing feet contacts the walking surface.

In an aspect, circuitry 1345 includes actuation circuitry configured to independently and automatically actuate the two or more extendable and retractable load-bearing feet. For example, the controller can receive information from the at least one first sensor, e.g., a proximity sensor, regarding a parameter of the walking surface, e.g., a stepped walking surface, and the actuation circuitry can independently and automatically actuate the two or more extendable and retractable load-bearing feet to create a stable support for the user. For example, the controller can include actuation circuitry configured to independently and automatically actuate each of the two of more extendable and retractable load-bearing feet. For example, the controller can include actuation circuitry configured to actuate one of the two or more extendable and retractable load-bearing feet independently of each of the other two or more extendable and retractable load-bearing feet. In an aspect, the actuation circuitry is configured to execute one or more instructions for independently and automatically actuating the two or more extendable and retractable load-bearing feet.

In some embodiments, as shown in block 1470, the actuation circuitry is configured to independently and automatically actuate the two or more extendable and retractable load-bearing feet to a configuration based on a position of the walking aid relative to the walking surface. For example, the controller can receive information from the at least one first sensor, e.g., a proximity sensor, regarding the position of the walking aid relative to the walking surface, e.g., height and distance away from a step associated with a stepped walking surface, and the actuation circuitry can independently and automatically actuate the two or more extendable and retractable load-bearing feet to create a stable support for the user. In an aspect, the actuation circuitry of block 1470 is configured to execute one or more instructions for independently and automatically actuating the two or more extendable and retractable load-bearing feet to a configuration based on a position of the walking aid relative to the walking surface.

In some embodiments, as shown in block 1475, the actuation circuitry is configured to independently and automatically actuate the two or more extendable and retractable load-bearing feet to a configuration based on a position of variance of the walking aid from vertical. For example, the controller can receive information from at least one accelerometer regarding a tilt or inclination of the walking aid relative to the walking surface and the actuation circuitry can independently and automatically actuate the two or more extendable and retractable load-bearing feet to create stable support for the user. In an aspect, the actuation circuitry of block 1475 is configured to execute one or more instructions for independently and automatically actuating the two or more extendable and retractable load-bearing feet to a configuration based on a position of variance of the walking aid from vertical.

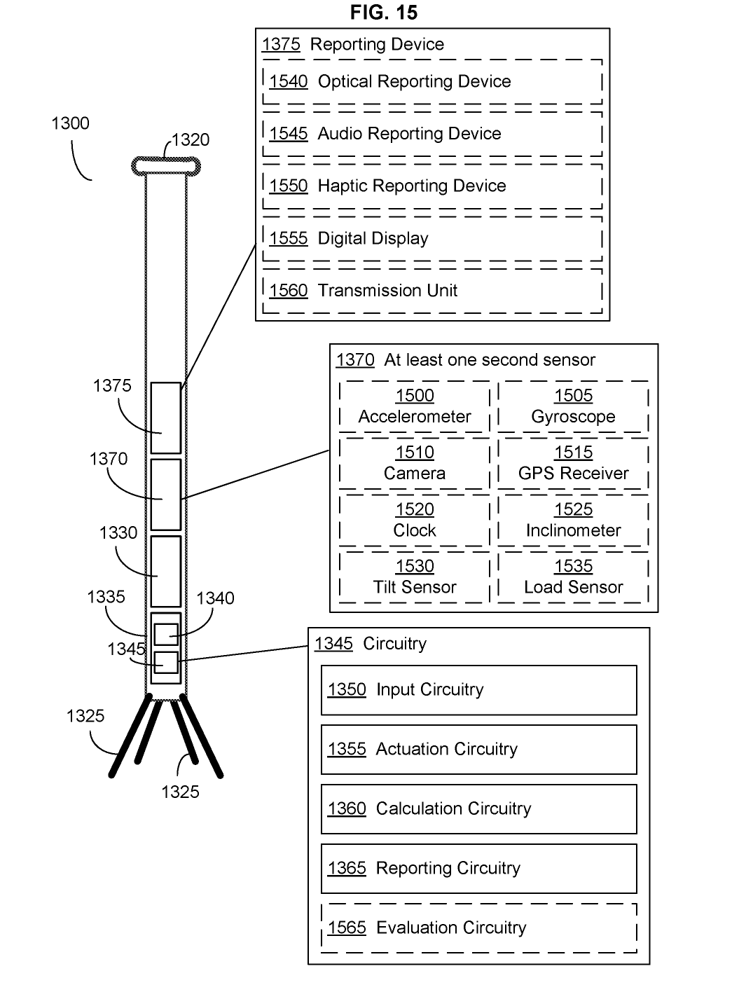

FIG. 15 illustrates further aspects of a walking aid. Walking aid 1300 includes at least one second sensor 1370 configured to detect a parameter of the walking aid 1300 and to transform the detected parameter of the walking aid 1300 into a second sensor output. In an aspect, the parameter of the walking aid comprises at least one of movement, speed, location, tip motion of the walking aid, position of the walking aid relative to the walking surface, or tilt angle of the walking aid relative to the walking surface. In an aspect, the at least one second sensor 1370 measures the movement of the walking aid in three-dimensional space. In an aspect, the at least one second sensor 1370 measures the movement of the walking aid relative to a walking surface. In an aspect, the at least one second sensor 1370 measures the movement of the walking aid relative to the user.

In an aspect, the at least one second sensor 1370 includes at least one of an accelerometer 1500, a gyroscope 1505, a camera 1510, a GPS receiver 1515, or a clock 1520. In an aspect, the at least one second sensor 1370 includes at least one of an inclinometer 1525, a tilt sensor 1530, or a load sensor 1535. In an aspect, the at least one second sensor includes at least one of a tactile sensor, a whisker sensor, a proximity sensor, and/or a pressure sensor. Non-limiting aspects of sensors have been described above herein.

In an aspect, the at least one second sensor provides an indication of how often a user is using the walking aid, as indicated by accelerometers, motion detectors, and clocks. In an aspect, the at least one second sensor provides an indication of where a user is going with the walking aid, as indicated by a GPS receiver associated with the walking aid or access to a local positioning system. In an aspect, the at least one second sensor provides an indication as to a path (e.g., a straight versus weaving path) a user is taking with the walking aid. In an aspect, the at least one second sensor provides an indication as to how fast a user is traveling with the walking aid, as indicated by a speedometer sensor. In an aspect, the at least one second sensor provides an indication of the quality of the walking, e.g., is the user weaving, shuffling, leaning to one side, or other.

In an aspect, at least one second sensor 1370 is configured to determine movement of the walking aid before, during, and after it loses contact with the walking surface. For example, an accelerometer in combination with a pressure sensor can be used to detect when the walking aid loses contact with the walking surface and what the movement of the walking aid looks like after losing contact. In an aspect, a tilt sensor or inclinometer in combination with accelerometers can measure the pitch and roll of the walking aid relative to the walking surface before, during, and after the walking aid loses contact with the walking surface. In an aspect, a combination of two or more sensors is used to measure the height of the walking aid. For example, a combination of 3D gyroscopes, 3D accelerometers, 3D magnetometers, and a barometric altimeter can be used as a height sensor. See, e.g., Tanigawa et al. (2008) “Drift-free dynamic height sensor using MEMS IMU aided by MEMS pressure sensor” Proceedings of the 5^{th} Workshop on Positioning, Navigation, and Communication 2008, pp. 191-196, which is incorporated herein by reference.

In an aspect, at least one second sensor 1370 is configured to detect tip motion at the first end or the second end of the linear rod after initial contact with the walking surface. For example, a 2-axis inclinometer with accelerometers can be used to measure a tipping motion, tilt, or variance from vertical of the walking aid relative to a walking surface. Non-limiting examples of inclinometers and accelerometers have been described above herein.

Returning to FIG. 15, walking aid 1300 further includes reporting device 1375. In an aspect, reporting device 1375 is configured to provide a report based on the calculated gait value. For example, an audio, haptic, or optical reporting device can be used to report information regarding the calculated gait value. In an aspect, the user is an individual (e.g., a healthcare provider or caretaker) monitoring a user of the walking aid. In some embodiments, reporting device 1375 is configured to report information to an external device. For example, the reporting device can include a transmission unit and antenna for transmitting information to an external device, e.g., a smart phone. The report may include a visual report, an audible report, or a haptic report based on the calculated gait value. In some embodiments, reporting device 1375 is configured to directly report information to a user, i.e., the subject using the walking aid. In some embodiments, reporting device 1375 is incorporated into or onto the handgrip 1320. In some embodiments, reporting device 1375 is incorporated into or onto linear rod 1305 of walking aid 1300. In an aspect, the reporting device comprises at least one of an optical reporting device 1540, an audio reporting device 1545, or a haptic reporting device 1550.

In some embodiments, reporting device 1375 includes optical reporting device 1540. In an aspect, the optical reporting device includes one or more light indicators. For example, the reporting device can include one or more lights, e.g., light-emitting diodes (LEDs), configured to light up in response to the information regarding the calculated gait value. In an aspect, the optical reporting device includes one or more color-coded lights. For example, the reporting device can include LEDs of different colors and a coding system. For example, a signal from a green LED can indicate that the calculated gait value falls within a range of acceptable gait values while a red LED can indicate that the calculated gait value fails to fall within a range of acceptable gait values.

In some embodiments, reporting device 1375 includes audio reporting device 1545 including at least one speaker. For example, the reporting device can include an audio reporting device that emits an audible signal in response to the information regarding the calculated gait value. For example, the audio reporting device can emit a warning sound, e.g., a beeping sound, if the calculated gait value fails to fall within a range of acceptable gait values. For example, the audio reporting device can emit one or more spoken words indicating whether the calculated gait value falls within a range of acceptable gait values. Electronic sound chips and/or sound cards for use as an audio reporting device are available from commercial sources (from, e.g., STMicroelectronics, Geneva, Switzerland).

In some embodiments, reporting device 1375 includes a haptic reporting device 1550. For example, the reporting device can include a haptic reporting device that emits a haptic signal, e.g., a vibrational signal, in response to the information regarding the calculated gait value. In an aspect, the haptic reporting device 1550 is incorporated into or onto a surface of the hand grip 1320 of walking aid 1300. In an aspect, the haptic reporting device 1550 is incorporated into or onto a surface of the linear rod 1305 of walking aid 1300. For example, the haptic reporting device can include a vibrational motor (e.g., a coin or pancake vibration motor, from, e.g., Precision Microdrives Ltd, London, UK) that might be used to let the nursing mother know that sufficient breast milk has been expressed.

In some embodiments, reporting device 1375 comprises a digital display 1555. For example, the digital display can include an OLED, LCD, or touch screen display with a screen intended to display information. The information can include, but is not limited to, information regarding a property of the walking surface, information regarding a property of the walking aid, a calculated gait value, an evaluate condition of a user, a physiological parameter of the user, an alert or warning, a map, or information from another device (e.g., a wearable device worn by the user).

In some embodiments, at least one of the optical reporting device, audio reporting device, haptic reporting device, or digital display are associated with or incorporated into a user interface of the walking aid. In some embodiments, at least one of the optical reporting device, audio reporting device, haptic reporting device, or digital display are associated with or incorporated into a warning system of the walking aid.

In some embodiments, reporting device 1375 comprises a transmission unit 1560, including an antenna. In an aspect, the transmission unit 1560 comprises an optical transmission unit. In an aspect, the transmission unit 1560 is a radiofrequency transmission unit. A “transmission unit,” as used herein, can be one or more of a variety of units that are configured to send and/or receive signals, such as signals carried as electromagnetic, radio, sonic, or optical waves. A transmission unit generally includes at least one antenna and associated circuitry. A transmission unit can be operably connected to computing component 1335 and/or can include its own a processor and/or memory component. A transmission unit can be operably connected to an energy source, such as a battery. A transmission unit can include an energy harvesting unit, such as a unit configured to obtain energy from electromagnetic waves. Other non-limiting aspects of transmission units have been describe above herein.

In an aspect, transmission unit 1560 includes circuitry configured to transmit a signal to a remote computing device, the signal including information regarding the calculated gait value. For example, the transmission unit can transmit a signal to a tablet, a laptop, or tabletop computing device, the signal including information regarding the calculated gait value. In an aspect, transmission unit 1560 includes circuitry configured to transmit a signal to a mobile communication device, the signal including information regarding the calculated gait value. For example, the transmission unit can include circuitry configured to transmit the calculated gait value to a mobile communication device, e.g., a cellular or smart phone. For example, the transmission unit including the antenna can be connected through a wireless radiofrequency communication link, e.g., Bluetooth or WiFi, to a smart phone. For example, the transmission unit of the walking aid can be configured to sync with an external device, e.g., a smart phone, to transfer information, e.g., the calculated gait value. In an aspect, the transmission unit is configured to transmit the calculated gait value to a dedicated handheld device. For example, a dedicated handheld device can include a transmission unit and antenna for communicating with the walking aid 1300, a user interface, e.g., a display, microphone, or haptic interface, for displaying and/or notifying a user during a walking event, and a computing component to display and save information received from the walking aid. In an aspect, the remote computing device and/or the mobile communication device includes a program, set of instructions, and/or an application configured to receive information from the walking aid, process the received information to evaluate a condition of the user based on the received calculated gait value and display the received calculated gait value and/or the evaluate medical or mental condition of the user. In an aspect, the transmission unit includes circuitry configured to transmit the calculated gait value to a cloud computing device.

Returning to FIG. 15, computing component 1335 of walking aid 1300 includes circuitry 1345 including input circuitry 1350, actuation circuitry 1355, calculation circuitry 1360, and reporting circuitry 1365. The circuitry is intended to receive sensor output data and actuate the extendable and retractable load-bearing feet and/or calculate a gait value based on the received sensor output. The circuitry is further configured to transmit a signal including information regarding the calculated gait value to the reporting device.

Computing component 1335 includes input circuitry 1350 configured to receive the second sensor output including information regarding the detected parameter of the walking aid. The second sensor output received from one or more accelerometers, gyroscopes, cameras, a GPS receiver, tilt sensor, inclinometers, and clocks associated with the walking aid includes information regarding a detected parameter of the walking aid. The detected parameter of the walking aid can include at least one of movement, speed, location, tip motion of the walking aid, position of the walking aid relative to the walking surface, or tilt angle of the walking aid relative to the walking surface. In an aspect, the second sensor output received by the input circuitry 1350 provides an indication of how often a user is using the walking aid and for how long, as indicated by data from accelerometers, motion detectors, and clocks associated with the walking aid. In an aspect, the second sensor output received by the input circuitry 1350 provides an indication of where a user is going and how far with the walking aid, as indicated by data from a GPS receiver associated with the walking aid or through access to a local positioning system. In an aspect, the second sensor output received by the input circuitry 1350 provides an indication as to a path (e.g., a straight versus weaving path) a user is taking with the walking aid. In an aspect, the second sensor output received by the input circuitry 1350 provides an indication as to how fast a user is traveling with the walking aid, as indicated by data from a speedometer sensor. In an aspect, the second sensor output received by the input circuitry 1350 provides an indication of the quality of the walking, e.g., is the user weaving, shuffling, leaning to one side, or other, as indicated from data from accelerometers, gyroscopes, tilt sensors, and/or inclinometers associated with the walking aid. In an aspect, the second sensor output received by the input circuitry 1350 includes one or more images captured with a digital camera associated with the walking aid, the one or more images providing information regarding a gait of a user.

Computing component 1335 includes calculation circuitry 1360 configured to calculate a gait value based on the received information regarding the detected parameter of the walking aid 1300. In an aspect, the calculation circuitry includes one or more algorithms configured to calculate the gait value based on the received information regarding the detected parameter of the walking aid. The one or more algorithms can include machine learning algorithms, non-limiting examples of which include linear regression, logistic regression, decision tree, SVM (Support Vector Machine), Naïve Bayes, KNN (K-nearest neighbors), Dimensionality Reduction Algorithms, artificial neural networks, and the like. In an aspect, the calculation circuitry includes non-transitory computer readable medium including computer readable instructions for calculating a gait value based on the received information regarding the detected parameter of the walking aid. In an aspect, the calculation circuitry includes modeling capabilities configured to generate a motion model of the walking aid relative to the user and the walking surface over time and to calculate the gait value based on the motion model. In an aspect, the calculation circuitry is configured access look-up tables, feature maps, training sets, and/or databases including reference data points and/or motion models for comparison with the received information regarding the detected parameter of the walking aid.

The calculated gait value can be an overall assessment of the user's gait based on the information regarding the detected parameter of the walking aid, i.e., the movement or lack thereof of the walking aid relative to the user and/or the walking surface. The calculation of the gait value can take into consideration the pace, rhythm, intra-step variability, and asymmetry of the user's gait. In an aspect, the gait value can take into consideration one or more spatiotemporal subcomponents of gait including at least one of velocity (distance traversed divided by time), cadence (steps per minute; stepping frequency), double support time (period within a stride during which both feet are simultaneously on the ground), single limb support time (time elapsed with only one foot in contact with the ground), stride length (distance in anterior-posterior direction between successive point of contact of the same foot (i.e., two steps)), step length (distance in anterior-posterior direction between successive points of contact of opposite foot (i.e., one step)), stride time (time elapsed between successive points of contact of the same foot (i.e., two steps)), step time (time elapsed between successive points of contact of opposite foot (i.e., one step)), step width (distance in medio-lateral direction between two feet (i.e., base of support)), swing time (time elapsed with one foot in the air and one on the ground), variability (calculated coefficient of variation or standard deviation of one of the above, e.g., step length, stride length, step time, stride time, and/or step width; and asymmetry (differences between symmetrical spatiotemporal characteristics of left and right leg during gait)).

In an aspect, the calculated gait value provides an indication of the posture, balance, and/or mobility of the user. For example, if the user is only periodically using the walking aid, this may indicate a change in mobility. In some instances, the change in mobility might be improved mobility of the user such that the walking aid is no longer needed, thus accounting for the reduced frequency of use. In some instances, the change in mobility might indicate reduced mobility, either through a physical condition (e.g., increased joint, muscle, and/or nerve pain, or worsening heart and/or lung disease) or a mental condition (e.g., worsening depression and decreased desire to move about). For example, if the user is dropping the walking aid more frequently, this may indicate a change in grip strength or balance and a possible increased risk of falling. For example, if the at least one second sensor indicates that the walking aid is being held at an awkward angle, this may indicate a change in posture.

Computing component 1335 includes reporting circuitry 1365 configured to transmit a signal to the reporting device in response to the calculated gait value. In some embodiments, reporting circuitry 1365 is configured to transmit a signal to a digital display associated with the walking aid in response to the calculated gait value. The digital display associated with the walking aid can represent the calculated gait value as text, numbers, images, graphs, plots, and/or tables. In some embodiments, reporting circuitry 1365 is configured to transmit a signal to at least one of an optical reporting device, an audio reporting device, or a haptic reporting device in response to the calculated gait value. In some embodiments, reporting circuitry 1365 is configured to transmit a signal to a transmission unit associated with the walking aid, which in turn reports the calculated gait value to a computing device (e.g., mobile communication device or tablet, laptop, or desktop computer) or a network.

In some embodiments, the computing component 1335 includes evaluation circuitry 1565 configured to evaluate a condition of the user based on a comparison of the calculated gait value with at least one reference gait value; and the reporting circuitry 1365 is configured to transmit a signal to the reporting device 1375 in response to the evaluated condition of the user. In an aspect, the computing component 1335 of walking aid 1300 can include a database of stored reference gait values correlated with medical or mental conditions. In an aspect, the at least one reference gait value is one or more previously calculated gait values for the user. In an aspect, the at least one reference gait value is derived from one or more matched individuals. For example, the reference gait value may be matched to a user based on age, weight, gender, height. For example, the reference gate value may be matched to a user based on a medical condition or mental individual. In an aspect, the calculated gait value when compared with the at least one reference gait value indicates a new or worsening medical and/or mental condition, e.g., heart disease, spinal cord compression associated myelopathy (loss of balance, grip, and increased clumsiness), chronic obstructive pulmonary disease, arthritis, osteoporosis, dementia, stroke, cognitive decline, depression, multiple sclerosis, and the like. Changes in gait may be indicative of arthritic joints or peripheral neuropathies. A spastic gait may be indicative of vascular injuries to the brain (strokes) or tumors. A vestibular gait may be indicative of a dysfunction in the inner ear.

In an aspect, the calculated gait value when compared with the at least one reference gait value indicates improvement or lack thereof as a result of an exercise and/or physical therapy/occupational therapy program. For example, the walking aid may be used to monitor improvements and/or progression of gait in response to medication, psychotherapy, physical therapy, or exercise. For example, the walking aid may be used to monitor improvements and/or progression of gait in response to alternative medicine therapies, e.g., massage, acupuncture, naturopathic medicines, chiropractic therapy, and the like.

In an aspect, the reporting circuitry 1365 of computing device 1335 is configured to transmit a signal to the reporting device 1375 in response to the evaluated condition of the user. In an aspect, reporting circuitry 1365 is configured to transmit a signal to at least one of optical reporting device 1540, audio reporting device 1545, haptic reporting device 1550, or digital display 1555 in response to the evaluated condition of the user. In an aspect, reporting circuitry 1365 is configured to transmit a signal in response to the evaluated condition of the user to a warning system associated with the walking aid 1300 and operably coupled to the computing component 1335 and including at least one a haptic reporting device, an audio reporting device, an optical reporting device, or digital display. In an aspect, reporting circuitry 1365 is configured to transmit a signal in response to the evaluated condition of the user to a user interface associated with the walking aid 1300 and operably coupled to the computing component 1335 and including at least one a haptic reporting device, an audio reporting device, an optical reporting device, or digital display. In an aspect, the reporting circuitry 1365 is configured to transmit a signal in response to the evaluated condition of the user to transmission unit 1560 associated with walking aid 1300. In some embodiments, the transmission unit 1560 transmits the information regarding the evaluated condition of the user to an external computing device, e.g., a dedicated hand-held device, a mobile communication device, a laptop, tablet, or desktop computing device, or a cloud computing device. In some embodiments, the transmission unit 1560 transmits the information regarding the evaluated condition of the user to an external computing device associated with a network, e.g., a healthcare network. In some embodiments, the transmission unit 1560 transmits the information regarding the evaluated condition of the user to an external computing device associated with the user's electronic medical record. In some embodiments, the transmission unit 1560 transmits the information regarding the evaluated condition of the user to an external computing device associated with the user's electronic medical record associated with a care provider or medical practitioner.

In some embodiments, the sensed movement of the walking aid relative to the user and the walking surface can inform the elongation and retraction of the extendable and retractable load-bearing feet. In some embodiments, computing component 1335 includes circuitry 1345 including input circuitry 1350 configured to receive the second sensor output including the information regarding the detected parameter of the walking aid 1300 and actuation circuitry 1355 is configured to actuate at least one of the two or more extendable and retractable load-bearing feet 1325 in response to the information regarding the detected parameter of the walking aid 1300. For example, the controller can include circuitry to actuate at least one of the two or more extendable and retractable load-bearing feet in response to detecting a substantial tilt in the walking aid relative to the walking surface. In an aspect, the input circuitry 1350 is configured to execute one or more instructions for receiving information regarding the detected parameter of the walking aid 1300 from the at least one second sensor 1370 and the actuation circuitry 1355 is configured to execute one or more instructions for actuating at least one of the two or more extendable and retractable load-bearing feet 1325 in response to the information regarding the detected parameter of the walking aid 1300.

In some embodiments, computing component 1335 includes circuitry 1345 including actuation circuitry 1355 configured to actuate the at least one of the two or more extendable and retractable load-bearing feet 1325 in response to movement of the walking aid 1300 after it loses contact with the walking surface. In an aspect, the actuation circuitry is configured to execute one or more instructions for actuating the at least one of the two or more extendable and retractable load-bearing feet 1325 in response to movement of the walking aid 1300 after it loses contact with the walking surface. In an aspect, computing component 1335 includes circuitry 1345 including actuation circuitry 1355 configured to actuate extension or retraction of the at least one of the two or more extendable and retractable load-bearing feet 1325 in response to movement of the walking aid 1300 after it loses contact with the walking surface. In an aspect, the actuation circuitry 1355 is configured to execute one or more instructions for actuating extension or retraction of the at least one of the two or more extendable and retractable load-bearing feet 1325 in response to movement of the walking aid 1300 after it loses contact with the walking surface. In an aspect, computing component 1335 includes circuitry 1345 including actuation circuitry 1355 configured to actuate the at least one of the two or more extendable and retractable load-bearing feet 1325 to orient said at least one of the two or more extendable and retractable load-bearing feet 1325 relative to an orientation of the walking aid 1300. In an aspect, the actuation circuitry 1355 is configured to execute one or more instructions for actuating the at least one of the two or more extendable and retractable load-bearing feet 1325 to orient said at least one of the two or more extendable and retractable load-bearing feet 1325 relative to an orientation of the walking aid 1300.

In some embodiments, the walking aid 1300 includes an actuator operably coupled to the computing component 1335 and the actuation circuitry 1355 and configured to actuate the two or more extendable and retractable load-bearing feet 1325. In an aspect, the actuator produces a rotational motion (e.g., rotating at least a portion of an extendable and retractable load-bearing foot around a pivot point). In an aspect, the actuator produces a linear motion (e.g., extending and retracting the two or more telescoping segments of the extendable and retractable load-bearing foot). In an aspect, the actuator includes at least one of a spring, a motor, a pneumatic actuator, or a hydraulic actuator. In an aspect, the actuator includes at least one of a linear actuator, a solenoid, a muscle wire, or a mechanical actuator. In an aspect, the actuator comprises a combination of a spring, a motor, a pneumatic actuator, a hydraulic actuator, a linear actuator, a solenoid, and/or a muscle wire. Non-limiting aspects of actuators has been described above herein.

In an aspect, the walking aid 1300 includes a power source for powering any or all components of the walking aid. In an aspect, the walking aid includes one or more disposable batteries, e.g., cells, buttons, thin-film batteries, or microbatteries. For example, the components of the walking aid can be powered by a conventional battery, e.g., a disposable 9 volt battery. Non-limiting examples of disposable batteries include zinc-carbon, alkaline, lithium, zinc-chloride, zinc-air, or silver-oxide batteries. In an aspect, the one or more batteries include one or more rechargeable batteries. For example, the components of the walking aid can be powered by one or more rechargeable lithium-ion batteries. Non-limiting examples of rechargeable batteries include nickel-cadmium, nickel-zinc, nickel metal hydride, silver-zinc, or lithium ion.

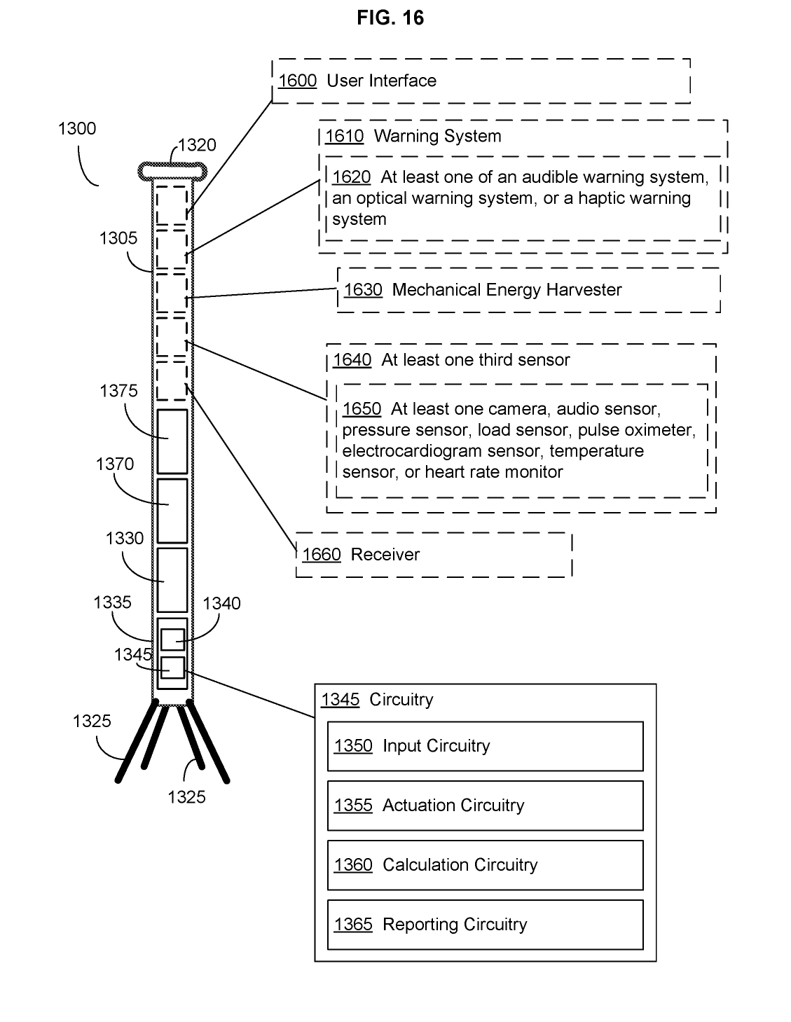

FIG. 16 illustrates further aspects of a walking aid. In some embodiments, walking aid 1300 includes user interface 1600 for input/output of information. The user interface can include one or more input components and/or output components for use by a user to interface with the walking aid. In an aspect, the user interface can include an input component (e.g., a microphone, button, or switch) for controlling one or more functions of the walking aid. For example, the user interface can include an input component that is part of a user-activated control mechanism to control extension and retraction of the two or more extendable and retractable load-bearing feet. Non-limiting examples of input components include buttons, switches, triggers, keypads, touch-screens, microphones, dials, or the like. In an aspect, the user interface can include an output component for providing information to a user. For example, an output component, e.g., a flashing light emitting diode (LED), can be used as part of a warning system configured to alert a user. Non-limiting examples of output components include audio speakers, light emitting diodes or other light generating devices, or a haptic device. In some embodiments, user interface 1600 is part of a user-activated control mechanism to control extension and retraction of the two or more extendable and retractable load-bearing feet 1325. For example, one or more buttons on the hand grip can be used to manually control extension and retraction of the two or more extendable and retractable load-bearing feet. In some embodiments, the user interface 1600 is part of a warning system 1610 operably coupled to the controller 1335 and configured to alert a user. For example, a light emitting diode on the hand grip can be used to alert a user to a hazard associated with the walking surface, e.g., a crack in a sidewalk or the edge of a curb. In some embodiments, user interface 1600 includes a display. For example, the user interface can include an OLED, LCD, or touch screen display with a screen intended to display information. The information can include, but is not limited to, information regarding a property of the walking surface, information regarding a property of the walking aid, a physiological parameter of the user, an alert or warning, a map, information from another device (e.g., a wearable device worn by the user). In an aspect, the user interface 1600 is associated with the hand grip 1320 and/or the linear rod 1305 of the walking aid 1300. In an aspect, the user interface 1600 is operably coupled to the reporting device 1375. In an aspect, the user interface 1600 is part of the reporting device 1375. In an aspect, the reporting device 1375 is part of the user interface.

In some embodiments, the walking aid 1300 includes warning system 1610 operably coupled to computing component 1335 and configured to alert a user. For example, the walking aid can include a warning system that alerts a user to a dangerous walking surface or situation. In an aspect, warning system 1610 comprises at least one of an audible warning system, an optical warning system, or a haptic warning system 1620. In an aspect, the warning system includes a speaker, a soundcard, and circuitry configured to broadcast an audible alert in response to receiving a warning signal from the controller. For example, the warning system can include an audible alert, e.g., a beeping sound or a warning voice. In an aspect, the warning system includes one or more light sources, and circuitry configured to emit an optical alert in response to receiving a warning signal from the controller. For example, the warning system can include an optical alert, e.g., one or more flashing red LEDs. In an aspect, the warning system includes a vibrator and circuitry configured to vibrate at least a portion of the walking aid in response to receiving a warning signal from the controller. For example, the hand grip of the walking aid can include a haptic device, e.g., a vibrator, which vibrates the hand grip in response to a warning signal from the controller. Non-limiting aspects of a warning system are described above herein.

In an aspect, warning system 1610 is incorporated into the hand grip 1320 of the walking aid 1300. In an aspect, at least one of an audible warning system, an optical warning system, or a haptic warning system 1620 is incorporated into hand grip 1320. For example, the warning system can include one or more lights, e.g., LEDs, incorporated into the hand grip that flash on/off to alert the user. For example, the warning system can include a speaker incorporated into the hand grip that emits an audible sound, e.g., a beeping sound, to alert the user. For example, the warning system can include a haptic vibration device that vibrates the hand grip of the walking device to alert the user. Alternatively, the warning system can be incorporated into one or more other parts of the walking aid, e.g., into the linear rod or into at least one of the two or more extendable and retractable load bearing feet.

In some embodiments, the walking aid 1300 further includes a mechanical energy harvester 1630 configured to store electrical or mechanical energy to power actuation of the two or more extendable and retractable load-bearing feet 1325. In an aspect, the mechanical energy harvester stores mechanical energy generated when the walking aid periodically contacts the walking surface. For example, the intermittent mechanical energy generated when the walking aid contacts the walking surface can be converted into electrical energy through at least one of piezoelectric conversion, electro-active polymers (EAP) conversion, or electromagnetic conversion. For example, the mechanical energy harvester can include one or more piezoelectric materials, dielectric elastomers, and/or rotator generators. See, e.g., Yusuf et al. (2013) “Mechanical energy harvesting devices for low frequency applications: Revisited,” ARPN J Eng. App. Sci. 8:504-512, which is incorporated herein by reference. In an aspect, the mechanical energy harvester stores electrical or mechanical energy for powering one or more components of the walking aid. In an aspect, the mechanical energy harvester stores electrical or mechanical energy for powering an actuator, e.g., spring, a motor, a pneumatic actuator, a hydraulic actuator, or solenoid, to power actuation of the two or more extendable and retractable load-bearing feet 1325. In an aspect, power from a mechanical energy harvester is combined with another power source, e.g., a standard battery, to power the components of the walking aid.

In some embodiments, a walking aid such as described herein may further include additional sensors for monitoring a physiological condition of a user. In an aspect, the walking aid 1300 includes at least one third sensor 1640 operably coupled to the computing component 1345 and including circuitry configured to detect a physiological parameter of a user and to transform the detected physiological parameter of the user into a third sensor output, wherein the input circuitry 1350 is configured to receive the third sensor output including information regarding the detected physiological parameter of the user, and the reporting circuitry 1365 is configured to transmit a signal to the reporting device in response to the received information regarding the detected physiological parameter of the user. In some embodiments, the at least one third sensor 1640 is incorporated into the hand grip 1320 of walking aid 1300. For example, the walking aid can include one or more physiological sensor incorporated into the hand grip. In an aspect, the at least one third sensor 1640 includes at least one camera, audio sensor, pressure sensor, load sensor, pulse oximeter, electrocardiogram sensor, temperature sensor, or heart rate monitor, as shown in block 1650 of FIG. 16. For example, the at least one third sensor can include a camera (e.g., a digital camera) positioned to capture images of a portion of the user. The camera can be positioned to image the user's legs to monitor gait. The camera can be positioned to image the user's face or other area of exposed skin to assess coloring (e.g., flushed, ashen, purple, jaundiced, etc.). For example, the at least one third sensor can include an audio sensor, e.g., a microphone, to pick up sounds associated with the user and his/her use of the walking aid. For example, the audio sensor can be configured to detect speech (e.g., speech patterns and content, frequency and quality) as a means of monitoring a user's mental or medical state. For example, the audio sensor can be configured to detect breathing patterns (e.g., shortness of breath or wheezing with limited exertion). For example, the audio sensor can be configured to detect the sounds of the user's feet (e.g., foot or shoe-strike including shuffling) while walking with the walking aid. For example, the at least one third sensor can include a pressure sensor, e.g., in the hand grip, configured to measure the grip strength of a user. For example, the at least one third sensor can include a load sensor configured to measure how much of the user's body weight is being supported by the walking aid. For example, the at least one third sensor can include a pulse oximeter configured to measure the blood oxygenation or oxygen saturation of the user's blood. For example, the at least one third sensor can include a thermometer for monitoring the body temperature of the user. For example, the at least one third sensor can include a heart rate monitor attached to the hand grip of the walking aid and configured to monitor the heart rate of a user while using the walking aid.

Returning to FIG. 16, in some embodiments, the walking aid 1300 can further include receiver 1660. In an aspect, receiver 1660 includes circuitry configured to receive a transmitted signal from an external device. In some embodiments, the receiver includes circuitry configured to receive a transmitted signal from an external computing device. For example, the walking aid can include a receiver for receiving signals from at least one of a mobile communication device, a dedicated hand-held device, a computer (e.g., a laptop, tablet, or desktop computer), a network, a server, or other device or system. In some embodiments, the receiver includes circuitry to receive a transmitted signal from a wearable device, e.g., a fitness monitor, heart rate monitor, pedometer, or similar type of device. The information received can be added to or correlated with information incorporated into the first and second sensor outputs. The information received can be added to or correlated with information incorporated in a third sensor output including a detected physiological parameter of the user.

In an aspect, receiver 1660 is an optical receiver including circuitry configured to receive an optical signal from a remote optical signal generator configured for receiving optical signal transmission. In an aspect, receiver 1660 is a radiofrequency receiver including circuitry configured to receive a radiofrequency signal from an external radiofrequency signal generator. In an aspect, receiver 1660 includes circuitry configured to receive at least one of a radio signal, a magnetic signal, an acoustic signal, an electromagnetic signal, or an optical signal. The receiver can include at least one of an electromagnetic signal receiver, a magnetic signal receiver, a microwave signal receiver, an acoustic signal receiver, an optical signal receiver, or other receiver types. In an aspect, the receiver includes at least one antenna configured to intercept signals emitted from an external device (e.g., a computing device, a wearable device, and/or a remote signaling device). In an aspect, the receiver includes at least one filter, e.g., an electronic filter, a frequency filter, or a bandwidth filter, to separate specific signals from the external device from other signals in the environment. In an aspect, the receiver includes a digital signal processor. Other non-limiting aspects of a receiver are described above herein.

In some embodiments, receiver 1660 is configured to receive a wireless signal from a remote signaling device, e.g., one or more beacons. In an aspect, receiver 1660 is configured to receive a signal from a remote signaling device as a user approaches an environment. In an aspect, the receiver 1660 and/or an operably coupled computing component 1335 is configured to transform the received signal into information regarding a location of the walking aid 1300 in the environment, e.g., approaching a stepped surface. In some embodiments, the actuation circuitry 1355 of computing component 1335 is configured to actuate at least one of the two or more extendable and retractable load-bearing feet 1325 based on the information regarding the location of the walking aid 1300 in the environment.

Those having ordinary skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having ordinary skill in the art will recognize that there are various vehicles by which processes and/or systems and/or other technologies disclosed herein can be effected (e.g., hardware, software, and/or firmware), and that the preferred vehicle will vary with the context in which the processes and/or systems and/or other technologies are deployed. For example, if an implementer determines that speed and accuracy are paramount, the surgeon may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies disclosed herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. In a general sense the various aspects disclosed herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices disclosed herein, or a microdigital processing unit configured by a computer program which at least partially carries out processes and/or devices disclosed herein), electrical circuitry forming a memory device (e.g., forms of random access memory), and/or electrical circuitry forming a communications device (e.g., a modem, communications switch, or optical-electrical equipment). The subject matter disclosed herein may be implemented in an analog or digital fashion or some combination thereof.

At least a portion of the devices and/or processes described herein can be integrated into a data processing system. A data processing system generally includes one or more of a system unit housing, memory such as volatile or non-volatile memory, processors such as microprocessors or digital signal processors, computational entities such as operating systems, drivers, and applications programs, one or more interaction devices (e.g., a touch pad, a touch screen, an antenna, etc.), and/or control systems including feedback loops and control motors (e.g., feedback for sensing the walking surface and/or sensing the position of the walking aid relative to the walking surface; control actuators for actuating the extendable and retractable load-bearing feet). A data processing system may be implemented utilizing suitable commercially available components, such as those typically found in data computing/communication and/or network computing/communication systems.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

One skilled in the art will recognize that the herein described component, devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components, devices, and objects should not be taken as limiting.

With respect to the use of substantially any plural and/or singular terms herein, the plural can be translated to the singular and/or from the singular to the plural as is appropriate to the context and/or application. The various singular/plural permutations are not expressly set forth herein for sake of clarity.

In some instances, one or more components can be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) can generally encompass active-state components and/or inactive-state components and/or standby-state components, unless context requires otherwise.

While particular aspects of the present subject matter described herein have been shown and described, changes and modifications can be made without departing from the subject matter described herein and its broader aspects and, therefore, the appended claims are to encompass within their scope all such changes and modifications as are within the true spirit and scope of the subject matter described herein. Terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). If a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). Typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A walking aid comprising:
a linear rod having a first end and a second end;
a hand grip disposed in proximity to the first end of the linear rod;
two or more extendable and retractable load-bearing feet at the second end of the linear rod, at least one of the two or more extendable and retractable load-bearing feet including two or more telescoping segments;
at least one first sensor including circuitry configured to detect a parameter of a walking surface in proximity to the second end of the linear rod and to transform the detected parameter of the walking surface into a first sensor output;
a controller including a microprocessor and circuitry, the controller operably coupled to the two or more extendable and retractable load-bearing feet and the at least one first sensor and including
input circuitry configured to receive the first sensor output including information regarding the detected parameter of the walking surface; and
actuation circuitry configured to actuate at least one of the two or more extendable and retractable load-bearing feet in response to the received information regarding the detected parameter of the walking surface;
a user interface operably coupled to the controller;
at least one second sensor including circuitry configured to detect a parameter of the walking aid and to transform the detected parameter of the walking aid into a second sensor output; and
a transmission unit operably coupled to the at least one second sensor, the transmission unit including an antenna and circuitry configured to transmit the second sensor output to a computing device.

2.-6. (canceled)

7. The walking aid of claim 1, wherein the at least one first sensor comprises at least one of a camera, a proximity sensor, a pressure sensor, an accelerometer, a gyroscope, a tilt sensor, an inclinometer, a tactile sensor, or a texture sensor.

8.-15. (canceled)

16. The walking aid of claim 1, wherein the at least one second sensor comprises at least one of an accelerometer or a gyroscope.

17. (canceled)

18. The walking aid of claim 1, wherein the at least one second sensor comprises at least one of a camera, a GPS receiver, or a clock.

19.-20. (canceled)

21. The walking aid of claim 1, wherein the parameter of the walking aid comprises at least one of movement, speed, location, tip motion of the walking aid, position of the walking aid relative to the walking surface, or tilt angle of the walking aid relative to the walking surface.

22. The walking aid of claim 1, wherein the transmission unit comprises a radiofrequency transmission unit.

23. The walking aid of claim 1, wherein the transmission unit includes circuitry configured to transmit the second sensor output to a mobile communication device.

24. The walking aid of claim 1, further comprising at least one receiver including circuitry configured to receive a signal from the computing device.

25. The walking aid of claim 1, further comprising at least one receiver including circuitry configured to receive a signal from a remote signaling device and to transform the received signal from the remote signaling device into information regarding a location of the walking aid in an environment.

26. The walking aid of claim 1, further comprising at least one receiver including circuitry configured to receive a signal from a wearable device.

27.-29. (canceled)

30. The walking aid of claim 1, further comprising a warning system operably coupled to the controller and configured to alert a user, wherein the warning system comprises at least one of an audible warning system, an optical warning system, or a haptic warning system.

31. The walking aid of claim 1, further comprising at least one third sensor operably coupled to the transmission unit and including circuitry configured to detect a physiological parameter of a user and to transform the detected physiological parameter of the user into a third sensor output, wherein the transmission unit includes circuitry configured to transmit the third sensor output to the computing device, wherein the at least one third sensor includes at least one camera, audio sensor, pressure sensor, load sensor, pulse oximeter, electrocardiogram sensor, temperature sensor, or heart rate monitor.

32.-33. (canceled)

34. A system, comprising:
a walking aid including
a linear rod having a first end and a second end;
a hand grip disposed in proximity to the first end of the linear rod;
two or more extendable and retractable load-bearing feet at the second end of the linear rod, at least one of the two or more extendable and retractable load-bearing feet including two or more telescoping segments;
at least one first sensor including circuitry configured to detect a parameter of a walking surface in proximity to the second end of the linear rod and to transform the detected parameter of the walking surface into a first sensor output;
a controller including a microprocessor and circuitry, the controller operably coupled to the two or more extendable and retractable load-bearing feet and the at least one first sensor and including
input circuitry configured to receive the first sensor output including information regarding the detected parameter of the walking surface, and
actuation circuitry configured to actuate at least one of the two or more extendable and retractable load-bearing feet in response to the received information regarding the detected parameter of the walking surface;
at least one second sensor including circuitry configured to detect a parameter of the walking aid and to transform the detected parameter of the walking aid into a second sensor output; and
a transmission unit operably coupled to the at least one second sensor, the transmission unit including an antenna and circuitry configured to transmit the second sensor output to a computing device; and
the computing device including a processor and circuitry including
input circuitry configured to receive the second sensor output including information regarding the detected parameter of the walking aid;
calculation circuitry configured to calculate a gait value based on the received information regarding the detected parameter of the walking aid; and
reporting circuitry configured to report the calculated gait value.

35. The system of claim 34, wherein the walking aid includes a user-activated control mechanism including circuitry configured to control extension and retraction of the two or more extendable and retractable load-bearing feet.

36.-38. (canceled)

39. The system of claim 34, wherein the at least one first sensor of the walking aid comprises at least one of a camera, a proximity sensor, a pressure sensor, an accelerometer, a gyroscope, a tactile sensor, a tilt sensor, an inclinometer, or a texture sensor.

40. The system of claim 34, wherein the at least one second sensor of the walking aid comprises at least one of a camera, an accelerometer, a gyroscope, a GPS receiver, or a clock.

41.-42. (canceled)

43. The system of claim 34, wherein the walking aid further includes at least one receiver including circuitry configured to receive a signal from the computing device.

44. The system of claim 34, further comprising at least one remote signaling device in wireless communication with at least one receiver associated with the walking aid, the at least one receiver including circuitry configured to receive a signal from the at least one remote signaling device and to transform the received signal into information regarding a location of the walking aid in an environment, wherein the at least one remote signaling device includes one or more beacons configured to emit a radio signal, a magnetic signal, an acoustic signal, an electromagnetic signal, or an optical signal.

45. (canceled)

46. The system of claim 44, wherein the at least one remote signaling device includes a local positioning system.

47. The system of claim 34, wherein the computing device comprises a dedicated handheld device.

48. The system of claim 34, wherein the computing device comprises a mobile communication device.

49. The system of claim 34, wherein the computing device comprises a tablet computer, a laptop computer, or a desktop computer.

50. The system of claim 34, wherein the reporting circuitry is configured to report the calculated gait value to at least one of a digital display, a reporting device, a second computing device, or a network.

51. (canceled)

52. The system of claim 34, wherein the computing device further includes evaluation circuitry configured to evaluate a condition of a user based on a comparison of the calculated gait value with at least one reference gait value; and reporting circuitry configured to report the evaluated condition of the user.

53. The system of claim 52, wherein the reporting circuitry of the computing device is configured to report the evaluated condition of the user to at least one of a haptic reporting device, an audio reporting device, an optical reporting device, or a digital display associated with the walking aid.

54. (canceled)

55. The system of claim 52, wherein the reporting circuitry of the computing device is configured to report the evaluated condition of the user to at least one of a haptic reporting device, an audio reporting device, an optical reporting device, or a digital display associated with the computing device.

56. The system of claim 52, wherein the reporting circuitry of the computing device is configured to report the evaluated condition of the user to at least one of a second computing device or a network.

57.-59. (canceled)

60. The system of claim 34, wherein the walking aid includes at least one third sensor operably coupled to the transmission unit and including circuitry configured to detect a physiological parameter of a user and to transform the detected physiological parameter of the user into a third sensor output, wherein the transmission unit includes circuitry configured to transmit the third sensor output to the computing device, wherein the computing device includes input circuitry configured to receive the third sensor output including the information regarding the detected physiological parameter of the user and reporting circuitry configured to report the detected physiological parameter of the user, wherein the at least one third sensor includes at least one camera, audio sensor, pressure sensor, load sensor, pulse oximeter, electrocardiogram sensor, temperature sensor, or heart rate monitor.

61. (canceled)

62. The system of claim 34, further comprising non-transitory machine readable media including one or more instructions implemented on the computing device, the one or more instructions including
one or more instructions for receiving the second sensor output including information regarding the detected parameter of the walking aid;
one or more instructions for calculating the gait value based on the received information regarding the detected parameter of the walking aid;
one or more instructions for comparing the calculated gait value with at least one reference gait value;
one or more instructions for evaluating a condition of a user based on the comparison between the calculated gait value and the at least one reference gait value; and
one or more instructions for reporting the evaluated condition of the user.

63. A walking aid comprising:
a linear rod having a first end and a second end;
a hand grip disposed in proximity to the first end of the linear rod;
two or more extendable and retractable load-bearing feet at the second end of the linear rod, at least one of the two or more extendable and retractable load-bearing feet including two or more telescoping segments;
at least one first sensor including circuitry configured to detect a parameter of a walking surface in proximity to the second end of the linear rod and to transform the detected parameter of the walking surface into a first sensor output;
at least one second sensor including circuitry configured to detect a parameter of the walking aid and to transform the detected parameter of the walking aid into a second sensor output;
a reporting device; and
a computing component including a microprocessor and circuitry, the computing component operably coupled to the two or more extendable and retractable load-bearing feet, the at least one first sensor, the at least one second sensor, and the reporting device, and including
input circuitry configured to receive the first sensor output including information regarding the detected parameter of the walking surface and to receive the second sensor output including information regarding the detected parameter of the walking aide;
actuation circuitry configured to actuate at least one of the two or more extendable and retractable load-bearing feet in response to the received information regarding the detected parameter of the walking surface;
calculation circuitry configured to calculate a gait value for a user based on the received information regarding the detected parameter of the walking aide; and
reporting circuitry configured to transmit a signal to the reporting device in response to the calculated gait value.

64. The walking aid of claim 63, further comprising a user-activated control mechanism to control extension and retraction of the two or more extendable and retractable load-bearing feet.

65.-68. (canceled)

69. The walking aid of claim 63, wherein at least one of the two or more extendable and retractable load-bearing feet is configured to at least one of retract for storage into an interior portion of the linear rod or retract for storage on a side of the linear rod.

70. The walking aid of claim 63, wherein the at least one first sensor comprises at least one of a camera, a proximity sensor, a pressure sensor, an accelerometer, a gyroscope, a tilt sensor, an inclinometer, a tactile sensor, or a texture sensor.

71.-72. (canceled)

73. The walking aid of claim 63, wherein the parameter of the walking surface includes at least one of a surface type, a friction coefficient, a roughness, a slope, an edge, a height, a distance, or a bump.

74.-76. (canceled)

77. The walking aid of claim 63, wherein the at least one second sensor includes at least one of an accelerometer, a gyroscope, a camera, a GPS receiver, or a clock.

78. The walking aid of claim 63, wherein the parameter of the walking aid comprises at least one of movement, speed, location, tip motion of the walking aid, position of the walking aid relative to the walking surface, or tilt angle of the walking aid relative to the walking surface.

79. The walking aid of claim 63, wherein the reporting device is incorporated into or onto the handgrip.

80. The walking aid of claim 63, wherein the reporting device comprises at least one of an optical reporting device, an audio reporting device, a haptic reporting device, or a digital display.

81. (canceled)

82. The walking aid of claim 63, wherein the reporting device comprises a transmission unit including an antenna.

83.-84. (canceled)

85. The walking aid of claim 82, wherein the transmission unit includes circuitry configured to transmit a signal to a remote computing device, the signal including information regarding the calculated gait value.

86. The walking aid of claim 82, wherein the transmission unit includes circuitry configured to transmit a signal to a mobile communication device, the signal including information regarding the calculated gait value.

87. The walking aid of claim 63, wherein the computing component includes evaluation circuitry configured to evaluate a condition of the user based on a comparison of the calculated gait value with at least one reference gait value; and the reporting circuitry is configured to transmit a signal to the reporting device in response to the evaluated condition of the user.

88.-89. (canceled)

90. The walking aid of claim 63, further comprising at least one third sensor operably coupled to the computing component and including circuitry configured to detect a physiological parameter of a user and to transform the detected physiological parameter of the user into a third sensor output, wherein the input circuitry is configured to receive the third sensor output including information regarding the detected physiological parameter of the user, and the reporting circuitry is configured to transmit a signal to the reporting device in response to the received information regarding the detected physiological parameter of the user, wherein the at least one third sensor includes at least one camera, audio sensor, pressure sensor, load sensor, pulse oximeter, electrocardiogram sensor, temperature sensor, or heart rate monitor.

91.-92. (canceled)
